(19) United States
(12) Patent Application Publication
(10) Pub. No.: US 2012/0220594 A1
(43) Pub. Date: Aug. 30, 2012

(54) METHODS FOR TREATING CANCER IN PATIENTS HAVING IGF-1R INHIBITOR RESISTANCE

(75) Inventors: Fei Huang, Princeton, NJ (US); Joan Carboni, Yardley, PA (US)

(73) Assignee: Bristol-Meyers Squibb Company

(21) Appl. No.: 13/504,498

(22) PCT Filed: Oct. 29, 2010
(86) PCT No.: PCT/US10/54704

§ 371 Date: Apr. 27, 2012

Related U.S. Application Data
(60) Provisional application No. 61/280,275, filed on Oct. 30, 2009.
(60) Provisional application No. 61/300,082, filed on Feb. 1, 2010.

(51) Int. Cl. A61K 31/53; A61P 35/00; C12Q 1/68

(52) U.S. Cl. 514/243; 435/6.13; 435/6.11

(57) ABSTRACT

A method for treating cancer comprising identifying a mammal that overexpresses breast cancer resistance protein; and administering to said mammal a pharmaceutical composition comprising a therapeutically effective amount of ixabepilone. In one aspect, the mammal is not administered an agent that is susceptible to breast cancer resistance protein overexpression resistance. In another aspect, the cancer is a solid tumor.

This application claims benefit to provisional application U.S. Ser. No. 61/280,275 filed Oct. 30, 2009; and to provisional application U.S. Ser. No. 61/300,082, filed Feb. 1, 2010; under 35 U.S.C. §119(e). The entire teachings of the referenced applications are incorporated herein by reference.

### FIELD OF THE INVENTION

The present invention relates generally to the field of pharmacogenomics, and more specifically to methods and procedures to determine drug sensitivity in patients to allow the identification of individualized genetic profiles which will aid in treating diseases and disorders.

### BACKGROUND OF THE INVENTION

Targeted agents have emerged as important therapies in the treatment of a variety of human malignancies. Initial success is often hampered by a relatively rapid acquisition of drug resistance and subsequent relapse particularly in patients with advanced disease. Like conventional chemotherapy drugs, to which resistance has been well established as an important challenge in cancer therapy, the more recently developed kinase inhibitors are also subject to acquired resistance (Janne et al., Nat. Rev. Drug Discov., 8(9):709-723 (2009); Engelman et al., Curr. Opin. Genet. Dev., 18:73-79 (2008)). The mechanisms of acquired drug resistance are beginning to be elucidated largely through two strategies: one is the molecular analysis of clinical specimens from patients who initially had clinical response to treatment therapy then relapsed on the drug; another is through in vitro cell culture modeling. The latter involves culturing drug-sensitive tumor-derived cell lines in the presence of continuous drug exposure until most of the cells are eliminated and then the cultures are eventually enriched with drug-resistant cell populations, which then can be characterized by genomic approaches to identify resistance mechanisms (Janne et al., Nat. Rev. Drug Discov., 8(9):709-723 (2009); Engelman et al., Curr. Opin. Genet. Dev., 18:73-79 (2008)).

For example, utilizing both approaches has revealed several mechanisms for acquired resistance to imatinib, a BCR-ABL inhibitor. The molecular basis for acquired resistance to imatinib involves kinase domain mutations (Gorre et al., Science, 293(5531):876-880 (2001)), BCR-ABL amplification or overexpression (Gorre et al., Science, 293(5531):876-880 (2001)), activation of BCR-ABL independent pathways, such as members of Src family kinases (Donato et al., Blood, 101(2):690-698 (2003)) and AXL (Mahadevan et al., Oncogene, 26:3909-3919 (2007)), and P-glycoprotein efflux pump overexpression (Illmer et al., Leukemia, 18(3):401-408 (2004)). Understanding the mechanisms for acquired resistance to imatinib provided the basis for the development of second-generation BCR-ABL inhibitors such as dasatinib and nilotinib.

Since activation and expression of insulin-like growth factor (IGF) signaling components contribute to proliferation, survival, angiogenesis, metastasis, and resistance to anti-cancer therapies in many human malignancies (7), the IGF system has become an attractive therapeutic target. The IGF system consists of two closely related receptors insulin receptor (IR), the type I-IGF receptor (IGF-1R), and three ligands (IGF-I, IGF-II, and insulin). IR/IGF1R hybrid receptors signal similarly to IGF1R holoreceptors and have recently been implicated in cancer (Denley et al., Cytokine Growth Factor Rev., 16:421-439 (2005); Pandini et al., Clin. Cancer Res., 5:1935-1944 (1999)).

Insulin receptor plays an important role in regulating IGF action, either as a hybrid or holoreceptor, and IGF-1R/IR hybrid receptors are activated by IGF-I and IGF-II (Morrione et al., Proc. Natl. Acad. Sci. USA, 94:3777-3782 (1997)) Inhibition of both IGF-1R and IR may be necessary to completely disrupt the malignant phenotype regulated by this signaling pathway (Law et al., Cancer Res., 68(24):10238-10246 (Dec. 15, 2008)). IGF-IR is becoming one of the most intensively investigated molecular targets in oncology. Currently, there are close to 30 drug candidates being investigated that target the IGF-IR/IR receptors and a number of them are in clinical trials including IGF-1R antibodies and small molecule inhibitors (Gualberto et al., Oncogene, 28(34):3009-3021 (2009); Rodon et al., Mol. Cancer Ther., 7(9):2575-2588 (2008); Weroha et al., J. Mamm. Gland Biol. Neoplasia, 13:471-483 (2008)).

BMS-754807 is a potent and selective reversible small molecule inhibitor of IGF1R family kinases, it targets both IGF-1R and IR and has a wider spectrum of antitumor efficacy (Carboni et al., “BMS-754807, a small molecule inhibitor of IGF1R for clinical development”, Proceedings of the 100th Annual Meeting of the American Association for Cancer Research, 2009 Apr. 18-22, Denver, Colo., Abstract No. 1742). Targeting IGF-1R/IR signaling results in cancer cell growth inhibition both in vitro and in vivo by BMS-754807. This drug is currently in phase I development for the treatment of a variety of human cancers and pre-clinical defined efficacious exposures have been achieved with oral administration of single, tolerable doses in humans (Clements et al., AACR-NCI-EORTC Molecular Targets and Cancer Therapeutics Meeting 2009, Abstract No. A101) and pharmacological activity of BMS-754807 on pharmacodynamic biomarkers has been observed in cancer patients (Desai et al., AACR-NCI-EORTC Molecular Targets and Cancer Therapeutics Meeting 2009, Abstract No. A109).

Early clinical evidence has demonstrated that anti-IGF-1R antibodies have promising clinical benefit as a single agent or in combination with chemotherapy (Olmos et al., J. Clin. Oncol., 26:553s (2008); Tolcher et al., J. Clin. Oncol., 25:118s (2007); Karp et al., ASCO Meeting Abstracts 2008, 26 15_suppl:8015; Haluska et al. ASCO Meeting Abstracts 2007, 25 18_suppl:3586). With increasing numbers of small molecular IGF-1R inhibitors entering clinical testing, it is highly probable they will soon provide definitive data on their value in future cancer treatments. However, like other cancer drugs, the IGF-1R antibodies and small molecule inhibitors could also face a very important and general drawback, i.e., development of resistance.

New prognostic and predictive markers, which may facilitate individualized patient therapy are needed to accurately predict patient response to treatments, and in particular, identify the development of resistance to small molecule or biological molecule drugs, in order to identify the best treatment regimens. The problem may be solved by the identification of new parameters that could better predict the patient's sensitivity to treatment. The classification of patient samples is a crucial aspect of cancer diagnosis and treatment. The association of a patient's response to a treatment with molecular and genetic markers can open up new opportunities for treatment development in non-responding patients, or distinguish a treatment's indication among other treatment choices because of higher confidence in the efficacy. Further, the pre-selection of patients who are likely to respond well to a medicine, drug, or combination therapy may reduce the number of patients needed in a clinical study or accelerate the time needed to complete a clinical development program (Cockett, M. et al., Curr. Opin. Biotechnol., 11:602-609 (2000)).

The ability to determine which patients are responding to IGF-1R/IR therapies or predict drug sensitivity in patients is particularly challenging because drug responses reflect not only properties intrinsic to the target cells, but also a host's metabolic properties. Efforts to use genetic information to predict or monitor drug response have primarily focused on individual genes that have broad effects, such as the multidrug resistance genes mdr1 and mrp1 (Sonneveld, P., J. Intern. Med., 247:521-534 (2000)).

The development of microarray technologies for large scale characterization of gene mRNA expression pattern has made it possible to systematically search for molecular markers and to categorize cancers into distinct subgroups not evident by traditional histopathological methods (Khan, J. et al., Cancer Res., 58:5009-5013 (1998); Alizadeh, A. A. et al., Nature, 403:503-511 (2000); Bittner, M. et al., Nature, 406:536-540 (2000); Khan, J. et al., Nature Medicine, 7(6):673-679 (2001); and Golub, T. R. et al., Science, 286:531-537 (1999); Alon, U. et al., Proc. Natl. Acad. Sci. USA, 96:6745-6750 (1999)). Such technologies and molecular tools have made it possible to monitor the expression level of large numbers of transcripts within a cell population at any given time (see, e.g., Schena et al., Science, 270:467-470 (1995); Lockhart et al., Nature Biotechnology, 14:1675-1680 (1996); Blanchard et al., Nature Biotechnology, 14:1649 (1996); U.S. Pat. No. 5,569,588 to Ashby et al.).

Recent studies demonstrate that gene expression information generated by microarray analysis of human tumors can predict clinical outcome (van't Veer, L. J. et al., Nature, 415:530-536 (2002); Shipp, M. et al., Nature Medicine, 8(1):68-74 (2002); Glinsky, G. et al., J. Clin. Invest., 113(6):913-923 (2004)). These findings bring hope that cancer treatment will be vastly improved by better predicting and monitoring the response of individual tumors to therapy.

Needed are new and alternative methods and procedures to determine drug sensitivity or monitor response in patients to allow the development of individualized diagnostics which may be beneficial to treating diseases and disorders based on patient response at the molecular level, particularly cancer.

### SUMMARY OF THE INVENTION

The invention provides methods and procedures for determining patient sensitivity to one or more IGF-1R agents.

The present invention relates to the identification of several biomarkers for use in identifying resistance to IGF-1R inhibition. Specifically, the invention is directed to methods of identifying patients who may be susceptible to IGF-1R inhibitor resistance, or who are resistant to IGF-1R inhibition, comprising the step of measuring the expression level of PDGFR-α in a patient, wherein an elevated level of PDGFR-α relative to a control is indicative of resistance to IGF-1R inhibition.

The present invention relates to the identification of several biomarkers for use in identifying resistance to IGF-1R inhibition. Specifically, the invention is directed to methods of identifying patients who may be susceptible to IGF-1R inhibitor resistance, or who are resistant to IGF-1R inhibition, comprising the step of measuring the expression level of c-KIT in a patient, wherein an elevated level of c-KIT relative to a control is indicative of resistance to IGF-1R inhibition.

The invention is also directed to methods of identifying patients who may be susceptible to IGF-1R inhibitor resistance, or who are resistant to IGF-1R inhibition, comprising the step of measuring the expression level of AXL in a patient, wherein an elevated level of AXL relative to a control is indicative of resistance to IGF-1R inhibition.

The invention is also directed to methods of identifying patients who may be susceptible to developing resistance to treatment with a small molecule IGF-1R inhibitor, or who are resistant to small molecule IGF-1R inhibition, comprising the step of measuring the expression level of AXL in a patient, wherein a diminished level of AXL relative to a control is indicative of resistance to small molecule IGF1R inhibition. For the purposes of the present invention, small molecule IGF-1R inhibitors include small molecules, adnectins, siRNAs, iRNA, and antisense molecules.

The invention is also directed to methods of identifying patients who may be susceptible to developing resistance to treatment with a monoclonal antibody-based IGF-1R inhibitor, or who are resistant to a monoclonal antibody-based IGF-1R inhibitor, comprising the step of measuring the expression level of AXL in a patient, wherein an elevated level of AXL relative to a control is indicative of resistance to monoclonal antibody-based IGF1R inhibition. For the purposes of the present invention, monoclonal antibody-based IGF-1R inhibitors include adnectins, single chain antibodies, domain antibodies, antibody fragments, etc.

The present invention also relates to methods for identifying patients who are resistant to, or have developed resistance to, or have a high likelihood of developing resistance to, inhibition by an IGF-1R antibody and that may benefit from the administration of an IGF-1R small molecule inhibitor, comprising the step of: (i) screening a biological sample, for cells that are resistant, or partially resistant, or do not respond, or that have stopped responding, or that have a diminished response, to one or more IGF-1R antibody inhibitors; and (ii) screening cells from said patient for increased expression of one or more of the following markers: IGFBP3, IGFBP5, IGFBP6, AXL, c-KIT, and PDGFR-α, relative to a standard, wherein if overexpression of one or more of said markers is present, administering a therapeutically acceptable amount of a small molecule IGF-1R inhibitor, a more aggressive dosing regimen of a small molecule IGF-1R inhibitor, an increased dose of a small molecule IGF-1R inhibitor, or administering an IGF-1R inhibitor in combination with one or more IGF-1R inhibitors and/or other agents, such as for example, an EGFR inhibitor and/or a PDGFR-α inhibitor. Wherein said the cancer is a solid tumor, an advanced solid tumor, a metastatic solid tumor, a neoplasm, sarcoma, colon, and/or breast cancer, or other cancer outlined herein. In yet another aspect, said method comprises the additional step of determining whether said patient has a diminished expression level of IGF-1R. In one aspect, the mammal is a human.

The present invention also relates to methods for identifying patients who are resistant to, or have developed resistance to, or have a high likelihood of developing resistance to, inhibition by an IGF-1R antibody and that may benefit from the administration of an IGF-1R small molecule inhibitor, comprising the step of: (i) screening a biological sample, for cells that are resistant, or partially resistant, or do not respond, or that have stopped responding, or that have a diminished response, to one or more IGF-1R antibody inhibitors; and (ii) screening cells from said patient for increased expression of one or more of the following markers: AXL; relative to a standard, wherein if decreased expression of one or more of said markers is present, administering a therapeutically acceptable amount of a small molecule IGF-1R inhibitor, a more aggressive dosing regimen of a small molecule IGF-1R inhibitor, an increased dose of a small molecule IGF-1R inhibitor, or administering an IGF-1R inhibitor in combination with one or more IGF-1R inhibitors and/or other agents, such as for example, an EGFR inhibitor and/or a PDGFR-α inhibitor. Wherein said the cancer is a solid tumor, an advanced solid tumor, a metastatic solid tumor, a neoplasm, sarcoma, colon, and/or breast cancer, or other cancer outlined herein. In yet another aspect, said method comprises the additional step of determining whether said patient has a diminished expression level of IGF-1R. In one aspect, the mammal is a human.

The invention is also directed to methods of identifying patients who may be susceptible to IGF-1R inhibitor resistance, or who are resistant to IGF-1R inhibition, comprising the step of measuring the expression level of IGF-1R in a patient, wherein a decreased level of IGF1R relative to a control is indicative of resistance to IGF-1R inhibition.

The invention is also directed to methods of identifying patients who may be susceptible to IGF-1R inhibitor resistance, or who are resistant to IGF-1R inhibition, comprising the step of measuring the expression level of IGFBPs, such as IGFBP3, IGFBP5, and/or IGFBP6, in a patient, wherein an elevated level of IGFBPs relative to a control is indicative of resistance to IGF-1R inhibition.

The present invention also relates to methods of identifying patients who may be susceptible to IGF-1R inhibitor resistance, or who are resistant to IGF-1R inhibition, comprising the step of treating IGF-1R inhibitor-resistant patients with the synergistic combination of an IGF-1R inhibitor with a PDGFR-α inhibitor.

The present invention also relates to methods of identifying patients who may be susceptible to IGF-1R inhibitor resistance, or who are resistant to IGF-1R inhibition, comprising the step of identifying patients who may benefit from the combination of an IGF-1R inhibitor and a PDGFR-α inhibitor comprising the step of determining whether the level of IGFR1 is elevated relative to a control, wherein a decreased level of IGFR1 suggests a patient may benefit from the administration of said combination.

The present invention also relates to methods of identifying patients who may benefit from the combination of an IGF-1R inhibitor with an EGFR inhibitor comprising the step of determining a condition selected from the group consisting of: (a) whether the level of IGF-1R is decreased relative to a control; (b) whether the level of one or more of IGFBP3, IGFBP5, or IGFBP 6 is decreased relative to a control; and (c) whether the level of EGFR is elevated relative to a control; wherein a decreased IGF-1R level, an elevated level of one or more of IGFBP3, IGFBP5, or IGFBP 6 suggests a patient will benefit from the administration of said combination.

In one aspect, the invention relates to a method for treating cancer comprising identifying a mammal that has a diminished expression level of IGF-1R; and administering to said mammal a pharmaceutical composition comprising a therapeutically effective amount of an IGF-1R inhibitor, either alone or in combination with an EGFR inhibitor and/or a PDGFR-α inhibitor. In another aspect, the cancer is a solid tumor, an advanced solid tumor, a metastatic solid tumor, a neoplasm, sarcoma, colon, and/or breast cancer, or other cancer outlined herein. In yet another aspect, the mammal overexpresses one or more of the following: IGFBP3, IGFBP5, IGFBP 6, and PDGFR-α. In one aspect, the mammal is a human.

In one aspect, the invention relates to a method for treating cancer comprising identifying a mammal that has a diminished expression level of IGF-1R; and administering to said mammal a pharmaceutical composition comprising a therapeutically effective amount of an IGF-1R inhibitor, either alone or in combination with an EGFR inhibitor and/or a PDGFR-α inhibitor. In another aspect, the cancer is a solid tumor, an advanced solid tumor, a metastatic solid tumor, a neoplasm, sarcoma, colon, and/or breast cancer, or other cancer outlined herein. In yet another aspect, the mammal has decreased expression of one or more of the following: AXL. In one aspect, the mammal is a human.

The present invention provides a method of screening a biological sample, for cells that are resistant, or partially resistant, or do not respond, or that have stopped responding, or that have a diminished response, to one or more IGF-1R inhibitors. For example, the present invention provides a method of screening cells from an individual suffering from cancer who is either being treated with one or more IGF-1R inhibitors or is naïve to said agents, and whose cells do not respond or have stopped responding or that have a diminished response to one or more IGF-1R inhibitors, for decreased expression of IGF-1R relative to a standard. If decreased expression of IGF-1R is present, administration of a therapeutically acceptable amount of an IGF-1R inhibitor, alone or in combination with one or more IGF-1R inhibitors and/or other agent, such as an EGFR inhibitor and/or a PDGFR-α inhibitor, may be suggested to inhibit proliferation of said cells. Wherein said the cancer is a solid tumor, an advanced solid tumor, a metastatic solid tumor, a neoplasm, sarcoma, colon, and/or breast cancer, or other cancer outlined herein. In yet another aspect, the mammal overexpresses one or more of the following: IGFBP3, IGFBP5, IGFBP 6, and PDGFR-α. In another aspect, the mammal has decreased expression level of AXL. In one aspect, the mammal is a human.

The present invention provides a method of identifying a treatment regimen for a patient suffering from cancer comprising the step of: (i) screening a biological sample, for cells that are resistant, or partially resistant, or do not respond, or that have stopped responding, or that have a diminished response, to one or more IGF-1R inhibitors; and (ii) screening cells from said patient for decreased expression of IGF-1R relative to a standard, wherein if decreased expression of IGF-1R is present, administering a therapeutically acceptable amount of an IGF-1R inhibitor, a more aggressive dosing regimen of an IGF-1R inhibitor, an increased dose of an IGF-1R inhibitor, or administering an IGF-1R inhibitor in combination with one or more IGF-1R inhibitors and/or other agents, such as for example, an EGFR inhibitor and/or a PDGFR-α inhibitor. Wherein said the cancer is a solid tumor, an advanced solid tumor, a metastatic solid tumor, a neoplasm, sarcoma, colon, and/or breast cancer, or other cancer outlined herein. In yet another aspect, said method comprises the additional step of determining whether said patient overexpresses one or more of the following: IGFBP3, IGFBP5, IGFBP6, AXL, c-KIT, and PDGFR-α. In another aspect, the mammal has decreased expression level of AXL. In one aspect, the mammal is a human.

The present invention provides a method of identifying a treatment regimen for a patient suffering from cancer comprising the step of: (i) screening a biological sample, for cells that are resistant, or partially resistant, or do not respond, or that have stopped responding, or that have a diminished response, to one or more IGF-1R inhibitors; and (ii) screening cells from said patient for increased expression of one or more of the following markers: IGFBP3, IGFBP5, IGFBP6, AXL, c-KIT, and PDGFR-α, relative to a standard, wherein if overexpression of one or more of said markers is present, or if decreased expression of AXL is present, administering a therapeutically acceptable amount of an IGF-1R inhibitor, a more aggressive dosing regimen of an IGF-1R inhibitor, an increased dose of an IGF-1R inhibitor, or administering an IGF-1R inhibitor in combination with one or more IGF-1R inhibitors and/or other agents, such as for example, an EGFR inhibitor and/or a PDGFR-α inhibitor, and/or paxlitaxol. Wherein said the cancer is a solid tumor, an advanced solid tumor, a metastatic solid tumor, a neoplasm, sarcoma, colon, and/or breast cancer, or other cancer outlined herein. In yet another aspect, said method comprises the additional step of determining whether said patient has a diminished expression level of IGF-1R. In one aspect, the mammal is a human.

In another embodiment of the present invention, a combination of the present invention may also encompass the combination of an IGF-1R inhibitor with paxlitaxel and/or carboplatin and/or HERCEPTIN®.

The diagnostic methods of the invention can be, for example, an in vitro method wherein the step of measuring in the mammal the level of at least one biomarker comprises taking a biological sample from the mammal and then measuring the level of the biomarker(s) in the biological sample. The biological sample can comprise, for example, at least one of serum, whole fresh blood, peripheral blood mononuclear cells, frozen whole blood, fresh plasma, frozen plasma, urine, saliva, skin, hair follicle, bone marrow, or tumor tissue.

The level of the at least one biomarker can be, for example, at the level of protein and/or mRNA transcript of the biomarker(s).

The invention also provides an isolated IGF-1R biomarker, an isolated IGFBP3 biomarker, an isolated IGFBP5 biomarker, an isolated IGFBP6 biomarker, an isolated AXL biomarker, and PDGFR-α biomarkers. The biomarkers of the invention include nucleotide and/or amino acid sequences of sequences that are at least 90%, 95%, 96%, 97%, 98%, 99%, and 100% identical to the sequences provided as gi|NP_{—}000866 (SEQ ID NO:1 and 2), gi|NM_{—}000875 (IGF-1R) (SEQ ID NO:3 and 4); gi|NP_{—}006197 (SEQ ID NO:5 and 6), gi|NM_{—}006206 (PDGFR-α) (SEQ ID NO:7 and 8); gi|NP_{—}001690 (SEQ ID NO:9 and 10), gi|NP_{—}068713 (SEQ ID NO:11 and 12), gi|NM_{—}001699 (SEQ ID NO:13 and 14), gi|NM_{—}021913 (AXL) (SEQ ID NO:15 and 16); gi|NP_{—}000589 (SEQ ID NO:17 and 18), gi|NP_{—}001013416 (SEQ ID NO:19 and 20), gi|NM_{—}000598 (SEQ ID NO:21 and 22), gi|NM_{—}001013398 (SEQ ID NO:23 and 24) (IGFBP3); gi|NP_{—}001543 (SEQ ID NO:25 and 26), gi|NM_{—}001552 (SEQ ID NO:27 and 28) (IGFBP4); gi|NP_{—}000590 (SEQ ID NO:29 and 30), gi|NM_{—}000599 (SEQ ID NO:31 and 32) (IGFBP5); gi|NP_{—}002169 (SEQ ID NO:33 and 34), gi|NM_{—}002178 (SEQ ID NO:35 and 36) (IGFBP6); full-length IGF-1R, IGFBP3, as well as fragments and variants thereof.

The invention also provides a biomarker set comprising two or more biomarkers of the invention.

The invention also provides kits for measuring diminished expression of IGF-1R and/or overexpression of one or more of the following: IGF-1R, IGFBP3, IGFBP5, IGFBP 6, AXL, c-KIT, and/or PDGFR-α, and/or decreased expression of AXL, biomarkers and uses thereof. The invention also provides antibodies, including polyclonal or monoclonal, directed to IGF-1R, IGFBP3, IGFBP5, IGFBP 6, c-KIT, AXL, and/or PDGFR-α protein, and uses thereof in detecting expression levels of said biomarkers.

The present invention provides a method for predicting the likelihood a patient will respond therapeutically to a cancer treatment comprising the administration of an IGF-1R inhibitor, wherein said prediction method comprises the steps of: (a) measuring the level of IGF-1R in a sample from said patient; (b) comparing the level of IGF-1R in said sample relative to a standard, wherein an increased expression level indicates an increased likelihood said patient will respond therapeutically to said cancer treatment; and optionally comprising the step of administering said IGF-1R inhibitor.

The present invention provides a method for predicting the likelihood a patient will respond therapeutically to a cancer treatment comprising the administration of an IGF-1R inhibitor, wherein said prediction method comprises the steps of: (a) measuring the level of a biomarker in a sample from said patient, wherein said biomarker is selected from the group consisting of: IGFBP3; IGFBP5, IGFBP6; AXL; and PDGFR-α; (b) comparing the level of said biomarker in said sample relative to a standard, wherein an increased expression level indicates a decreased likelihood said patient will respond therapeutically to said cancer treatment; and optionally comprising the step of administering said IGF-1R inhibitor.

The present invention provides a method for treating a patient with cancer comprising the steps of: (a) measuring the level of a IGF-1R in a sample from said patient; (b) comparing the level of IGF-1R in said sample relative to a standard, wherein a decreased expression level indicates a decreased likelihood said patient will respond therapeutically to said cancer treatment; and optionally comprising the step of administering said IGF-1R inhibitor.

The present invention provides a method for treating a patient with cancer comprising the steps of: (a) measuring the level of a biomarker in a sample from said patient, wherein said biomarker is selected from the group consisting of: IGFBP3; IGFBP5, IGFBP6; AXL; and PDGFR-α; (b) comparing the level of said biomarker in said sample relative to a standard, wherein an increased expression level indicates a decreased likelihood said patient will respond therapeutically to a treatment comprising an IGF-1R inhibitor; and optionally comprising the step of administering said IGF-1R inhibitor.

The present invention provides a method of identifying a treatment regimen for a patient, comprising the steps of: (a) measuring the level of a IGF-1R in a sample from said patient; (b) comparing the level of IGF-1R in said sample relative to a standard, wherein a decreased expression level indicates a decreased likelihood said patient will respond therapeutically to a treatment comprising an IGF-1R inhibitor, and recommending a more aggressive therapy; and optionally comprising the step of administering said more aggressive therapy; wherein said more aggressive therapy comprises a member of the group consisting of: (a) administering a higher dose of said IGF-1R inhibitor; (b) administering said IGF-1R inhibitor at an increased frequency; and (c) administering said IGF-1R inhibitor in combination with another therapy.

The present invention provides a method of identifying a treatment regimen for a patient, comprising the steps of: (a) measuring the level of a biomarker in a sample from said patient, wherein said biomarker is selected from the group consisting of: IGFBP3; IGFBP5, IGFBP6; AXL; and PDGFR-α; (b) comparing the level of said biomarker in said sample relative to a standard to permit assignment of said sample to either being a member of an overexpression positive class or an overexpression negative class, wherein an overexpression positive sample member indicates a decreased likelihood said patient will respond therapeutically to a treatment comprising an IGF-1R inhibitor, and recommending a more aggressive therapy; and optionally comprising the step of administering said IGF-1R inhibitor; wherein said more aggressive therapy comprises a member of the group consisting of: (a) administering a higher dose of said cancer treatment; (b) administering said cancer treatment at an increased frequency; and (c) administering said cancer treatment in combination with another therapy.

The present invention provides a method of overcoming or preventing acquired resistance to an antibody IGF-1R inhibitor, comprising administering a combination of a second IGF-1R inhibitor with a PDGFR-α inhibitor; wherein said second IGF-1R inhibitor is BMS-754807.

The present invention provides a kit for use in treating a patient with cancer, comprising: (a) a means for measuring whether a sample from said patient is positive for overexpression of one or more of: IGFBP3; IGFBP5, IGFBP6; AXL; and PDGFR-α; (b) a therapeutically effective amount of an IGF-1R inhibitor in combination with a PDGFR-α inhibitor; and optionally comprising the step of administering said IGF-1R inhibitor.

The present invention provides a method according to any of the embodiments outlined herein wherein said measurement is performed using a method selected from the group consisting of: (a) PCR; (b) RT-PCR; (c) FISH; (d) IHC; (e) immunodetection methods; (f) Western Blot; (g) ELISA; (h) radioimmuno assays; (i) immunoprecipitation; (j) FACS (k) HPLC; (l) surface plasmon resonance; (m) optical spectroscopy; and (i) mass spectrometry.

The present invention provides a method according to any of the embodiments outlined herein, wherein said cancer is a solid tumor, a metastatic tumor, breast cancer or lung cancer.

The invention will be better understood upon a reading of the detailed description of the invention when considered in connection with the accompanying figures.

### BRIEF DESCRIPTION OF THE DRAWINGS

FIGS. 1A-F show decreased IGF-1R expression correlated with acquired resistance to IGF-1R inhibitors. Plate A shows IGF-1R RNA level is significantly down regulated in 807R, but not in MAB391R resistance cells compared to the sensitive parental cells as measured by Affymetrix GENECHIP®. Plate B shows differential expression levels of IGF-1R protein expression as measured by western blot. IGF-1Rβ antibody is from Santa Cruz and β-ACTIN® from Chemicon International. Plate C shows western blot to show the recovery of IGF-1R protein expression in MAB391R cells after MAB391 was washed out. Plate D shows flow cytometry analysis of cell surface IGF-1R protein expression in MAB391R cells in the drug washout experiments. Fluorescence intensity α-axis) is directly correlated with the amount of IGF-1R (red). Plate E shows comparison of signaling in response to IGF-1 stimulation between 807R, MAB391R and the parental sensitive cell lines as measured by western blots.

FIGS. 2A-D show cross comparisons of genes that are commonly or differentially expressed between 807R and MAB391R cell lines. Plate A shows a comparison of 807R with MAB391R. t-test analyses identified the number of genes significantly changed expression level (p<0.05 and fold change>2) when compared the gene expression profiling of 807R or MAB391R with that of sensitive parental cells. Overlapped genes have four different patterns (A, B, C and D) as indicated. Plate B shows gene expression pattern in sensitive parental, 807R,807Rout and MAB391R cells for the overlapped genes as shown in FIG. 2A. Cluster A: Genes with decreased levels in both 807R and MAB391R; Cluster B: Genes with increased levels in MAB391R, but decreased in 807R; Cluster C: Genes with increased levels in 807R, but decreased in MAB391R; Cluster D: Genes with increased levels in both 807R & MAB391R. Data from duplicated samples are shown. Plate C shows a pathway analyses by Ingenuity Pathways Analysis software using the genes that are differentially expressed that are unique in 807R or in MAB391R cells. The selected top biological functions and canonical pathways are compared for both cell lines. Plate D shows SNP-Chip analysis of chromosome 4q11-q21 in Rh41 and Rh41-807R cells. The top panel is the data from Rh41 cells, with mostly normal copy number. The bottom panel is the data from Rh41_{—}807R cells, with amplifications in 4g12-4-q21. Compared to Rh41, amplification of chromosome 4q11-q12 containing PDGFR-α and c-KIT genes is detected in 807R cells.

FIG. 2C-Legend:

### Biological Functions

### A=Cell Death

### B=Cellular Movement

### C=Cell Morphology

### D=Cellular Growth and Proliferation

### E=Cell-To-Cell Signaling and Interaction

### F=Cellular Function and Maintenance

### G=Drug Metabolism

### H=Cell Cycle

### I=DNA Replication, Recombination, and Repair

### J=Cell Signaling

### K=Cellular Response to Therapeutics

### Canonical Pathways

### A=IGF-1 Signaling

### B=PTEN Signaling

### C=PDGF Signaling

D=mTOR Signaling

### E=Growth Hormone Signaling

### F=EGF Signaling

### G=SAPK/JNK Signaling

### H=TGF-13 Signaling

### I=VEGF Signaling

### J=PI3K/AKT Signaling

### K=FGF Signaling

### L=Death Receptor Signaling

FIGS. 3A-F show expression pattern comparisons of selected genes/proteins that uniquely changed expression only in 807R or in MAB391R. Plate A shows PDGFR-α RNA expression is up-regulated in 807R but not in MAB391R compared to the sensitive parental cells as measured by Affymetrix GENECHIP®. Insert: PDGFR-α protein expression pattern as detected by western blot. Plate B shows over-expression and constitutively activation of PDGFR-α protein in 807R cells compare to the sensitive parental cells. Cells starved for 24 hr, then stimulated with or without 50 ng/ml PDGF ligand fro 5 min. Cells were lyzed and 30 ug of total protein was used for western blot analyses. Plate C shows RNA expression of c-KIT, FGFR2 and EPHA3 are up-regulated in 807R but not in MAB391R compared to the sensitive parental cells as measured by Affymetrix GENECHIP®. Plate D shows AXL RNA expression is up-regulated in MAB391R but down regulated in 807R compared to the sensitive parental cells as measured by Affymetrix GENECHIP®. Insert: AXL protein expression as detected by western blot. Plate E shows a comparison of IGF-1R and PDGFR-α protein expression in Rh41-S and -807R cells and tumors. Plate F shows the induction of PDGFRα in sensitive parental Rh41cells following treatment with BMS-754807 and two other small molecule IGF-1R inhibitors is concentration-dependent, with increased expression being an early event and observable at 4 hrs after treatment. Plate G shows PDGFRα transcript induction by BMS-754807 in Rh41-S cells is an early event and dose-dependent. Cells treated with 10 nM and 50 nM of BMS-754807 for 1, 2, 4, 8 and 24 hrs. PDGFRα RNA levels were measured by qRT-PCR and compared to the untreated time 0 control.

FIGS. 4A-D show PDGFR-α confers resistance of 807R cells to BMS-754807. Plate A shows BMS-754807 resistant 807R cells are more sensitive to PDGFR-α inhibitor dovitinib than the BMS-754807 sensitive cells Rh41. Plate B shows PDGFR-α expression knockout by PDGFR-α specific siRNA in 807R cells. PDGFRα expression knockdown by siRNA regains the sensitivity to BMS-754807 in Rh41-807R cells 72 hr-post transfection. The average IC₅₀ values and SD from 3 independent experiments are shown. Plate C shows schematic illustration of acquired resistance mechanisms to BMS-754807. (a) Sarcoma cells with high expression of IGF-1R and activated pathway demonstrate response to IGF-1R inhibitor such as BMS-754807 and result in suppression of downstream survival effectors, including PI3K, AKT and ERK, and consequent cell death. (b) Cells with acquired resistance to IGF-1R inhibitor arise through amplification of the gene encoding PDGFR-α kinase, which transduces the redundant survival signals possibly through increased PDGFR-α activation.

FIGS. 5A-D shows synergistic activity is observed when the IGF-1R inhibitor BMS-754807 was combined with a variety of different PDGFR inhibitors. A dilution of ratios drug combination method was used in cellular proliferation assays to test the IC₅₀ values of single agent (A or B) as well as in combination (a or b). IC₅₀ values are shown with standard error, data representative of n=3. “A” or “a” represents BMS-754807 and “B” or “b” represents individual PDGFR-α inhibitor as indicated. Results are derived from 1:1 ratio of two drugs in combination. Combination Index=(a/A+b/B). Combination Index ±95% confidence interval less than 1 indicates synergy.

FIGS. 6A-D show genes that have a unique expression pattern in the MCF7-807R and MAB391R IGF-1R inhibitor resistant cells. The fold change was relative to the level of the sensitive parental line. Plate A. IGF-2 and IGF2R expression change only seen in Rh41-807R and Rh41-807Rout. Plate B. Genes with expression changed only in MAB391R cell line but no significant change was observed in the 807R cell line. Plate C. Genes up-regulated only in 807R cell line but no significant change in MAB391R cell line. Plate D. Genes down-regulated only in 807R cell line but no significant change in MAB391R cell line.

FIG. 7 shows a copy number analysis of chromosome 4 in q11-q21 region. Both IGF-1R inhibitor resistant cell lines Rh41 and MAB391R were determined to have essentially normal copy number; while Rh41-807R was determined to have genomic abnormalities in this region.

FIGS. 8A-B show suppression of PDGFR-α by siRNA reversed the resistance to BMS-754807 in Rh41-807R cells but did not change response in Rh41 cells. Plate A. BMS-754807 dose response curves in Rh41 cells transfected with or without PDGFR-α specific siRNA. Plate B. BMS-754807 dose response curves in Rh41-807R cells transfected with or without PDGFR-α specific siRNA.

FIG. 9 shows expression of PDGFR-α, c-KIT and FGFR2RNA levels are increased in both Rh41-807R cells and Rh41-807R tumors when compared to the corresponding sensitive parental control.

FIG. 10 shows in vivo response to BMS-754807 in Rh41 and Rh41-807R xenografts. 8 tumor bear-mice/group were treated at the indicated dose and schedule.

FIGS. 11A-B show the genes over-expressed in IGF-1R inhibitor resistant cell lines. Plate A. MCF7-807R cell line had expression changes in the components of IGF-1R pathway compared to the sensitive parental line. Plate B. Genes over-expressed in four out of five IGF-1R inhibitor resistant cell lines.

### DETAILED DESCRIPTION OF THE INVENTION

The present invention relates to the identification of markers for predicting resistance to IGF-1R therapy prior to or concurrent with treatment, or for identifying IGF-1R resistance concurrent with treatment, in addition to methods of treating patients with such resistance, in addition to treatment regimens.

Specifically, the present inventors used a rhabdomyosarcoma cell line Rh41 to develop two acquired resistant cell lines: one cell line had resistance to the small molecule IGF-1R inhibitor BMS-754807 (referred to as the “807R” or “Rh41-807R” cell line) and another was resistant to MAB391 (referred to as the “MAB391R” or “R^{h}-MAB391R” cell line), a commercially available IGF-IR neutralizing antibody that competes with IGF-1 binding to IGF-1R and induces receptor degradation in tumor cells (Hailey et al., Mol. Cancer. Ther., 1:1349-1353 (2002)) and is active to inhibit proliferation of Rh41 (Carboni et al., Proceedings of the 100th Annual Meeting of the American Association for Cancer Research, 2009 Apr. 18-22, Denver, Colo., Abstract No. 1742). In addition, tumor xenograft models were developed from the resistant IGF-1R inhibitor cell lines. Gene expression profiling, DNA copy number analysis and signaling pathways were performed on the sensitive parental Rh41, -807R, and -MAb391R cell lines to identify the molecular basis underlying the common mechanisms of acquired resistance to both BMS-754807 and MAB391, as well as unique resistance mechanisms to either drug.

Each resistance model utilized different redundant growth signaling pathways as an escape mechanism. PDGFR-α was amplified, overexpressed and constitutively activated in Rh41-807R cells, and also overexpressed in Rh41-807R tumors. Knockdown of PDGFR-α by siRNA in Rh41-807R re-sensitized the cells to BMS-754807. Synergistic activities were observed when BMS-754807 was combined with PDGFR-α inhibitors in the Rh41-807R models both in vitro and in vivo. On other hand, AXL expression was highly elevated in Rh41-MAB391R but down regulated in Rh41-807R. In addition, BMS-754807 was active in MAB391R cells and able to overcome resistance to the IGF-1R antibody MAB391; However, the converse was not true, i.e., mAb391 did not overcome resistance in the 807R cell line. This suggests that treatment with BMS-754807 may overcome resistance in patients who have developed resistance to treatment with IGF-1R antibody therapies. This study provides insights in acquired resistance to IGF-1R targeted therapies and rationale to prevent or overcome the resistance.

The results demonstrated the synergistic value of combining an IGF-1R inhibitor with a PDGFR-α inhibitor for overcoming IGF-1R inhibitor resistance. PDGFR-α overexpression and activation may drive resistance to BMS-754807, whereas differential resistance mechanisms are involved in resistance to the IGF-1R antibody. The activity of BMS-754807 in the Rh41-mAb391R acquired-resistant model suggests that treatment with BMS-754807 may overcome resistance in patients who have failed treatment with IGF-1R antibodies.

In addition, the inventors also developed additional four IGF-1R resistant cell lines of different tumor types, MCF7, Rh41, Rh1, Geo and SW480 (breast, sarcomas and colon), by inducing acquired resistance to BMS-754807 by stepwise exposure to increasing concentrations of the drug for extended periods of time. Analyses of in vitro drug response, gene expression profiles were performed to characterize the resistant models and the corresponding sensitive parental cells.

Cell line specific, as well as shared molecular, alterations were observed in the different resistant cells using genomic approaches to define mechanisms of resistance to BMS-754807. The resistant models were also tested against multiple IGF-1R inhibitors and showed cross-resistance suggesting common mechanisms of resistance to IGF-1R inhibition.

The inventors believe this is the first report that defines and compares the acquired resistance mechanisms for IGF-1R inhibitors, in general, and for small molecule and anti-IGF-1R antibody based inhibitors, in particular. The results provided important insights into the differentiation of IGF-1R targeted therapies, and have led to the rational development of therapies designed to reverse and/or prevent IGF-1R inhibitor resistance, including, but not limited to combination therapy with EGFR inhibitors and/or PDGFR-α inhibitors.

As is known in the art, BMS-754807 refers to a compound having the following structure (I):

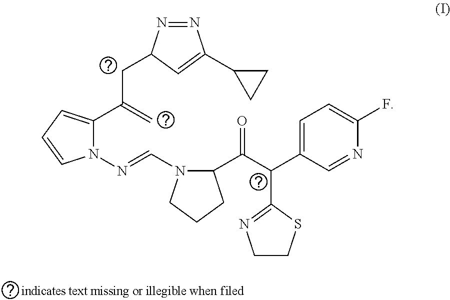

Compound (I) can also be referred to as (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide in accordance with IUPAC nomenclature. Use of the term “(2S)-1-(4-((5-cyclopropyl-1H-pyrazol-3-yl)amino)pyrrolo [2,1-f][1,2,4]triazin-2-yl)-N-(6-fluoro-3-pyridinyl)-2-methyl-2-pyrrolidinecarboxamide” encompasses (unless otherwise indicated) solvates (including hydrates) and polymorphic forms of the compound (I) or its salts, such as the forms of (I) described in U.S. Pat. No. 7,534,792, incorporated herein by reference in its entirety and for all purposes. Pharmaceutical compositions of (2S)-1-(4-((5-cyclopropyl-1H-pyrazol-3-yl)amino) pyrrolo [2,1-f][1,2,4]triazin-2-yl)-N-(6-fluoro-3-pyridinyl)-2-methyl-2-pyrrolidinecarboxamide include all pharmaceutically acceptable compositions comprising (2S)-1-(4-((5-cyclopropyl-1H-pyrazol-3-yl)amino)pyrrolo[2,1-f][1,2,4] triazin-2-yl)-N-(6-fluoro-3-pyridinyl)-2-methyl-2-pyrrolidinecarboxamide and one or more diluents, vehicles and/or excipients One example of a pharmaceutical composition comprising (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide is BMS-754807 (Bristol-Myers Squibb Company). BMS-754807 comprises (2 S)-1-(4-((5-cyclopropyl-1H-pyrazol-3-yl)amino)pyrrolo [2,1-f][1,2,4]triazin-2-yl)-N-(6-fluoro-3-pyridinyl)-2-methyl-2-pyrrolidinecarboxamide as the active ingredient, for IV infusion including inactive ingredients in the form of a diluent.

In one aspect, the IGF-1R antibody is provided in PCT Publication Nos. WO 2005/016970, WO 02/53596, WO 2004/71529, WO 2005/16967, WO 2004/83248, WO 03/106621, WO 03/100008, WO 03/59951, WO 2004/87756, or WO 2005/05635.

In another aspect, the IGF-1R modulator is derived from fibronectin, such as an AdNectin (Adnexus Therapeutics) (See, PCT Publication Nos. WO 00/34784, WO 01/64942, WO 02/32925).

The term “PDGFR-α inhibitor” or “PDGFR-α inhibitor” refers to a small molecule, antibody, siRNA, adnectins, domain antibody, or other molecule capable of inhibiting the expression and/or activity of PDGFR-α, either at the DNA level or protein level. Examples of a PDGFR-α inhibitor include, but are not limited to the following: dovitinib, axitinib, sorafenib and sunitinib.

The term “EGFR inhibitor” refers to a small molecule, antibody, siRNA, adnectins, domain antibody, or other molecule capable of inhibiting the expression and/or activity of EGFR, either at the DNA level or protein level, and either inhibiting the kinase activity of EGFR or the ability of EGF to bind to EGFR, among other activities. Examples of an EGFR inhibitor include the examples provided in the paragraphs that follow in addition to the foregoing: EGFR antibodies that may be chimerized, humanized, fully human, and single chain antibodies derived from the murine antibody 225 described in U.S. Pat. No. 4,943,533.

In another aspect, the EGFR inhibitor is cetuximab (IMC-C225) which is a chimeric (human/mouse) IgG monoclonal antibody, also known under the tradename ERBITUX®. Cetuximab Fab contains the Fab fragment of cetuximab, i.e., the heavy and light chain variable region sequences of murine antibody M225 (U.S. Application No. 2004/0006212, incorporated herein by reference) with human IgG1 C_{H}1 heavy and kappa light chain constant domains. Cetuximab includes all three IgG1 heavy chain constant domains.

In another aspect, the EGFR inhibitor can be selected from the antibodies described in U.S. Pat. Nos. 6,235,883, 5,558,864, and 5,891,996. The EGFR antibody can be, for example, AGX-EGF (Amgen Inc.) (also known as panitumumab) which is a fully human IgG2 monoclonal antibody. The sequence and characterization of ABX-EGF, which was formerly known as clone E7.6.3, is disclosed in U.S. Pat. No. 6,235,883 at column 28, line 62 through column 29, line 36 and FIGS. 29-34, which is incorporated by reference herein. The EGFR antibody can also be, for example, EMD72000 (Merck KGaA), which is a humanized version of the murine EGFR antibody EMD 55900. The EGFR antibody can also be, for example: h-R3 (TheraCIM), which is a humanized EGFR monoclonal antibody; Y10 which is a murine monoclonal antibody raised against a murine homologue of the human EGFRvIII mutation; or MDX-447 (Medarex Inc.).

In addition to the biological molecules discussed above, the EGFR modulators useful in the invention may also be small molecules. Any molecule that is not a biological molecule is considered herein to be a small molecule. Some examples of small molecules include organic compounds, organometallic compounds, salts of organic and organometallic compounds, saccharides, amino acids, and nucleotides. Small molecules further include molecules that would otherwise be considered biological molecules, except their molecular weight is not greater than 450. Thus, small molecules may be lipids, oligosaccharides, oligopeptides, and oligonucleotides and their derivatives, having a molecular weight of 450 or less.

It is emphasized that small molecules can have any molecular weight. They are merely called small molecules because they typically have molecular weights less than 450. Small molecules include compounds that are found in nature as well as synthetic compounds. In one embodiment, the EGFR modulator is a small molecule that inhibits the growth of tumor cells that express EGFR. In another embodiment, the EGFR modulator is a small molecule that inhibits the growth of refractory tumor cells that express EGFR.

Numerous small molecules have been described as being useful to inhibit EGFR.

One example of a small molecule EGFR antagonist is IRESSA® (ZD1939), which is a quinozaline derivative that functions as an ATP-mimetic to inhibit EGFR. See, U.S. Pat. No. 5,616,582; WO 96/33980 at page 4. Another example of a small molecule EGFR antagonist is TARCEVA® (OSI-774), which is a 4-(substituted phenylamino)quinozaline derivative [6,7-bis(2-methoxy-ethoxy)-quinazolin-4-yl]-(3-ethynyl-1-phenyl)amine hydrochloride] EGFR inhibitor. See WO 96/30347 (Pfizer Inc.) at, for example, page 2, line 12 through page 4, line 34 and page 19, lines 14-17. TARCEVA® may function by inhibiting phosphorylation of EGFR and its downstream PI3/Akt and MAP (mitogen activated protein) kinase signal transduction pathways resulting in p27-mediated cell-cycle arrest. See Hidalgo et al., Abstract 281 presented at the 37th Annual Meeting of ASCO, San Francisco, Calif., May 12-15, 2001.

Other small molecules are also reported to inhibit EGFR, many of which are thought to be specific to the tyrosine kinase domain of an EGFR. Some examples of such small molecule EGFR antagonists are described in WO 91/116051, WO 96/30347, WO 96/33980, WO 97/27199. WO 97/30034, WO 97/42187, WO 97/49688, WO 98/33798, WO 00/18761, and WO 00/31048. Examples of specific small molecule EGFR antagonists include CI-1033 (Pfizer Inc.), which is a quinozaline (N-[4-(3-chloro-4-fluoro-phenylamino)-7-(3-mprpholin-4-yl-propoxy)-quinazolin-6-yl]-acrylamide) inhibitor of tyrosine kinases, particularly EGFR and is described in WO 00/31048 at page 8, lines 22-6; PKI166 (Novartis), which is a pyrrolopyrimidine inhibitor of EGFR and is described in WO 97/27199 at pages 10-12; GW2016 (GlaxoSmithKline), which is an inhibitor of EGFR and HER2; EKB569 (Wyeth), which is reported to inhibit the growth of tumor cells that overexpress EGFR or HER2 in vitro and in vivo; AG-1478 (Tryphostin), which is a quinazoline small molecule that inhibits signaling from both EGFR and erbB-2; AG-1478 (Sugen), which is a bisubstrate inhibitor that also inhibits protein kinase CK2; PD 153035 (Parke-Davis) which is reported to inhibit EGFR kinase activity and tumor growth, induce apoptosis in cells in culture, and enhance the cytotoxicity of cytotoxic chemotherapeutic agents; SPM-924 (Schwarz Pharma), which is a tyrosine kinase inhibitor targeted for treatment of prostrate cancer; CP-546,989 (OSI Pharmaceuticals), which is reportedly an inhibitor of angiogenesis for treatment of solid tumors; ADL-681, which is a EGFR kinase inhibitor targeted for treatment of cancer; PD 158780, which is a pyridopyrimidine that is reported to inhibit the tumor growth rate of A4431 xenografts in mice; CP-358,774, which is a quinzoline that is reported to inhibit autophosphorylation in HN5 xenografts in mice; ZD1839, which is a quinzoline that is reported to have antitumor activity in mouse xenograft models including vulvar, NSCLC, prostrate, ovarian, and colorectal cancers; CGP 59326A, which is a pyrrolopyrimidine that is reported to inhibit growth of EGFR-positive xenografts in mice; PD 165557 (Pfizer); CGP54211 and CGP53353 (Novartis), which are dianilnophthalimides. Naturally derived EGFR tyrosine kinase inhibitors include genistein, herbimycin A, quercetin, and erbstatin.

Further small molecules reported to inhibit EGFR and that are therefore within the scope of the present invention are tricyclic compounds such as the compounds described in U.S. Pat. No. 5,679,683; quinazoline derivatives such as the derivatives described in U.S. Pat. No. 5,616,582; and indole compounds such as the compounds described in U.S. Pat. No. 5,196,446.

Further small molecules reported to inhibit EGFR and that are therefore within the scope of the present invention are styryl substituted heteroaryl compounds such as the compounds described in U.S. Pat. No. 5,656,655. The heteroaryl group is a monocyclic ring with one or two heteroatoms, or a bicyclic ring with 1 to about 4 heteroatoms, the compound being optionally substituted or polysubstituted.

Further small molecules reported to inhibit EGFR and that are therefore within the scope of the present invention are bis mono and/or bicyclic aryl heteroaryl, carbocyclic, and heterocarbocyclic compounds described in U.S. Pat. No. 5,646,153.

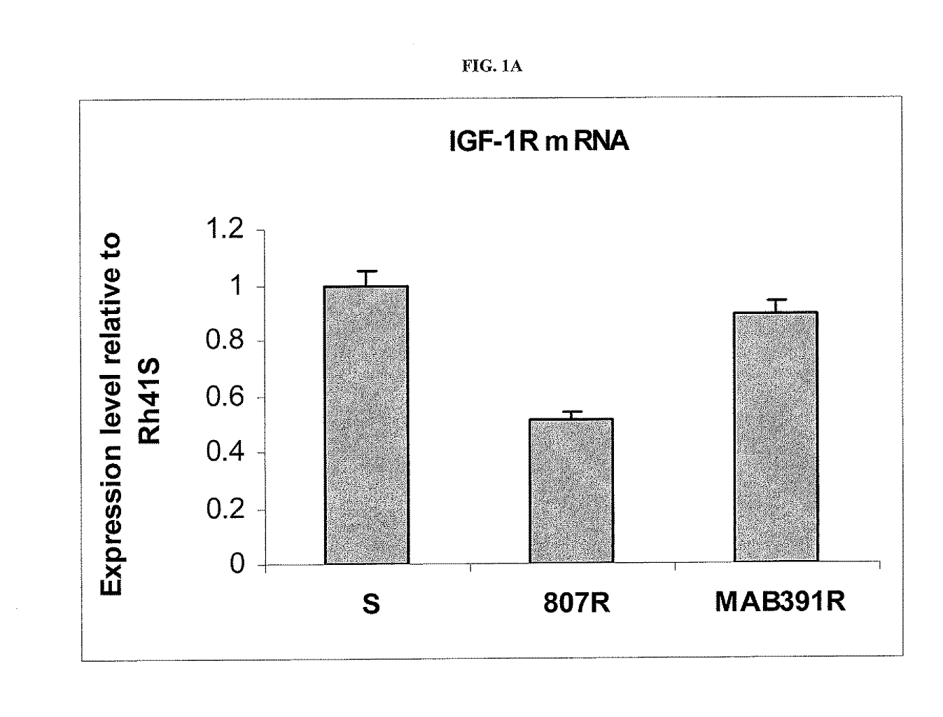

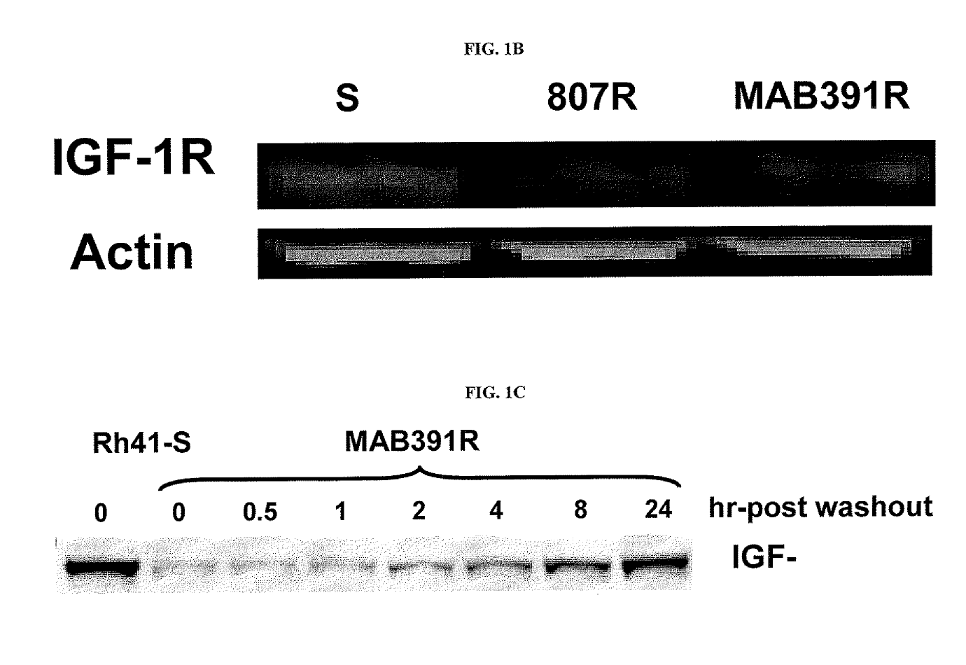

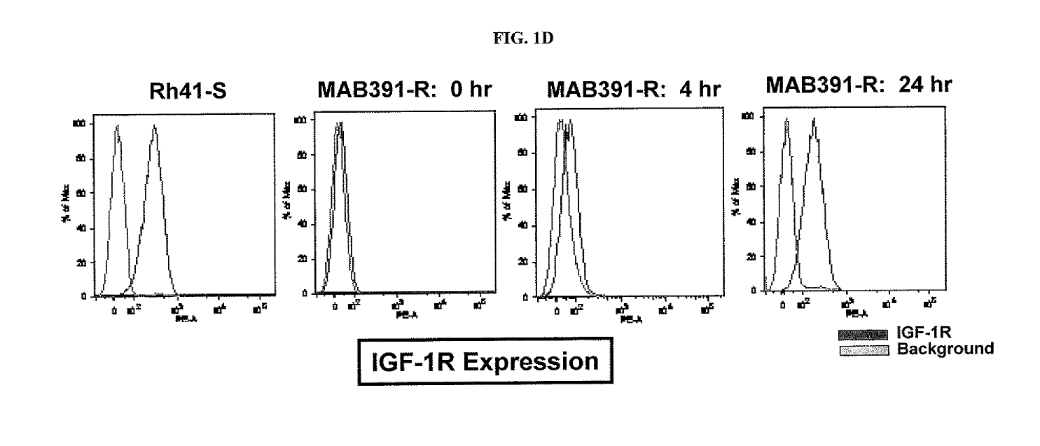

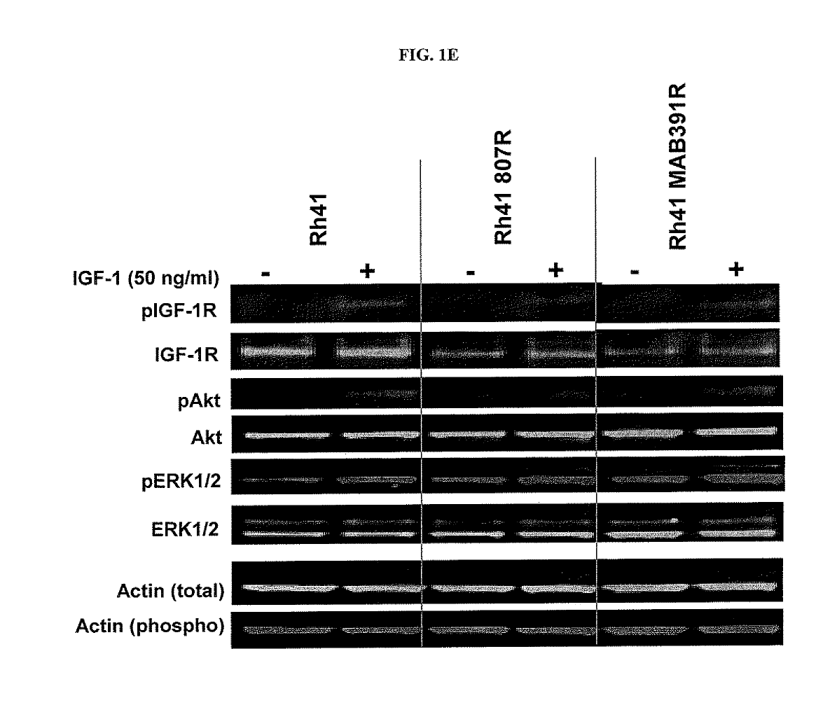

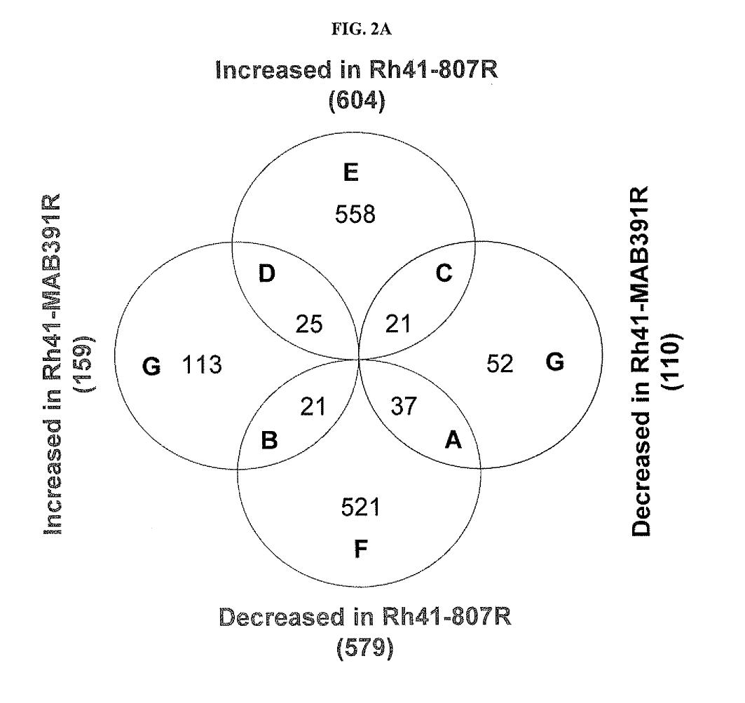

Further small molecules reported to inhibit EGFR and that are therefore within the scope of the present invention is the compound provided FIG. 1 of Fry et al., Science, 265:1093-1095 (1994) that inhibits EGFR.

Further small molecules reported to inhibit EGFR and that are therefore within the scope of the present invention are tyrphostins that inhibit EGFR/HER1 and HER 2, particularly those in Tables I, II, III, and IV described in Osherov et al., J. Biol. Chem., 268(15):11134-11142 (1993).

Further small molecules reported to inhibit EGFR and that are therefore within the scope of the present invention is a compound identified as PD166285 that inhibits the EGFR, PDGFR, and FGFR families of receptors. PD166285 is identified as 6-(2,6-dichlorophenyl)-2-(4-(2-diethylaminoethyoxy)phenylamino)-8-methyl-8H-pyrido(2,3-d)pyrimidin-7-one having the structure shown in FIG. 1 on page 1436 of Panek et al., J. Pharmacol. Exp. Ther., 283:1433-1444 (1997).

In addition to the biological molecules discussed above, the IGF1R modulators useful in the invention may also be small molecules. Any molecule that is not a biological molecule is considered herein to be a small molecule. Some examples of small molecules include organic compounds, organometallic compounds, salts of organic and organometallic compounds, saccharides, amino acids, and nucleotides. Small molecules further include molecules that would otherwise be considered biological molecules, except their molecular weight is not greater than 450. Thus, small molecules may be lipids, oligosaccharides, oligopeptides, and oligonucleotides and their derivatives, having a molecular weight of 450 or less.

It is emphasized that small molecules can have any molecular weight. They are merely called small molecules because they typically have molecular weights less than 450. Small molecules include compounds that are found in nature as well as synthetic compounds. In one embodiment, the IGF1R modulator is a small molecule that inhibits the growth of tumor cells that express IGF1R. In another embodiment, the IGF1R modulator is a small molecule that inhibits the growth of refractory tumor cells that express IGF1R.

Numerous small molecules have been described as being useful to inhibit IGF1R.

In one aspect, the IGF1R modulator is selected from PCT Publication Nos. WO 02/79192, WO 2004/30620, WO 2004/31401 WO 2004/63151, and WO 2005/21510, and from U.S. Provisional Application Nos. 60/819,171, 60/870,872, 60/883,601, and 60/912,446.

In another aspect, the IGF-1R modulator is selected from (S)-4-(2-(3-chlorophenyl)-2-hydroxyethylamino)-3-(4-methyl-6-morpholino-1H-benzo[d]imidazol-2-yl)-pyridin-2 (1-H)-one and (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide.

In another aspect, the IGF-1R modulator is selected from XL-228 (Exelixis), AEW-541 (Novartis), and OSI-906 (OSI).

The phrase “microtubulin modulating agent” is meant to refer to agents that either stabilize microtubulin or destabilize microtubulin synthesis and/or polymerization.

Microtubulin modulatory agents either agonize or inhibit a cells ability to maintain proper microtubulin assemblies. In the case of paclitaxel (marketed as TAXOL®) causes mitotic abnormalities and arrest, and promotes microtubule assembly into calcium-stable aggregated structures resulting in inhibition of cell replication.

Epothilones mimic the biological effects of TAXOL®, (Bollag et al., Cancer Res., 55:2325-2333 (1995), and in competition studies act as competitive inhibitors of TAXOL® binding to microtubules. However, epothilones enjoy a significant advantage over TAXOL® in that epothilones exhibit a much lower drop in potency compared to TAXOL® against a multiple drug-resistant cell line (Bollag et al. (1995)). Furthermore, epothilones are considerably less efficiently exported from the cells by P-glycoprotein than is TAXOL® (Gerth et al. (1996)).

Ixabepilone is a semi-synthetic lactam analogue of patupilone that binds to tubulin and promotes tubulin polymerization and microtubule stabilization, thereby arresting cells in the G2/M phase of the cell cycle and inducing tumor cell apoptosis.

Thus, in one embodiment, the therapeutic method of the invention comprises the administration of an epothilone in combination with an IGF-1R inhibitor.

Combinations of an IGF-1R inhibitor with another agent is contemplated by the present invention, and may include the addition of an anti-proliferative cytotoxic agent. Classes of compounds that may be used as anti-proliferative cytotoxic agents include the following:

co-stimulatory modulating agents including, without limitation, CTLA4 antagonists, ipilimumab, agatolimod, belatacept, blinatumomab, CD40 ligand, anti-B7-1 antibody, anti-B7-2 antibody, anti-B7-H4 antibody, AG4263, eritoran, anti-OX40 antibody, ISF-154, and SGN-70;

alkylating agents (including, without limitation, nitrogen mustards, ethylenimine derivatives, alkyl sulfonates, nitrosoureas and triazenes): Uracil mustard, Chlormethine, Cyclophosphamide (CYTOXAN®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide;

antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine; and natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as TAXOL®), Mithramycin, Deoxyco-formycin, Mitomycin-C, L-Asparaginase, Interferons (especially IFN-a), Etoposide, and Teniposide.

Other anti-proliferative cytotoxic agents contemplated by the present invention are navelbene, CPT-11, anastrazole, letrazole, capecitabine, reloxafine, cyclophosphamide, ifosamide, and droloxafine.

The present invention also encompasses a pharmaceutical composition useful in the treatment of cancer, comprising the administration of a therapeutically effective amount of an IGF-1R inhibitor, either alone or in combination with another agent, with or without pharmaceutically acceptable carriers or diluents. The compositions of the present invention may further comprise one or more pharmaceutically acceptable additional ingredient(s) such as alum, stabilizers, antimicrobial agents, buffers, coloring agents, flavoring agents, adjuvants, and the like. The IGF-1R inhibitor, or analogs thereof compounds, PDFGR-a inhibitor, or analogs thereof compounds, or EGFR-inhibitors, or analogs thereof compounds, antineoplastic agents, and compositions of the present invention may be administered orally or parenterally including the intravenous, intramuscular, intraperitoneal, subcutaneous, rectal and topical routes of administration.

For oral use, the antineoplastic agents, IGF-1R inhibitor, PDGFR-α inhibitor, and/or EGFR inhibitor or analogs thereof compounds and compositions of this invention may be administered, for example, in the form of tablets or capsules, powders, dispersible granules, or cachets, or as aqueous solutions or suspensions. In the case of tablets for oral use, carriers which are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc, and sugar. When aqueous suspensions are used for oral administration, emulsifying and/or suspending agents are commonly added.

In addition, sweetening and/or flavoring agents may be added to the oral compositions. For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled in order to render the preparation isotonic.

For preparing suppositories according to the invention, a low melting wax such as a mixture of fatty acid glycerides or cocoa butter is first melted, and the active ingredient is dispersed homogeneously in the wax, for example by stirring. The molten homogeneous mixture is then poured into conveniently sized molds and allowed to cool and thereby solidify.

Liquid preparations include solutions, suspensions and emulsions. Such preparations are exemplified by water or water/propylene glycol solutions for parenteral injection. Liquid preparations may also include solutions for intranasal administration.

Aerosol preparations suitable for inhalation may include solutions and solids in powder form, which may be in combination with a pharmaceutically acceptable carrier, such as an inert compressed gas.

Also included are solid preparations which are intended for conversion, shortly before use, to liquid preparations for either oral or parenteral administration. Such liquid forms include solutions, suspensions and emulsions.

The IGF-1R inhibitor, PDGFR-α inhibitor, and/or EGFR inhibitor or analogs thereof, as well as anti-neoplastic agents, described herein may also be delivered transdermally. The transdermal compositions can take the form of creams, lotions, aerosols and/or emulsions and can be included in a transdermal patch of the matrix or reservoir type as are conventional in the art for this purpose.

The combinations of the present invention may also be used in conjunction with other well known therapies that are selected for their particular usefulness against the condition that is being treated.

If formulated as a fixed dose, the active ingredient(s) of the microtubulin-stabilizing agents, or combination compositions, of this invention are employed within the dosage ranges described below. Alternatively, the anti-CTLA4 agent, and IGF-1R inhibitor, PDGFR-α inhibitor, and/or EGFR inhibitor or analogs thereof compounds may be administered separately in the dosage ranges described below. In a preferred embodiment of the present invention, the anti-CTLA4 agent is administered in the dosage range described below following or simultaneously with administration of the IGF-1R inhibitor, PDGFR-α inhibitor, and/or EGFR inhibitor or analogs thereof compound in the dosage range described below.

The following sets forth preferred therapeutic combinations and exemplary dosages for use in the methods of the present invention. Where “Compound of Formula II” appears, any of the variations of Formula II or Formula III set forth herein are contemplated for use in the chemotherapeutic combinations. Preferably, Compound I or Compound 4 is employed.

| | Dosage | |
|---|---|---|
| Therapeutic Combination | mg/m² (per dose) | |
| Compound of Formula I (BMS-754807) | 1-500 | mg/m² |
| Compound of Formula I (BMS-754807) + | 1-500 | mg/m² |
| PDGFR-α Inhibitor | 0.1-25 | mg/kg |
| Compound of Formula I (BMS-754807) + | 1-500 | mg/m² |
| EGFR Inhibitor | 0.1-25 | mg/kg |
| Anti-IGF-1R Antibody | 1-500 | mg/m² |
| Anti-IGF-1R Antibody + | 1-500 | mg/m² |
| PDGFR-α Inhibitor | 0.1-25 | mg/kg |
| Anti-IGF-1R Antibody + | 0.1-100 | mg/m² |
| EGFR Inhibitor | 0.1-25 | mg/kg |

While this table provides exemplary dosage ranges of the IGF-1R inhibitors and certain anticancer agents of the invention, when formulating the pharmaceutical compositions of the invention the clinician may utilize preferred dosages as warranted by the condition of the patient being treated. For example, the compound of Formula I may preferably be administered at about 4, 10, 20, 30, 50, 70, 100, 130, 160, or 200 mg/m² daily.

The anti-IGF-1R antibody may preferably be administered at about 0.3-10 mg/kg, or the maximum tolerated dose. In an embodiment of the invention, a dosage of IGF-1R antibody is administered about every three weeks. Alternatively, the IGF-1R antibody may be administered by an escalating dosage regimen including administering a first dosage of IGF-1R antibody at about 3 mg/kg, a second dosage of IGF-1R antibody at about 5 mg/kg, and a third dosage of IGF-1R antibody at about 9 mg/kg.

In another specific embodiment, the escalating dosage regimen includes administering a first dosage of IGF-1R antibody at about 5 mg/kg and a second dosage of IGF-1R antibody at about 9 mg/kg.

Further, the present invention provides an escalating dosage regimen, which includes administering an increasing dosage of IGF-1R antibody about every six weeks.

In an aspect of the present invention, a stepwise escalating dosage regimen is provided, which includes administering a first IGF-1R antibody dosage of about 3 mg/kg, a second IGF-1R antibody dosage of about 3 mg/kg, a third IGF-1R antibody dosage of about 5 mg/kg, a fourth IGF-1R antibody dosage of about 5 mg/kg, and a fifth IGF-1R antibody dosage of about 9 mg/kg. In another aspect of the present invention, a stepwise escalating dosage regimen is provided, which includes administering a first dosage of 5 mg/kg, a second dosage of 5 mg/kg, and a third dosage of 9 mg/kg.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In accordance with the diagnostic methods of the present invention, a treatment regimen may be assigned according to whether the patient is predicted to have a favorable or a less than favorable response. For those individuals predicted to have a favorable response, an ordinary IGF-1R inhibitor dosing regiment may be administered. However, for those patients who are predicted to have a lower likelihood of achieving a favorable response (i.e., those individuals having elevated expression of AXL, EGFR, IGFBP, PDGFR-α, or those individuals having decreased expression of IGF-1R), an increased dosage of an IGF-1R inhibitor or an IGF-1R inhibitor in combination with other therapy may be warranted. Such an increased level of a therapeutically-effective dose of an IGF-1R inhibitor or an IGF-1R inhibitor in combination with other therapy for an individual identified as being less likely to have a favorable response can be, for example, about 5%, 10%, 15%, 20%, 25%, 30%, 35%, 40%, 45%, 50%, 55%, 60%, 65%, 75%, 80%, 85%, 90%, or 95% higher, or 1.5-, 2-, 2.5-, 3-, 3.5-, 4-, 4,5-, or even 5-fold higher than the prescribed or typical dose, as may be the case.

Alternatively, for those patients who are predicted to have a lower likelihood of achieving a favorable response (i.e., those individuals having elevated expression of AXL, EGFR, IGFBP, PDGFR-α, or those individuals having decreased expression of IGF-1R), an increased frequency dosing regimen of an IGF-1R inhibitor, and/or an IGF-1R inhibitor in combination with other therapy may be warranted. Such an increased frequency dosing regimen of a therapeutically-effective dose of an IGF-1R inhibitor and/or an IGF-1R inhibitor in combination with other therapy for an individual identified as being less likely to have a favorable response can be, for example, about per once week, about once per 6 days, about once per 5 days, about once per 4 days, about once per 3 days, about once per 3 days, about once per 2 days, about once per day, about twice per day, about three per day, about four per day, or about 5%, 10%, 15%, 20%, 25%, 30%, 35%, 40%, 45%, 50%, 55%, 60%, 65%, 75%, 80%, 85%, 90%, or 95% higher, or 1.5-, 2-, 2.5-, 3-, 3.5-, 4-, 4,5-, or even 5-fold higher dosing frequency than the prescribed or typical dose, as may be the case.

In the instance where it may be desirable to administer a microtubulin stabilizing agent, such as paclitaxel or carboplatin, to the IGF-1R treatment, or to the combination treatment of and IGF-1R inhibitor with a PDGFR-α inhibitor and/or EGFR inhibitor, paclitaxel may be administered about 200 mg/m², Day 1 of a 21-day cycle via IV, whereas carboplatin may be administered about 6 mg/ml/min, Day 1 of a 21-day cycle via IV.

In the instance where it may be desirable to administer a HER2 inhibitor, such as HERCEPTIN®, to the IGF-1R treatment, or to the combination treatment of and IGF-1R inhibitor with a PDGFR-α inhibitor and/or EGFR inhibitor, HERCEPTIN® may be administered about 4 mg/kg Day 1 loading dose, 2 mg/kg once weekly via IV.

Certain cancers can be treated effectively with compounds of IGF-1R inhibitor, PDGFR-α inhibitor, and/or EGFR inhibitor and a one or more anti-CTLA4 agents. Such triple and quadruple combinations can provide greater efficacy. When used in such triple and quadruple combinations the dosages set forth above can be utilized.

When employing the methods or compositions of the present invention, other agents used in the modulation of tumor growth or metastasis in a clinical setting, such as antiemetics, can also be administered as desired.

The present invention encompasses a method for the synergistic treatment of cancer comprising the administration of a synergistic combination of an IGF-1R inhibitor and PDGFR-α inhibitor wherein said administration is performed simultaneously or sequentially. Thus, while a pharmaceutical formulation comprising an IGF-1R inhibitor in combination with a PDGFR-α inhibitor may be advantageous for administering the combination for one particular treatment, prior administration of the PDGFR-α inhibitor may be advantageous in another treatment. It is also understood that the instant combination of IGF-1R inhibitor and PDGFR-α inhibitor, may be used in conjunction with other methods of treating cancer (preferably cancerous tumors) including, but not limited to, radiation therapy and surgery. It is further understood that a cytostatic or quiescent agent, if any, may be administered sequentially or simultaneously with any or all of the other synergistic therapies.

The combinations of the instant invention may also be co-administered with other well known therapeutic agents that are selected for their particular usefulness against the condition that is being treated. Combinations of the instant invention may alternatively be used sequentially with known pharmaceutically acceptable agent(s) when a multiple combination formulation is inappropriate.

The chemotherapeutic agent(s) and/or radiation therapy can be administered according to therapeutic protocols well known in the art. It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

In the methods of this invention, a compound of Formula I or an IGF-1R inhibitor is administered simultaneously or sequentially with a PDGFR-α inhibitor and/or an EGFR inhibitor. Thus, it is not necessary that the PDGFR-α inhibitor and/or an EGFR inhibitor and IGF-1R inhibitor, be administered simultaneously or essentially simultaneously. The advantage of a simultaneous or essentially simultaneous administration is well within the determination of the skilled clinician.

Also, in general, the IGF-1R inhibitor, PDGFR-α inhibitor, and/or EGFR inhibitor, do not have to be administered in the same pharmaceutical composition, and may, because of different physical and chemical characteristics, have to be administered by different routes. For example, the anti-IGF-1R antibody may be administered intravenously to generate and maintain good blood levels thereof, while the PDGFR-α inhibitor and/or an EGFR inhibitor may also be administered intravenously. Alternatively, the compound of Formula I or an IGF-1R inhibitor may be administered orally to generate and maintain good blood levels thereof, while the PDGFR-α inhibitor and/or an EGFR inhibitor may be administered intravenously. Alternatively, the compound of Formula I or an IGF-1R antibody may be administered intravenously to generate and maintain good blood levels thereof, while the PDGFR-α inhibitor and/or an EGFR inhibitor may also be administered orally. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice an IGF-1R inhibitor, PDGFR-α inhibitor, and/or EGFR inhibitor or analogs thereof will depend upon the diagnosis of the attending physicians and their judgment of the condition of the patient and the appropriate treatment protocol.

If the compound of Formula I or an anti-IGF-1R antibody and the PDGFR-α inhibitor, and/or EGFR inhibitor are not administered simultaneously or essentially simultaneously, then the initial order of administration of the compound of Formula I or IGF-1R inhibitor, PDGFR-α inhibitor, and/or EGFR inhibitor may be varied. Examples of different orders of administration are outlined elsewhere herein. The alternate administrations outlined herein may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the PDGFR-α inhibitor, and/or EGFR inhibitor may be administered initially. The treatment is then continued with the administration of the compound of formula I or an IGF-1R inhibitor or analogs thereof and optionally followed by administration of a cytostatic agent, if desired, until the treatment protocol is complete. Alternatively, the administration of the compound of Formula I or an IGF-1R inhibitor or analogs thereof and optionally followed by administration of a cytostatic agent may be administered initially. The treatment is then continued with the administration of the PDGFR-α inhibitor, and/or EGFR inhibitor, until the treatment protocol is complete.

Thus, in accordance with experience and knowledge, the practicing physician can modify each protocol for the administration of a component (therapeutic agent—i.e., compound of IGF-1R inhibitor, PDGFR-α inhibitor, and/or EGFR inhibitor or analogs thereof, anti-IGF-1R antibody agent(s)) of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Thus, the present invention provides methods for the treatment of a variety of cancers, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, philadelphia chromosome positive acute lymphoblastic leukemia (Ph+ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof.

Most preferably, the invention is used to treat accelerated or metastatic cancers of the breast and/or lung.

### Discussion

It has become increasingly evident that multiple resistance mechanisms compromise the successful clinical application of inhibitors targeting oncogenic tyrosine kinases particularly in advanced solid tumors (Jänne et al., Nat. Rev. Drug Discov., 8(9):709-723 (2009); Engelman et al., Curr. Opin. Genet. Dev., 18:73-79 (2008)). As both IGF-1R antibody and small molecular inhibitors are currently in clinical testing, it is critically important to understand the mechanisms of resistance to IGF-1R inhibitors, so the strategy for rationally combining therapies could be defined to possibly reverse or prevent the resistance. The major aim of this study was to identify the mechanisms of acquired resistance to IGF-1R targeted therapies. For this purpose, the cell lines with acquired resistance either to BMS-754807 or to IGF-1R antibody MAB391 were developed from Rh41 rhabdomyosarcoma cancer cells in vitro to compare and define the commonality and difference in resistance mechanisms to small molecular inhibitor and to anti-IGF-1R antibody.

There are two fundamental forms of drug resistance: de novo resistance, which refers to the failure to initial treatment of a drug, and acquired resistance, which refers to the relapse on a drug treatment after initial response. Accumulating evidence suggests that similar molecular mechanisms could underlie both forms of resistance (Jänne et al., Nat. Rev. Drug Discov., 8(9):709-723 (2009); Engelman et al., Curr. Opin. Genet. Dev., 18:73-79 (2008)). As the inventors previously reported, IGF-1R expression level is associated with response to IGF-1R inhibitors in sarcoma cell line panel; and lower expression of IGF-1R was seen in more resistant cell lines (Huang et al., Cancer Res., 69(1):161-170 (Jan. 1, 2009)). This was further confirmed by a recent report for an anti-IGFIR-targeting antibody in breast and colon cell lines (Zha et al., Mol. Cancer. Ther., 8(8):2110-2121 (August 2009)). In the present study, the present inventors observed down-regulation of IGF-1R in the models of acquired resistance to either small molecule inhibitors, such as BMS-754807, or to antibody-based inhibitors such as MAB391 (FIGS. 1A, 1B) although via different mechanisms of action. In MAB391R cells, the IGF-1R RNA expression level didn't change, but protein levels decreased significantly (FIGS. 1A, 1B) probably through the receptor internalization. Interestingly, when the antibody was removed, the IGF-1R protein level restored quickly 2 to 4 hrs after withdraw of MAB391 and was completely restored after 24 hrs. However, in 807R cells, decreased levels of IGF-1R was through down regulation of RNA transcription and such down regulation persisted even when the drug was removed (FIG. 1A). It is possible that low IGF-1R expression leads to fewer numbers of receptor on the membrane, and consequently a weak pathway activity. This could possibly serve as one of the resistance mechanisms for both de novo and acquired resistance to IGF-1R inhibitors.

Alterations of drug transporters or drug-metabolizing pathways may block the bioavailability of the tyrosine kinase inhibitors, thus leading to drug-dependent resistance. The ABC (ATP binding cassette) family of membrane transport proteins, including best-known mediators of resistance MDR1 and MRP1, actively extrude many types of drugs from cancer cells, thereby conferring resistance to those agents (Gottesman et al., Nat. Rev. Cancer, 2:48-58 (2002)). To determine if ABC family members were involved in resistance to IGF-1R inhibitors, the present inventors compared 807R or MAB391R to the parental Rh41 cells in regarding expression of all ABC family members, and noticed none of them had a significant difference between these cell lines, thus they were not contributing to the resistance mechanisms in these models.

Furthermore, our data indicated that 807R cells resistant to IGF-1R inhibitors are still sensitive to the cytotoxic agents investigated (Table 2). It is well known that an increased level of IGF-IR signaling reduces the sensitivity to chemotherapeutic drugs in vitro and in vivo, and attenuation of IGF-1R activity increases the efficiency of them (Gooch et al., Breast Cancer Res. Treat., 56:1-10 (1999)). Apparently, 807R cells still retained the sensitizing effect of IGF-1R inhibition, suggesting the mechanism of resistance to IGF-1R inhibitors didn't impact the responsiveness to cytotoxic agents.

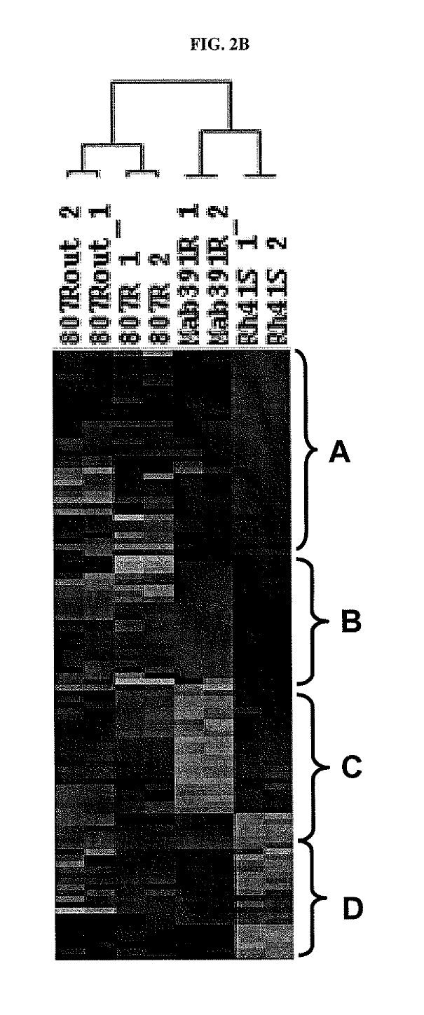

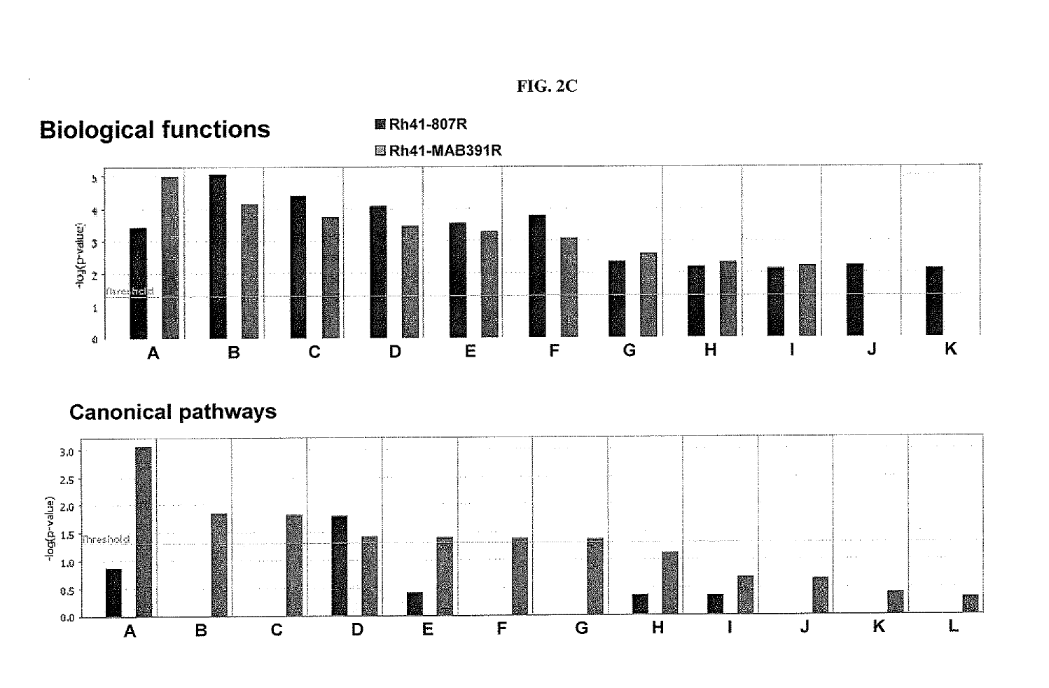

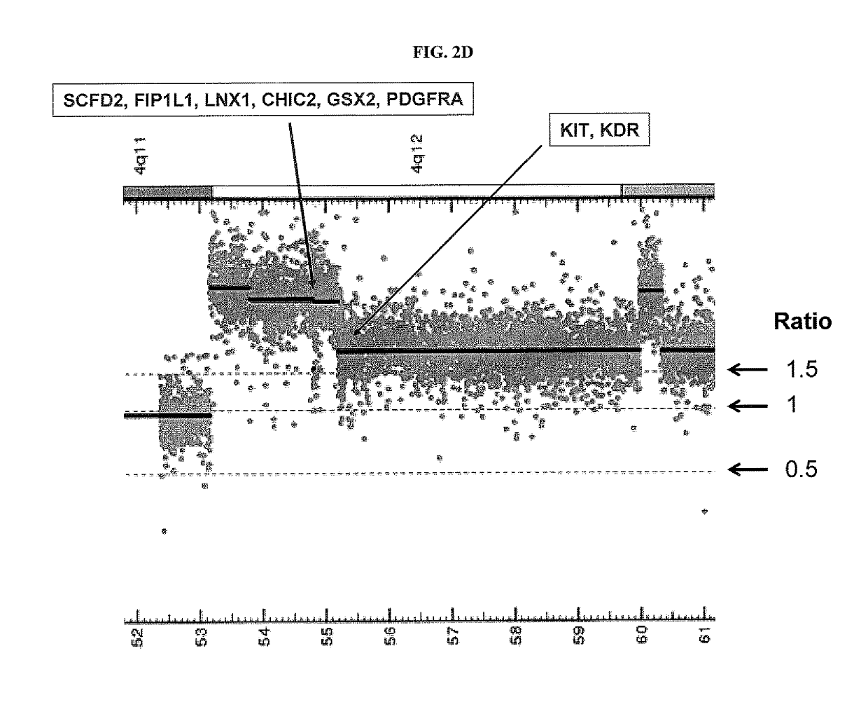

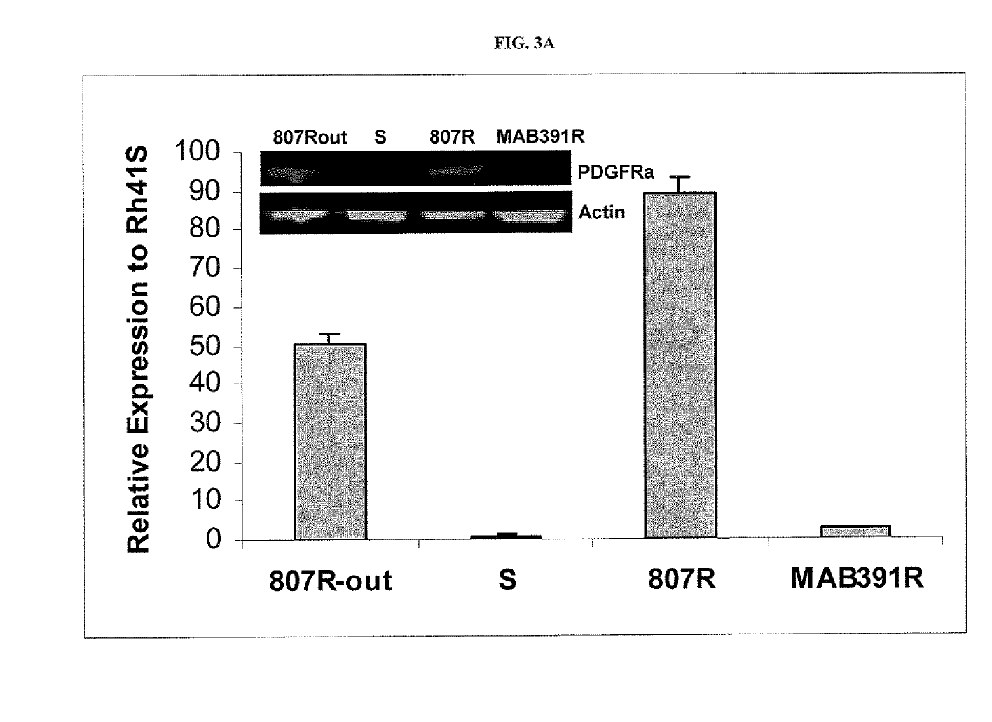

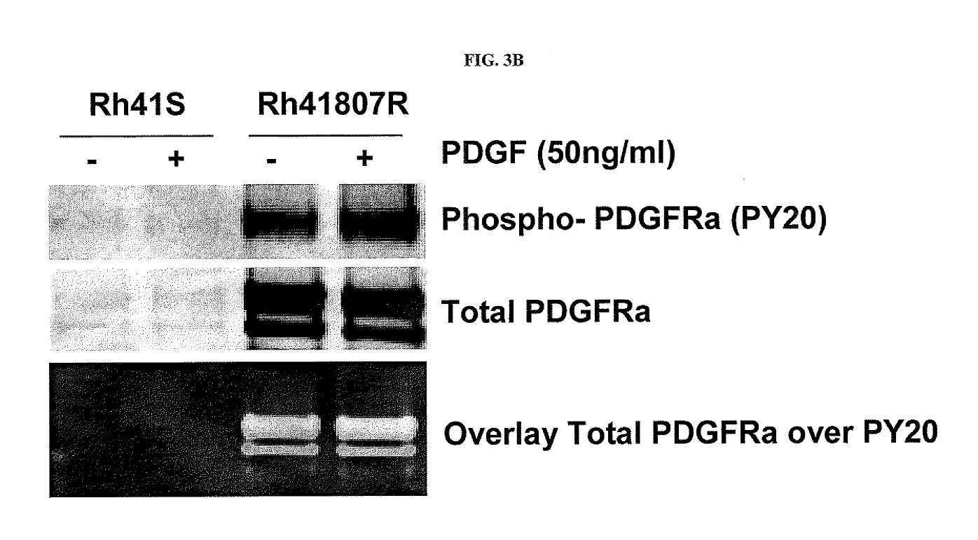

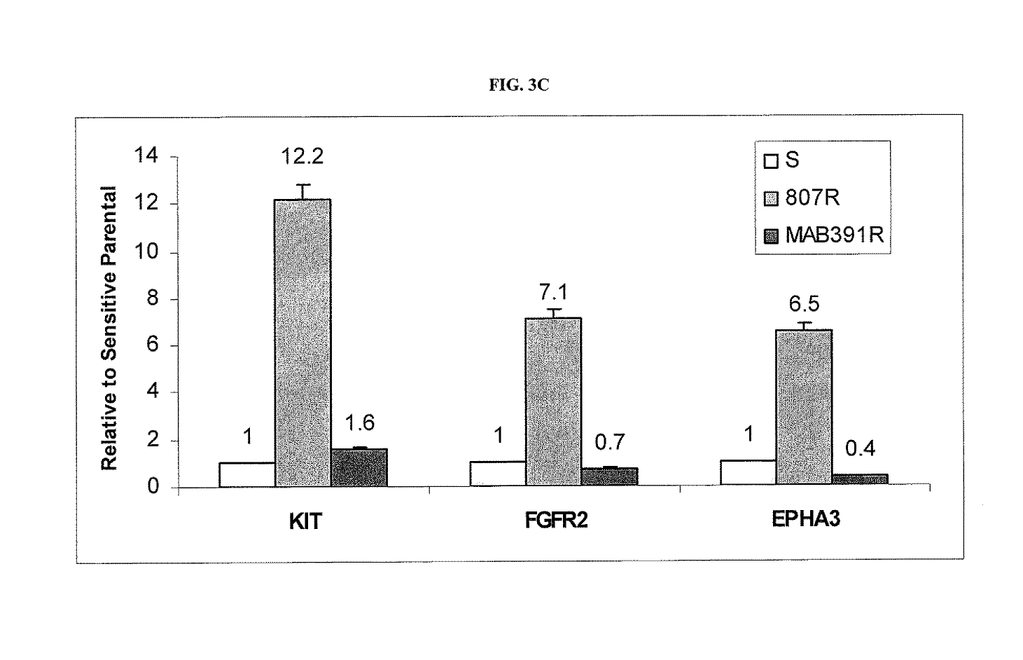

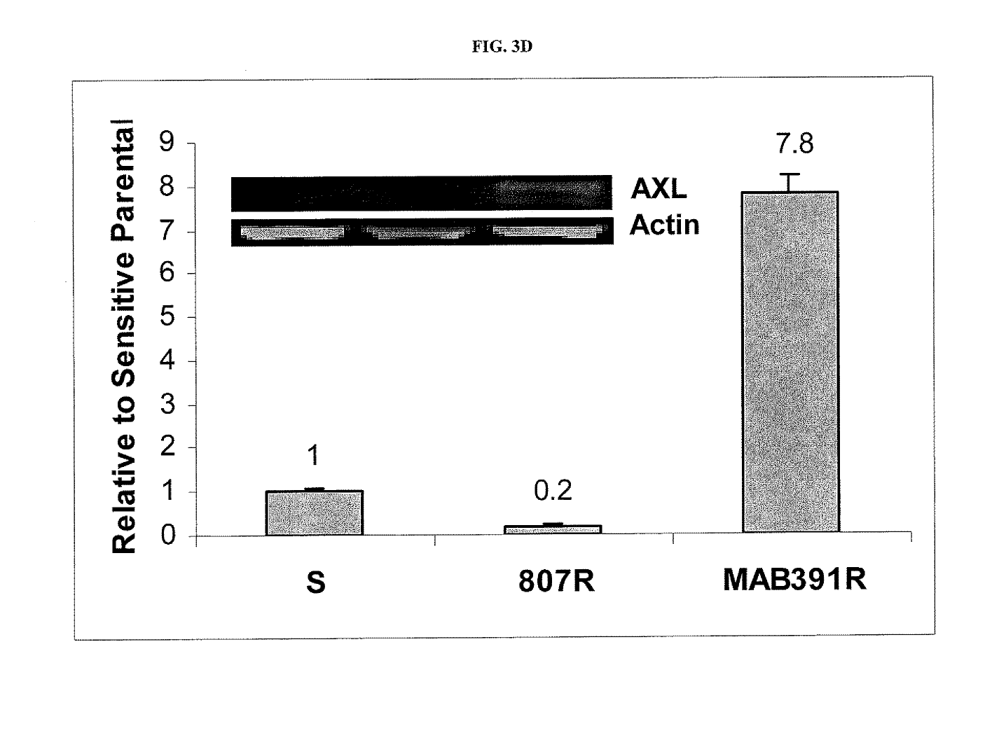

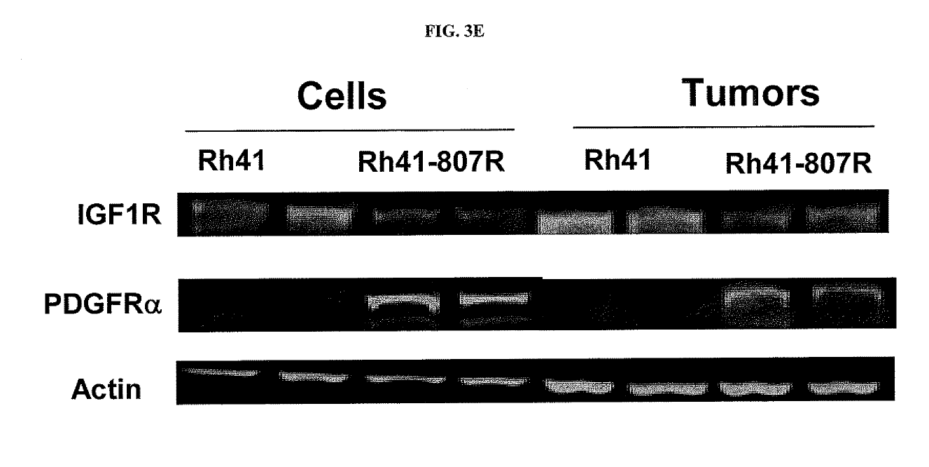

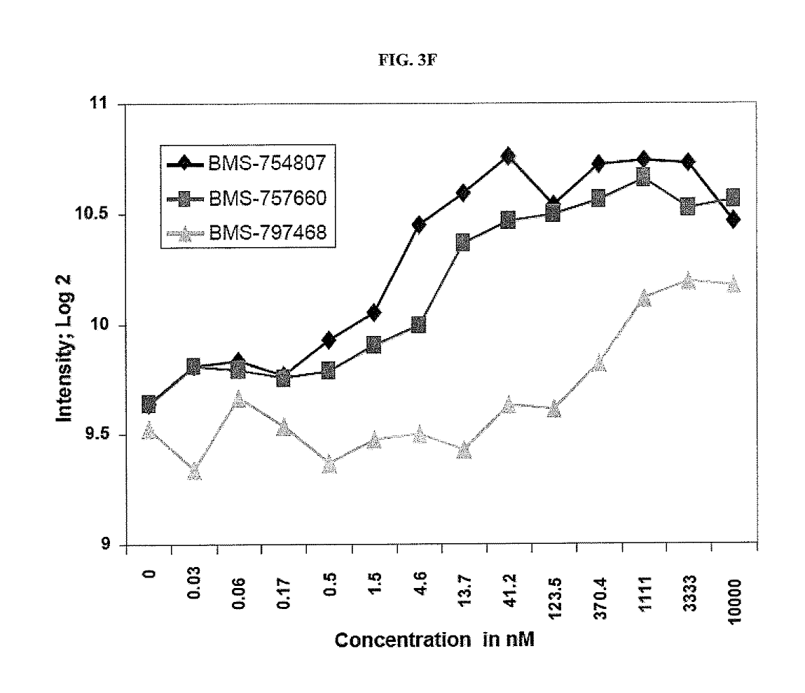

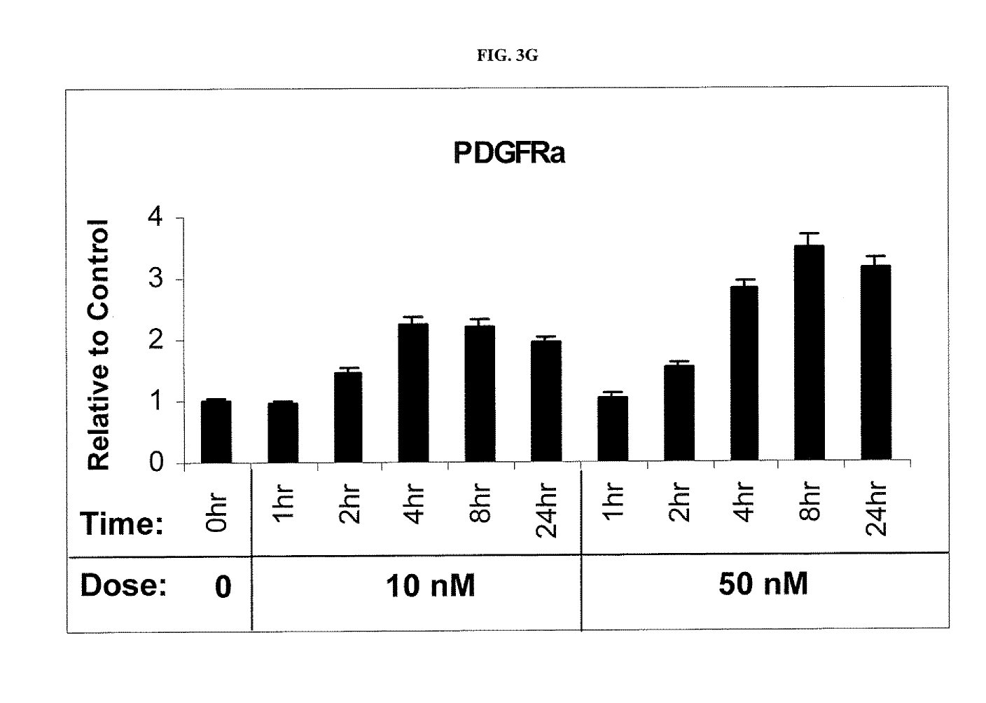

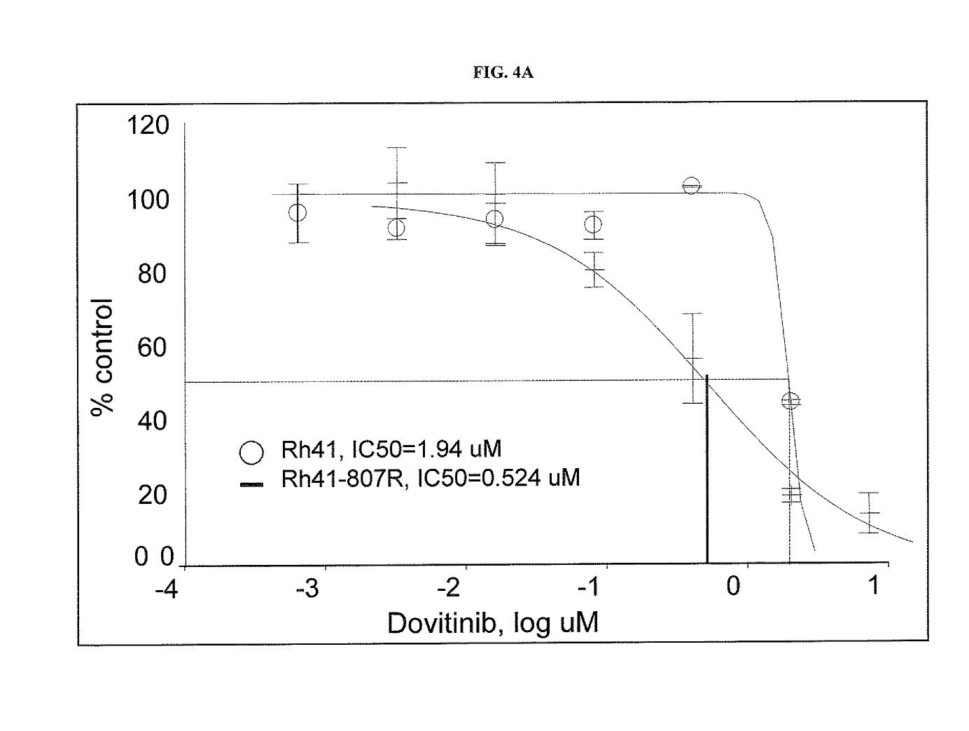

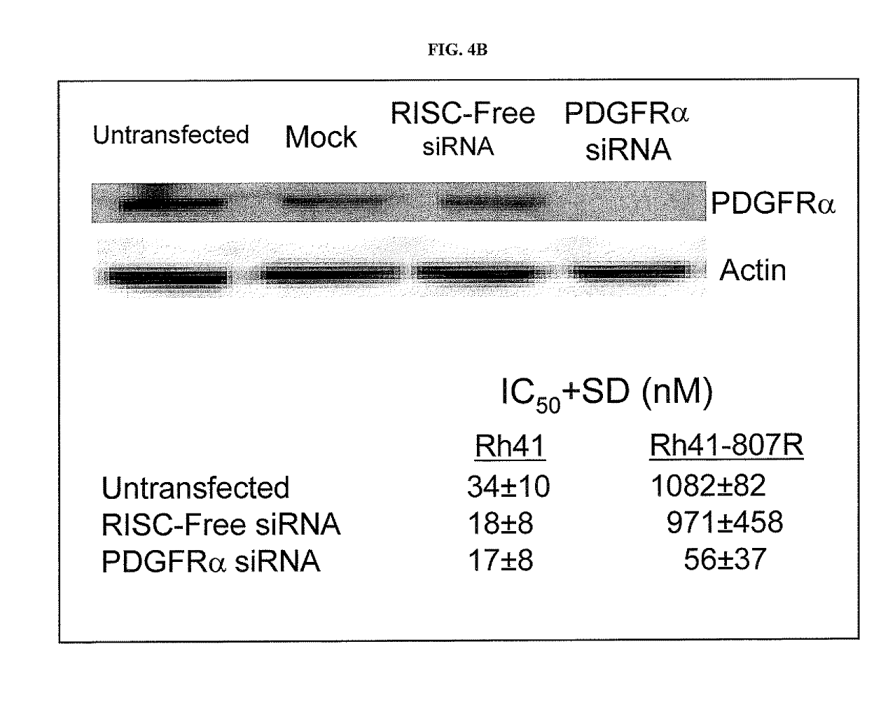

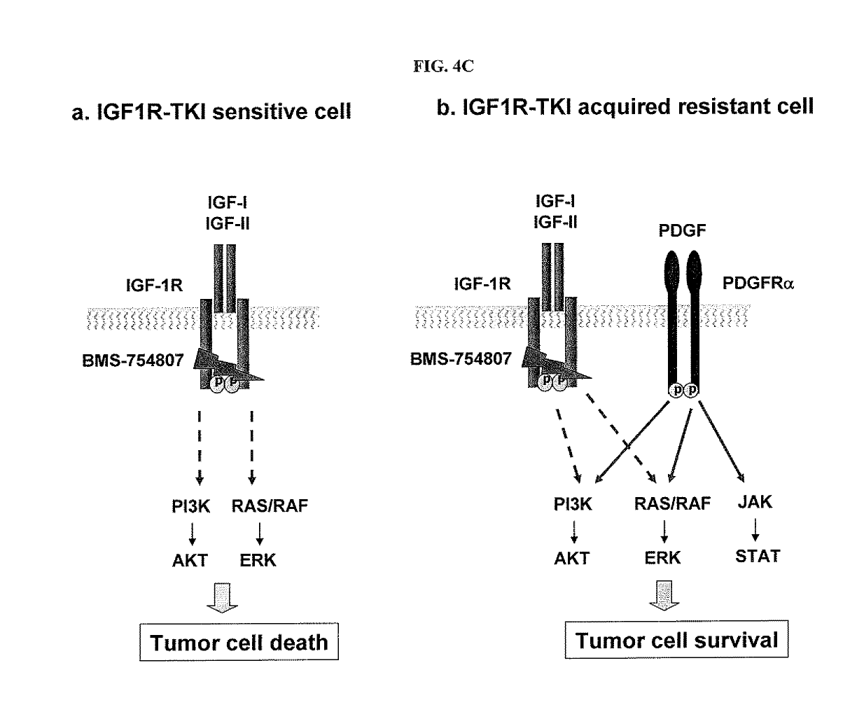

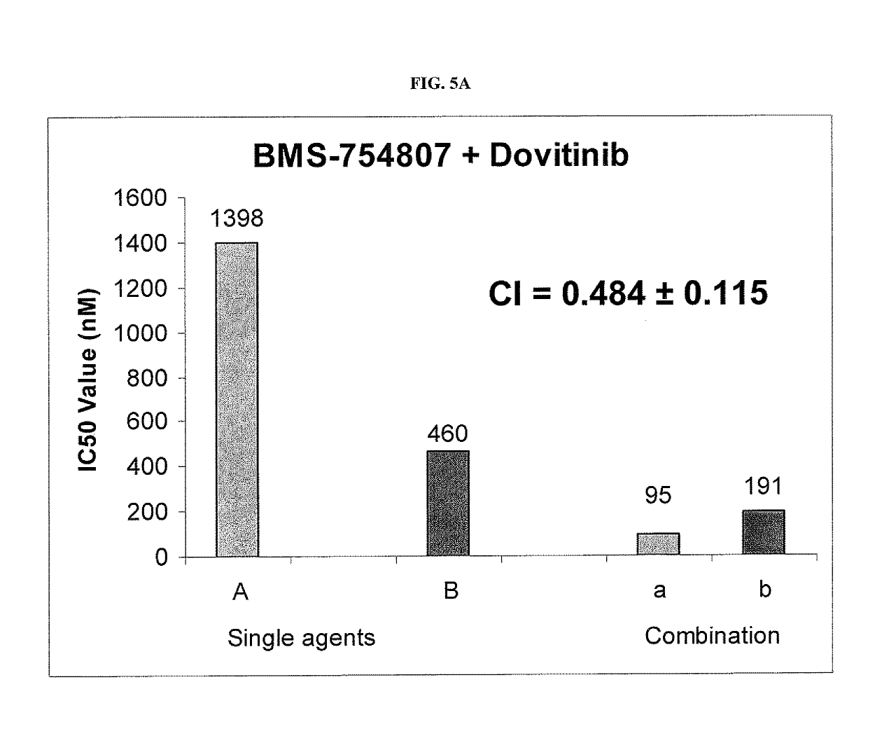

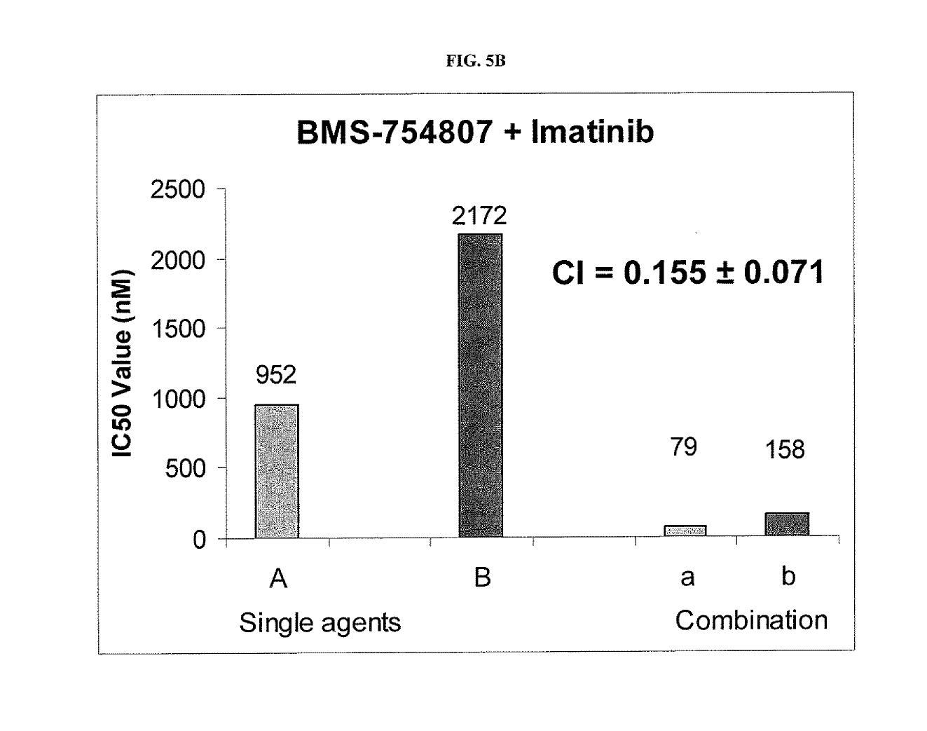

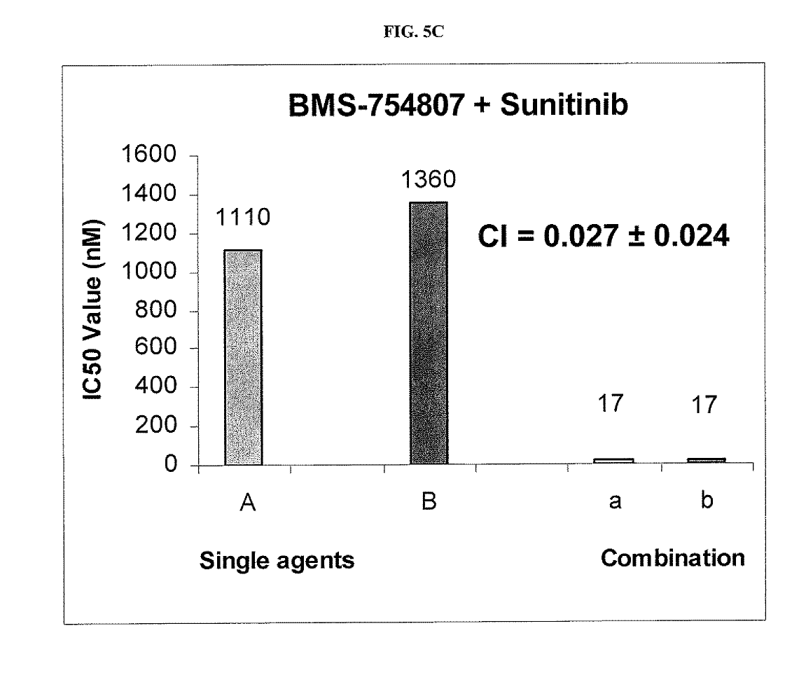

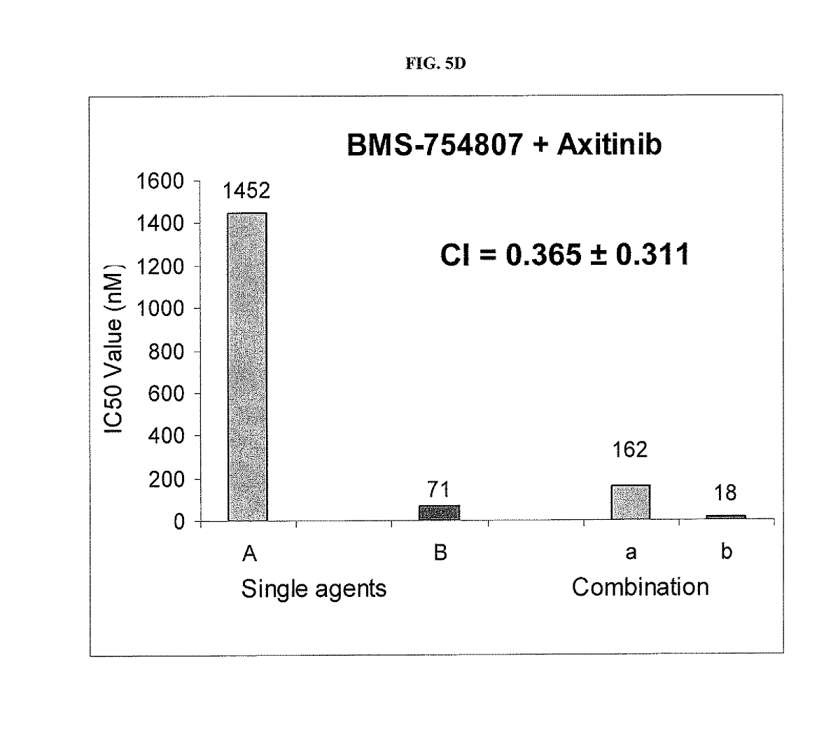

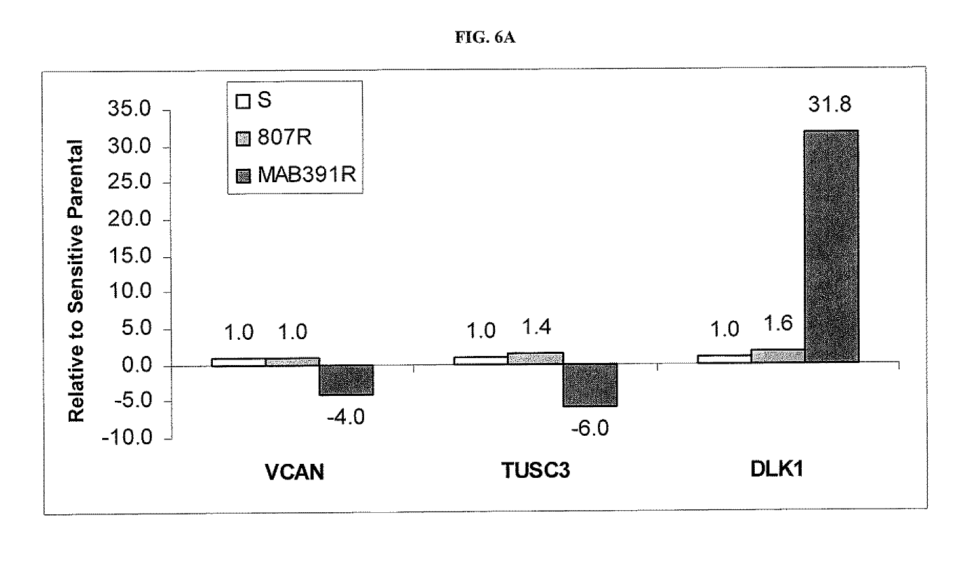

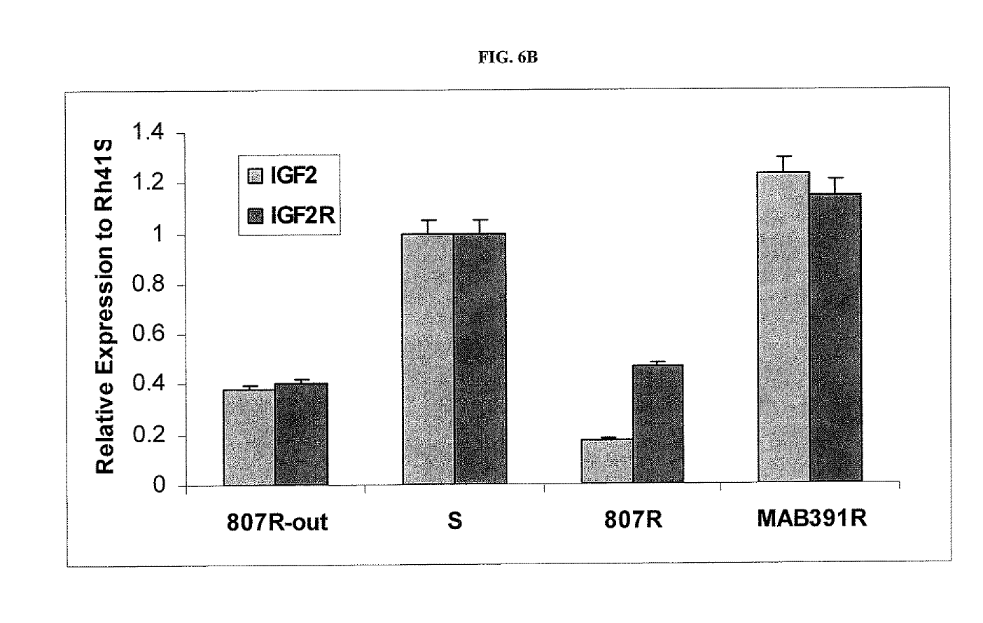

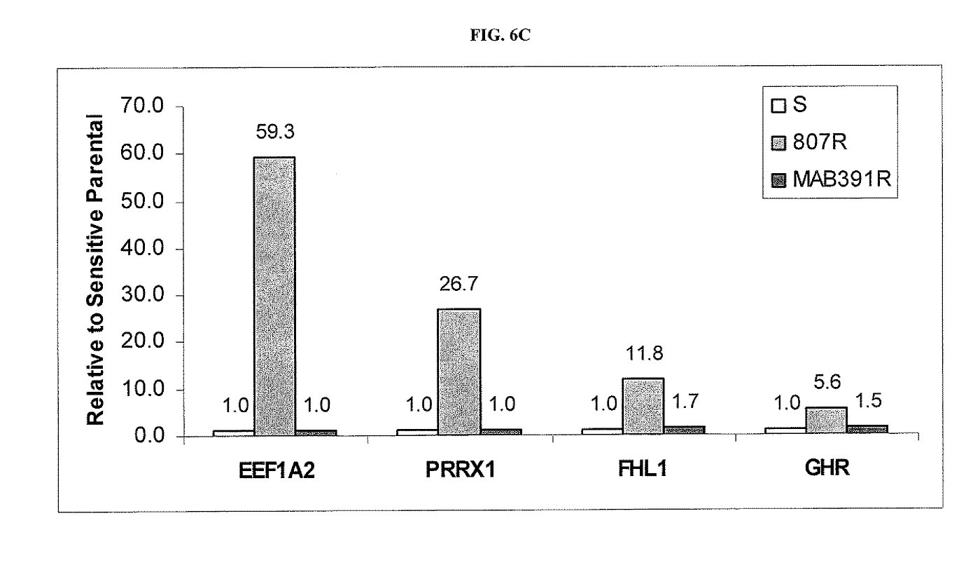

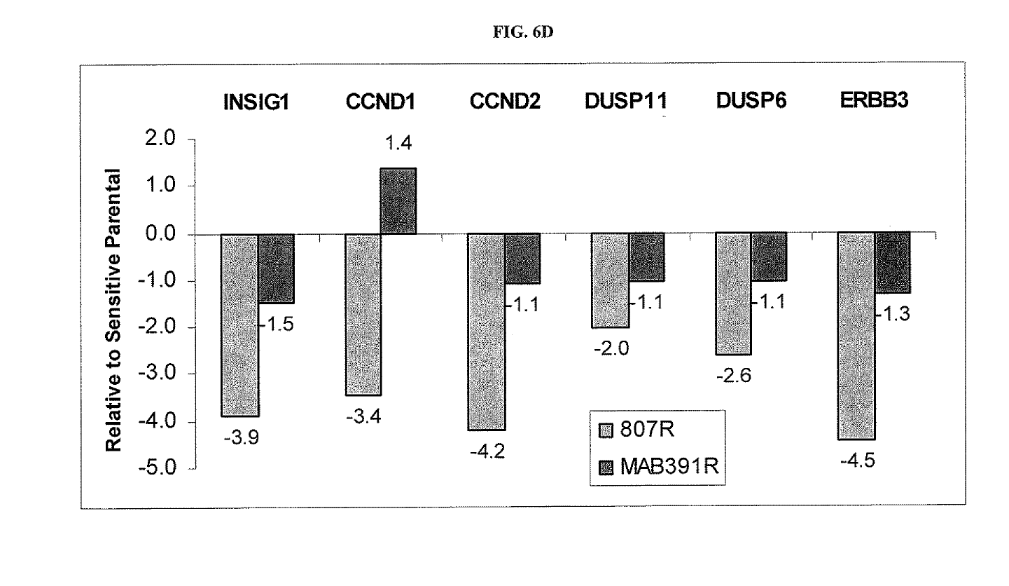

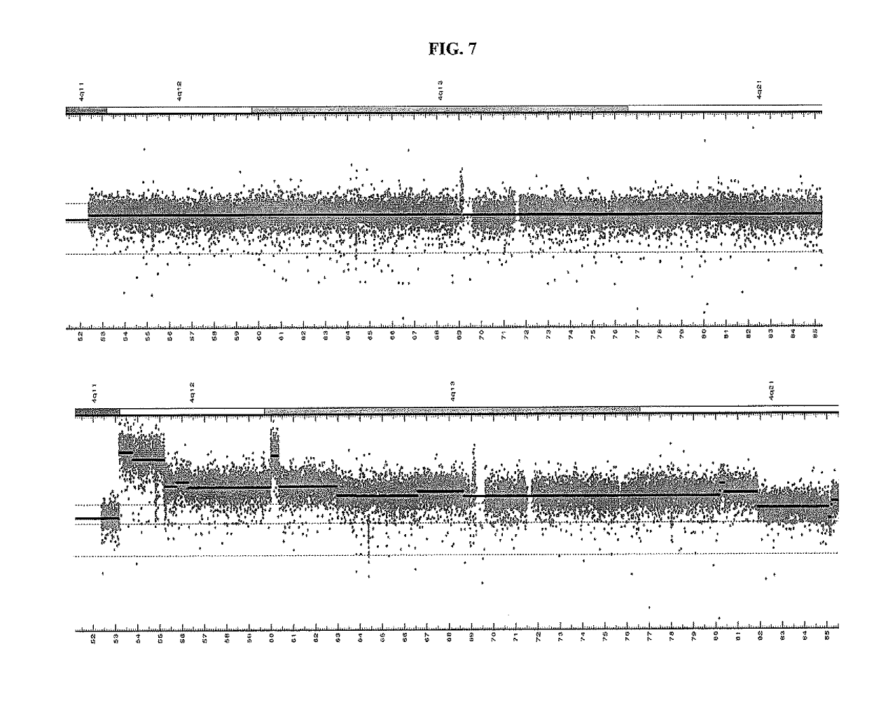

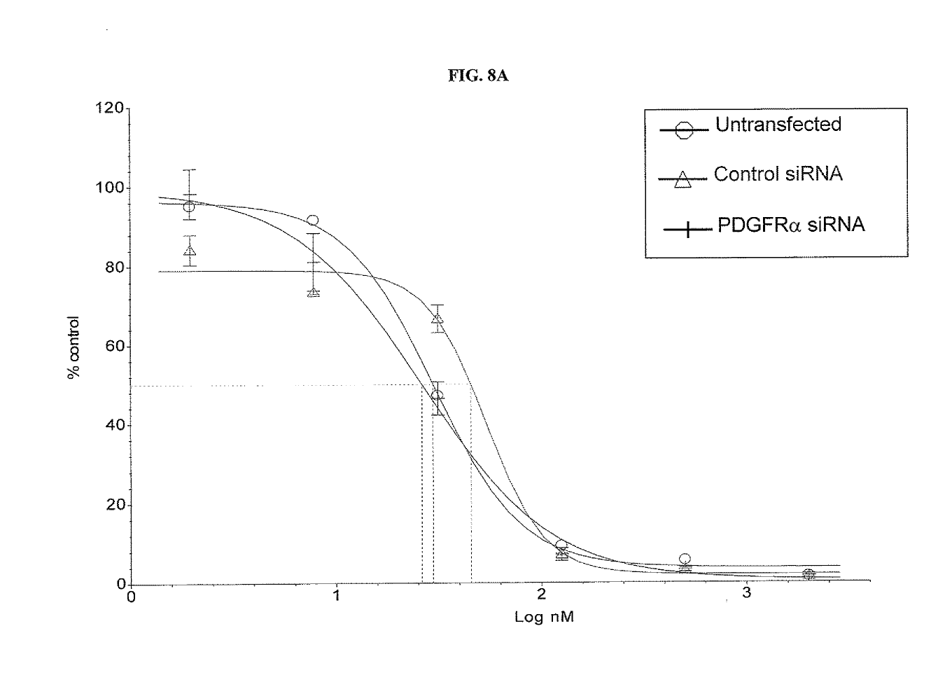

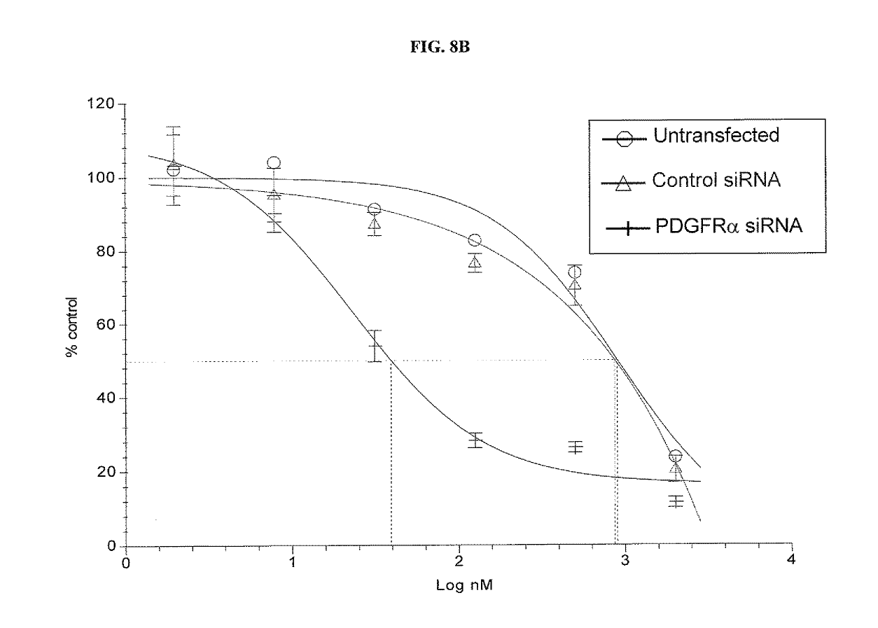

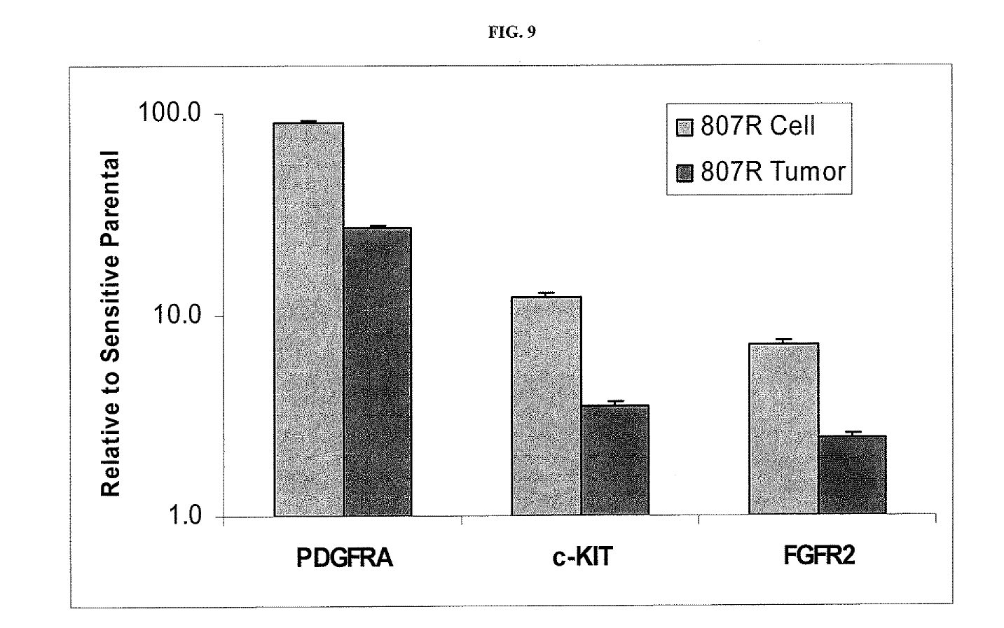

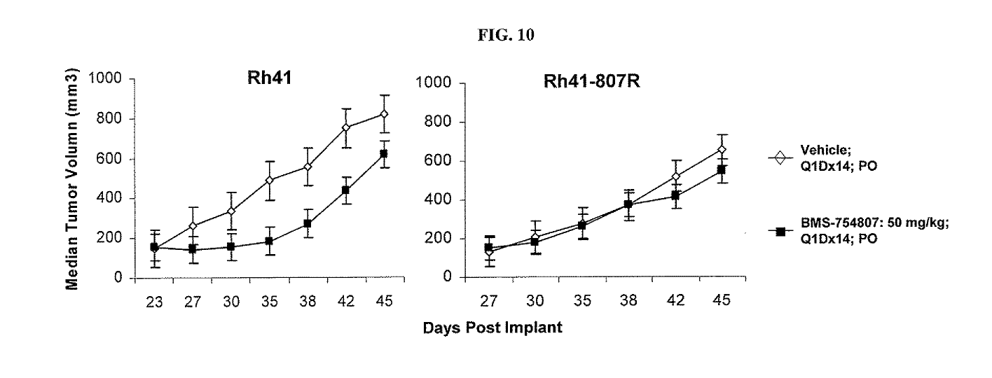

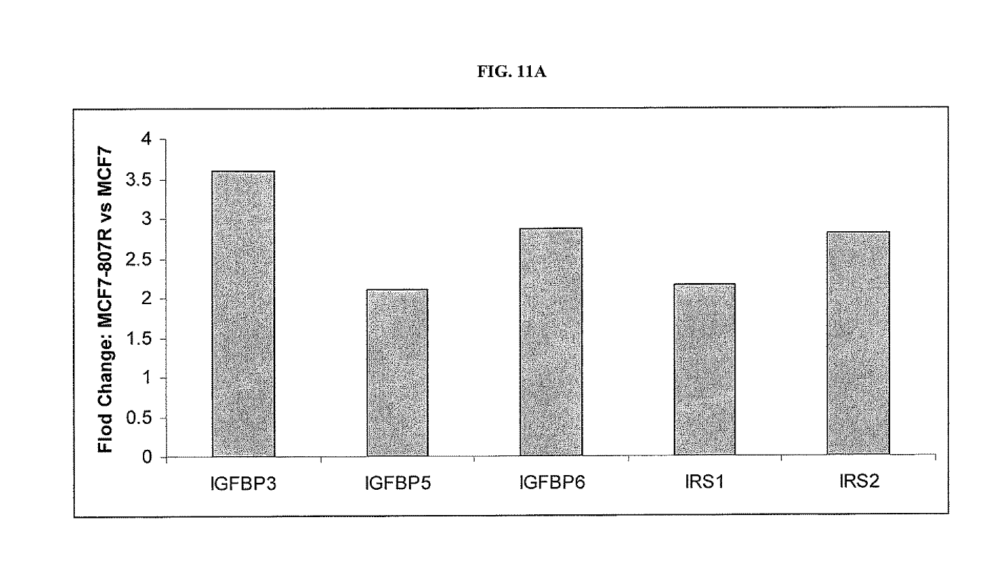

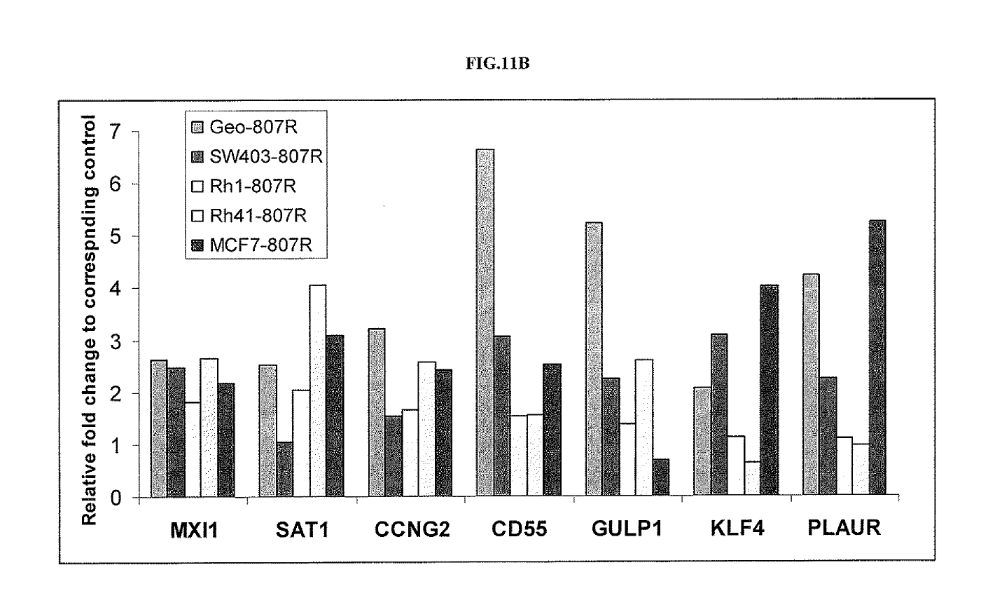

Genetic alterations that lead to overexpression or altered function of the gene product in tumors have been implicated in drug resistance (Jänne et al., Nat. Rev. Drug Discov., 8(9):709-723 (2009)). One of the possible resistance mechanisms could be, for instance, cells that acquire specific gene disregulation which may then result in overexpression of some signaling molecules. Indeed, the present inventors found DNA copy number gains in 807R cells in chr4q12, where PDGFR-α and c-KIT are located (FIG. 2D). Overexpression of PDGFR-α appears to precede amplification of the gene. 807R cells had significantly increased expression of PDGFR-α and c-KIT (FIGS. 3A and 3C). Induction of PDGFRα RNA in Rh41 cells was an early event and seen at 4 hrs after BMS-754807 treatment (FIGS. 3F and 3G), and this induction reached >89 fold increase when Rh41 continued exposure to BMS-754807 until became to Rh41-807R (FIG. 3A).

Our data provide strong evidence that PDGFR-α plays a direct role: increased expression of PDGFR-α in BMS-754807 acquired-resistant cell 807R; PDGFR-a-targeted siRNA restores BMS-754807 sensitivity in 807R cells. These results indicate PDGFR-α confers a mechanism of acquired resistance to BMS-754807 in this model system. PDGFR-α amplification has been reported especially in glioblastomas and sarcoma (Fleming et al., Cancer Res., 52:4550-4553 (1992); Zhao et al., Genes Chromosomes Cancer, 34:48-57 (2002)), but its role involved in drug resistance has not been identified previously. This study is the first one to link the amplification/overexpression of PDGFR-α to the mechanism of resistance to IGF-1R inhibitor.

KIT or PDGFR-α activating mutations are the pathogenic mechanisms that characterize gastrointestinal stromal tumors (GIST). Different mechanisms of acquired resistance to tyrosine kinase inhibitors have been linked to the acquisition of new molecular abnormalities associated with KIT and PDGFRA receptor signaling pathway in GISTs including loss of KIT expression; the genomic amplification of KIT; the activation of an alternative downstream signaling pathways such as AKT/mTOR; and the acquisition of new receptor mutations (Maleddu et al., Oncol. Rep., 21(6):1359-1366 (June 2009). It has been recently reported that in chronic eosinophilic leukemia (a disease characterized by the FIP1L1-PDGFR-α fusion gene), acquired resistance to imatinib was mediated by a T6741 mutation in the ATP-binding pocket of PDGFR-α, and D842V mutant of FIP1L1-PDGFR-α has emerged during treatment with sorafenib after short clinical response (Lierman et al., Leukemia, 23(5):845-851 (May 2009)). So it is possible that another resistance mechanism to the small molecule IGF-1R inhibitor BMS-754807 could be cells that acquire specific activating mutations on genes such as PDGFR-α or c-KIT. This will require further investigation.

The cross-talk between IGF-1R and PDGFR pathways to enhance tumor cells proliferation and induce resistance to therapy are not fully understood. There are overlaps on a common set of signaling nodes that are part of downstream effectors of IGF-1R or PDGFR-α signaling. These are PI3K/AKT and RAS/RAF/MEK/ERK signaling molecules which are integrators of growth and survival signals originating from IGF-1R or PDGFR-α. The present inventors hypothesize (FIG. 4C) that in the sensitive Rh41 cells, IGF-1R is the predominant driver for survival via activation of AKT and MAPK, which can be inhibited by BMS-754807, thus sensitive to the drug; whereas in 807R cells, acquisition of alternate receptor tyrosine kinase PDGFR-α through gene amplification leading to its overexpression and activation, hence increasing proliferation and survival signals through these two shared and cross-talked pathways. In this case, targeting IGF-1R is not enough and inactivation of common proteins that are key transmitters in two signaling pathways of IGF-1R and PDGFR-α is of central importance to interrupt their survival signaling. This will undoubtedly require combination of targeting these two redundant signaling pathways to result enhanced inhibition of tumor cell growth (FIG. 5). With the realization of acquired resistance to targeted agents emerging, evolving strategies including sequential treatment, concurrent treatment, and biomarker development should be further considered when developing targeted agents.

Recent studies report that increased expression of AXL, a membrane-bound receptor tyrosine kinase, may confer acquired resistance to imatinib in gastrointestinal tumors (Mahadevan et al., Oncogene, 26:3909-3919 (2007)), lapatinib in breast tumor cells (Liu et al., Cancer Res., 69(17):6871-6878 (2009)) and chemotherapy drugs in CML (Hong et al., Cancer Lett., 268:314-324 (2008)). The present inventors observed overexpression of AXL in MAB391R cells but not in 807R cells, it is not clear the reason for the difference. The role of AXL in resistance to anti-IGF1R antibody needs to be further explored.

The differences between small molecules and antibodies have been addressed by comparing erlotinib and cetuximab for targeting EGFR or lapatinib and trastuzumab for targeting HER2 (Imai et al., Nat. Rev. Cancer, 6:714-727 (2006)). One of the most important differences is the disparity in selectivity. It is difficult to predict differences in efficacy between anti-receptor antibodies and small-molecule inhibitors (Mendelsohn et al., Semin. Oncol., 33:369-385 (2006)). The differences between tyrosine kinase inhibitors versus antibodies against IGF-IR resemble those between the mentioned agents. Due to the difference, both types of agents may also have different mechanisms for acquired resistance. Indeed, from our study, apparently the mechanisms leading to resistance to the small molecule IGF-1R inhibitor BMS-754807 differ from mechanisms leading to resistance to IGF-1R antibody MAB391. Furthermore, cells that developed resistance to BMS-754807 are cross-resistant to other IGF-1R inhibitors including anti-IGF-1R antibody MAB391, but cell line resistant to MAB391 is still reasonably sensitive to BMS-754807 (Table 1). Thus, one may hypothesize that patients who fail treatment of IGF-1R antibody therapies may still benefit from BMS-754807 treatment due to its wider spectrum against IGF-IR, IR and hybrid receptors thus might be more effective than the antibodies. BMS-754807 is currently in clinical development for the treatment of a variety of human cancers, and several testable hypotheses could be evaluated in these clinical studies.

Additional studies were performed to identify other genes and signaling pathways that may provide a resistant mechanism to IGF-1R inhibitors. Specifically, additional four cell lines of different tumor types, MCF7, Rh41, Rh1, Geo and SW480 (breast, sarcomas and colon), were induced to develop acquired resistance to BMS-754807 by stepwise exposure to increasing concentrations of the drug for extended periods. Analyses of in vitro drug response and gene expression profiles were performed to characterize the resistant models and the corresponding sensitive parental cells. Cell line specific as well as shared molecular alterations were observed in the different resistant cells using genomic approaches to define mechanisms of resistance to BMS-754807. The resistant models were also tested against multiple IGF-1R inhibitors and showed cross-resistance suggesting common mechanisms of resistance to IGF-1R inhibition.

MCF-807R cells showed increases in IGFBP3, IGFBP5 and IGFBP6 RNA expression levels compared to the sensitive parental cells.

In summary, the present inventors identified and compared the acquired resistance mechanisms between small molecular inhibitor BMS-754807 and MAB391, an antibody against IGF-IR. Acquired resistance to BMS-754807 was associated with increased expression of PDGFR-α and c-KIT. Crosstalk between IGF-1R and PDGFR-α can confer acquired resistance to IGF-1R inhibition through compensatory mechanisms by the enhanced activity of PDGFR pathway. Dual PDGFR and IGFR inhibition may prevent or reverse resistance to IGFR inhibitors offering a promising strategy for exploration in clinical studies to yield greater anticancer activity. These molecular changes could serve as biomarkers in identifying resistant tumors in clinical trials. By elucidating molecular mechanisms of acquired resistance to IGF-1R inhibitors in this study, it provides the basis for rationally developing next generation inhibitors as well as effective drug combinations that can overcome or prevent acquired resistance to the inhibitors, thereby enhancing clinical benefit.

### Biomarkers and Biomarker Sets

The invention includes individual biomarkers and biomarker sets having both diagnostic and prognostic value in proliferative disease areas in which IGF-1R is of importance, e.g., in cancers or tumors, or in disease states in which cell signaling and/or cellular proliferation controls are abnormal or aberrant. The biomarker sets comprise a plurality of biomarkers that highly correlate with resistance or sensitivity to one or more IGF-1R agents.

The biomarkers and biomarker sets of the invention enable one to predict or reasonably foretell the likely effect of one or more IGF-1R agents in different biological systems or for cellular responses merely based upon whether one or more of the biomarkers of the present invention are overexpressed relative to normal. The biomarkers and biomarker sets can be used in in vitro assays of cellular proliferation by sample cells to predict in vivo outcome. In accordance with the invention, the various biomarkers and biomarker sets described herein, or the combination of these biomarker sets with other biomarkers or markers, can be used, for example, to predict and monitor how patients with cancer might respond to therapeutic intervention with one or more IGF-1R inhibitors.

Measuring the level of expression of a biomarker and biomarker set provides a useful tool for screening one or more tumor samples before treatment of a patient with the microtubulin-stabilizing agents. The screening allows a prediction of whether the cells of a tumor sample will respond favorably to the microtubulin-stabilizing agents, based on the presence or absence of over-expression—such a prediction provides a reasoned assessment as to whether or not the tumor, and hence a patient harboring the tumor, will or will not respond to treatment with the microtubulin-stabilizing agents.

A difference in the level of the biomarker that is sufficient to indicate whether the mammal will or will not respond therapeutically to the method of treating cancer can be readily determined by one of skill in the art using known techniques. The increase or decrease in the level of the biomarker can be correlated to determine whether the difference is sufficient to identify a mammal that will respond therapeutically. The difference in the level of the biomarker that is sufficient can, in one aspect, be predetermined prior to determining whether the mammal will respond therapeutically to the treatment. In one aspect, the difference in the level of the biomarker is a difference in the mRNA level (measured, for example, by RT-PCR or a microarray), such as at least about a two-fold difference, at least about a three-fold difference, or at least about a four-fold difference in the level of expression, or more. In another aspect, the difference in the level of the biomarker is determined at the protein level by mass spectral methods or by FISH or by IHC. In another aspect, the difference in the level of the biomarker refers to a p-value of <0.05 in Anova analysis. In yet another aspect, the difference is determined in an ELISA assay.

The biomarker or biomarker set(s) outlined herein can also be used as described herein for monitoring the progress of disease treatment or therapy in those patients undergoing treatment for a disease involving an IGF1R inhibitor treatment.

The biomarkers also serve as targets for the development of therapies for disease treatment. Such targets may be particularly applicable to treatment of cancer, such as, for example, breast and/or lung cancer.

Indeed, because these biomarkers are differentially expressed in sensitive and resistant cells, their expression patterns are correlated with relative intrinsic sensitivity of cells to treatment with IGF-1R inhibitors. Accordingly, the biomarkers over expressed in resistant cells may serve as targets for the development of new therapies for the tumors which are resistant to IGF-1R inhibitors. The level of biomarker protein and/or mRNA can be determined using methods well known to those skilled in the art. For example, quantification of protein can be carried out using methods such as ELISA, 2-dimensional SDS PAGE, Western blot, immunoprecipitation, immunohistochemistry, fluorescence activated cell sorting (FACS), or flow cytometry. Quantification of mRNA can be carried out using methods such as PCR, array hybridization, Northern blot, in-situ hybridization, dot-blot, TAQMAN®, or RNAse protection assay.

The present invention encompasses the use of any one or more of the following as a biomarker for use in predicting IGF-1R inhibitors response: IGF-1R, PDGFR-α, AXL, and EGFR.

The present invention also encompasses any combination of the aforementioned biomarkers, including, but not limited to: IGF-1R, PDGFR-α, and AXL; IGF-1R, PDGFR-α, and AXL; IGF-1R and PDGFR-α; PDGFR-α and AXL; AXL; IGF-1R; IGF-1R and AXL; IGF-1R, PDGFR-α; and PDGFR-α; and PDGFR-α, AXL; or each marker individually.

Identification of biomarkers that provide rapid and accessible readouts of efficacy, drug exposure, or clinical response is increasingly important in the clinical development of drug candidates. Embodiments of the invention include measuring changes in the levels of mRNA and/or protein in a sample to determine whether said sample contains increased or decreased expression of IGF-1R, PDGFR-α, AXL. In one aspect, said samples serve as surrogate tissue for biomarker analysis. These biomarkers can be employed for predicting and monitoring response to one or more microtubulin-stabilizing agents. In one aspect, the biomarkers of the invention are one or more of the following: IGF-1R, PDGFR-α, AXL, including both polynucleotide and polypeptide sequences. In another aspect, the biomarkers of the invention are nucleotide sequences that, due to the degeneracy of the genetic code, encodes for a polypeptide sequence provided in the sequence listing.

The biomarkers serve as useful molecular tools for predicting and monitoring response to IGF-1R inhibitors.

Methods of measuring the level of any given marker described herein may be performed using methods well known in the art, which include, but are not limited to PCR; RT-PCR; FISH; IHC; immunodetection methods; immunoprecipitation; Western Blots; ELISA; radioimmunoassays; FACS; HPLC; surface plasmon resonance, and optical spectroscopy; and mass spectrometry, among others.

The biomarkers of the invention may be quantified using any immunospecific binding method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., Current Protocols in Molecular Biology, Vol. 1, John Wiley & Sons, Inc., New York (1994), which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% TRASYLOL®) supplemented with protein phosphatase and/or protease inhibitors (e.g., EDTA, PMSF, aprotinin, sodium vanadate), adding the antibody of interest (i.e., one directed to a biomarker of the present invention) to the cell lysate, incubating for a period of time (e.g., 1-4 hours) at 4° C., adding protein A and/or protein G SEPHAROSE® beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with SEPHAROSE® beads). For further discussion regarding immunoprecipitation protocols see, e.g., Ausubel et al., eds., Current Protocols in Molecular Biology, Vol. 1, p. 10.16.1, John Wiley & Sons, Inc., New York (1994).

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds., Current Protocols in Molecular Biology, Vol. 1, p. 10.8.1, John Wiley & Sons, Inc., New York (1994).

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds., Current Protocols in Molecular Biology, Vol. 1, p. 11.2.1, John Wiley & Sons, Inc., New York (1994).

Alternatively, identifying the relative quantitation of the biomarker polypeptide(s) may be performed using tandem mass spectrometry; or single or multi dimensional high performance liquid chromatography coupled to tandem mass spectrometry. The method takes into account the fact that an increased number of fragments of an identified protein isolated using single or multi dimensional high performance liquid chromatography coupled to tandem mass spectrometry directly correlates with the level of the protein present in the sample. Such methods are well known to those skilled in the art and described in numerous publications, for example, Link, A. J., ed., 2-D Proteome Analysis Protocols, Humana Press (1999), ISBN: 0896035247; Chapman, J. R., ed., Mass Spectrometry of Proteins and Peptides, Humana Press (2000), ISBN: 089603609X.

As used herein the terms “modulate” or “modulates” or “modulators” refer to an increase or decrease in the amount, quality or effect of a particular activity, or the level of DNA, RNA, or protein detected in a sample.

In order to facilitate a further understanding of the invention, the following examples are presented primarily for the purpose of illustrating more specific details thereof. The scope of the invention should not be deemed limited by the examples, but to encompass the entire subject matter defined by the claims.

### BRIEF DESCRIPTION OF THE SEQUENCE LISTING

Incorporated herein by reference in its entirety is a Sequence Listing, comprising SEQ ID NO:1 through SEQ ID NO:36, which include nucleic acid and amino acid sequences of the biomarkers presented herein. The Sequence Listing is contained on a compact disc, i.e., CD-ROM, three identical copies of which are filed herewith. The Sequence Listing, in IBM/PC MS-DOS (ASCII) text format, was first created on Oct. 29, 2010, and is 144 KB in size.

### REFERENCES

1. Jänne, P. A. et al., “Factors underlying sensitivity of cancers to kinase inhibitors”, Nat. Rev. Drug Discov., 8(9):709-723 (2009).
2. Engelman, J. A. et al., “Acquired resistance to tyrosine kinase inhibitors during cancer therapy”, Curr. Opin. Genet. Dev., 18:73-79 (2008).
3. Gone, M. E. et al., “Clinical resistance to STI-571cancer therapy caused by BCR-ABL gene mutation or amplification”, Science, 293(5531):876-880 (2001).
4. Donato, N. J. et al., “BCR-ABL independence and LYN kinase overexpression in chronic myelogenous leukemia cells selected for resistance to STI571”, Blood, 101 (2): 690-698 (2003).
5. Mahadevan, D. et al., “A novel tyrosine kinase switch is a mechanism of imatinib resistance in gastrointestinal tumors”, Oncogene, 26:3909-3919 (2007).
6. Illmer, T. et al., “P-glycoprotein-mediated drug efflux is a resistance mechanism of chronic myelogenous leukemia cells to treatment with imatinib mesylate”, Leukemia, 18(3):401-408 (2004).
7. Samani, A. A. et al., “The role of the IGF system in cancer growth and metastasis:overview and recent insights”, Endocr. Rev., 28:20-47 (2007).
8. Denley, A. et al., “Molecular interactions of the IGF system”, Cytokine Growth Factor Rev., 16:421-439 (2005).
9. Pandini, G. et al., “Insulin and insulin-like growth factor-I (IGFI) receptor overexpression in breast cancers leads to insulin/IGF-I hybrid receptor overexpression: evidence for a second mechanism of IGF-I signaling”, Clin. Cancer Res., 5:1935-1944 (1999).
10. Morrione, A. et al., “Insulin-like growth factor II stimulates cell proliferation through the insulin receptor”, Proc. Natl. Acad. Sci. USA, 94:3777-3782 (1997).
11. Law, J. H. et al., “Phosphorylated insulin-like growth factor-i/insulin receptor is present in all breast cancer subtypes and is related to poor survival”, Cancer Res., 68(24):10238-10246 (Dec. 15, 2008).
12. Gualberto, A. et al., “Emerging role of insulin-like growth factor receptor inhibitors in oncology: early clinical trial results and future directions”, Oncogene, 28(34):3009-3021 (2009).
13. Rodon, J. et al., “Early drug development of inhibitors of the insulin-like growth factor-I receptor pathway: Lessons from the first clinical trials”, Mol. Cancer. Ther., 7(9):2575-2588 (2008).
14. Weroha, S. J. et al., “IGF-1 receptor inhibitors in clinical trials—Early lessons”, J. Mamm. Gland Biol. Neoplasia, 13:471-483 (2008).
15. Carboni, J. M. et al., “BMS-754807, a small molecule inhibitor of IGF1R for clinical development”, Proceedings of the 100th Annual Meeting of the American Association for Cancer Research, 2009 Apr. 18-22; Denver, Colo., Abstract No. 1742.
16. Clements et al., AACR-NCI-EORTC Molecular Targets and Cancer Therapeutics Meeting 2009, Abstract No. A101.
17. Desai et al., AACR-NCI-EORTC Molecular Targets and Cancer Therapeutics Meeting 2009, Abstract No. A109.
18. Olmos, D. et al., “Safety, pharmacokinetics and preliminary activity of the anti-IGF-IR antibody CP-751,871 in patients with sarcoma”, J. Clin. Oncol., 26:553s (2008).
19. Tolcher, A. W. et al., “A phase I pharmacokinetic and pharmacodynamic study of AMG 479, a fully human monoclonal antibody against insulin-like growth factor type 1 receptor (IGF-1R), in advanced solid tumors”, J. Clin. Oncol., 25:118s (2007).
20. Karp, D. D. et al., “High activity of the anti-IGF-IR antibody CP-751,871 in combination with paclitaxel and carboplatin in squamous NSCLC”, ASCO Meeting Abstracts 2008, 26 15_supp1:8015.
21. Haluska, P. et al., “Phase I dose escalation study of the anti-IGF-1R monoclonal antibody CP-751,871 in patients with refractory solid tumors”, ASCO Meeting Abstracts 2007, 25 18_supp1:3586.
22. Hailey, J. et al., “Neutralizing anti-insulin-like growth factor receptor 1 antibodies inhibit receptor function and induce receptor degradation in tumor cells”, Mol. Cancer. Ther., 1:1349-1353 (2002).
23. Bengtsson, H. et al., “A single-array preprocessing method for estimating full-resolution raw copy numbers from all Affymetrix genotyping arrays including GenomeWideSNP 5 & 6”, Bioinformatics, 25(17):2149-2156 (2009).
24. Olshen, A. B. et al., “Circular binary segmentation for the analysis of array-based DNA copy number data”, Biostatistics, 5:557-572 (2004).
25. Huang, F. et al., “The mechanisms of differential sensitivity to an insulin-like growth factor-1 receptor inhibitor (BMS-536924) and rationale for combining with EGFR/HER2 inhibitors”, Cancer Res., 69(1):161-170 (Jan. 1, 2009).
26. Homsi, J. et al., “Spectrum of activity and mechanism of action of VEGF/PDGF inhibitors”, Cancer Control, 14:285-294 (2007).
27. Zha, J. et al., “Molecular predictors of response to a humanized anti-insulin-like growth factor-I receptor monoclonal antibody in breast and colorectal cancer”, Mol. Cancer. Ther., 8(8):2110-2121 (August 2009).
28. Gottesman, M. M. et al., “Multidrug resistance in cancer: Role of ATP-dependent transporters”, Nat. Rev. Cancer, 2:48-58 (2002).
29. Gooch, J. L. et al., “Insulin-like growth factor (IGF)-I rescues breast cancer cells from chemotherapy-induced cell death-proliferative and antiapoptotic effects”, Breast Cancer Res. Treat., 56:1-10 (1999).
30. Fleming, T. P. et al., “Amplification and/or overexpression of platelet-derived growth factor receptors and epidermal growth factor receptor in human glial tumors”, Cancer Res., 52:4550-4553 (1992).
31. Zhao, J. et al., “Combined comparative genomic hybridization and genomic microarray for detection of gene amplifications in pulmonary artery intimal sarcomas and adrenocortical tumors”, Genes Chromosomes Cancer, 34:48-57 (2002).
32. Maleddu, A. et al., “Mechanisms of secondary resistance to tyrosine kinase inhibitors in gastrointestinal stromal tumours (Review)”, Oncol. Rep., 21(6):1359-1366 (June 2009).
33. Lierman, E. et al., “FIP1L1-PDGFRalpha D842V, a novel panresistant mutant, emerging after treatment of FIP1L1-PDGFRalpha T674I eosinophilic leukemia with single agent sorafenib”, Leukemia, 23(5):845-851 (May 2009).
34. Liu, L. et al., “Novel Mechanism of Lapatinib Resistance in HER2-Positive Breast Tumor Cells: Activation of AXL”, Cancer Res., 69(17):6871-6878 (2009).
35. Hong, C. C. et al., “Receptor tyrosine kinase AXL is induced by chemotherapy drugs and overexpression of AXL confers drug resistance in acute myeloid leukemia”, Cancer Lett., 268:314-324 (2008).
36. Imai, K. et al., “Comparing antibody and small-molecule therapies for cancer”, Nat. Rev. Cancer, 6:714-727 (2006).
37. Mendelsohn, J., “Epidermal growth factor receptor targeting in cancer”, Semin. Oncol., 33:369-385 (2006).
38. Hou, X. et al., “Combined estrogen signaling inhibition and IGF-1R/InsR inhibition with BMS-754807 in estrogen-dependent breast cancer model has synergistic antitumor activity in vitro and in vivo”, 100th Annual Meeting of the American Association for Cancer Research, Abstract No. 1743 (Apr. 19, 2009).
39. Wittman, M. D. et al., “Discovery of BMS-754807, a small molecule inhibitor of IGF-1R in clinical development”, 237th National Meeting of the American Chemical Society, MEDI Abstract No. 163 (Mar. 24, 2009).
40. Litzenburger, B. C. et al., “BMS-536924 Reverses IGF-1R-Induced Transformation of Mammary Epithelial Cells and Causes Growth Inhibition and Polarization of MCF7 Cells”, Clin. Cancer Res., 15(1):226-237 (2009).
41. Jänne P. A. et al., “Factors underlying sensitivity of cancers to kinase inhibitors”, Nat. Rev. Drug Discov., 8:709-723 (2009).
42. Hendrickson, A. W. et al., “Resistance pathways relevant to insulin-like growth factor-1 receptor-targeted therapy”, Curr. Opin. Investig. Drugs, 10:1032-1040 (2009).

### EXAMPLES

### Materials and Methods

### Establishment of Resistant Cell Lines to IGF-1R Inhibitors

Human rhabdomyosarcoma cell line Rh41 was provided by Dr. Lee Helman and grown in RPMI medium plus GLUTAMAX® supplemented with 10% fetal bovine serum (FBS), 10 mmol/L HEPES, penicillin, and streptomycin. To develop acquired resistant cells to either BMS-754807, or mAB391 (R&D Systems, Inc., Minneapolis, Minn.), the sensitive Rh41 cells were exposed to the corresponding drug at the IC₅₀ concentration and then at gradually increasing concentrations every other culture passage. The IC₅₀ value to the compound was measured periodically during this treatment time until the resistance level reached a plateau.

### In Vitro Cellular Proliferation Assays

Cell proliferation was evaluated by [³H]-thymidine incorporation after exposure to either BMS-754807 or mAB391 for 72 hours. Cells were plated at an optimized density in 96-well plates, incubated overnight at 37° C., and then exposed to a serial dilution of the drugs. After 72 hours incubation, cells were pulsed with 4 μCi/ml [³H]-thymidine (Amersham Pharmacia Biotech, UK) for 3 hours, trypsinized, harvested onto UNIFILTER®-96 GF/B plates (PerkinElmer, Boston, Mass.); scintillation was measured on a TOPCOUNTO NXT (Packard, Conn.). Results were expressed as an IC₅₀, which is the drug concentration required to inhibit cell proliferation by 50% compared to untreated control cells. The mean IC₅₀ and standard deviation from multiple tests for each cell line were calculated.

### Gene Expression Profiling

RNA was isolated from both parental and acquired resistant cells using the RNEASY® kits from Qiagen (Valencia, Calif.) for generating gene expression data using Affymetrix HT-HG-U133A GENECHIP® (Affymetrix, Santa Clara, Calif.) according to the manufacture manual. Microarray data were analyzed and visualized using PARTEK® and Cluster/Treeview software. Genes differentially expressed between resistant cell lines and sensitive parental were identified using t-test.

### Gene Copy Analysis

DNA was isolated from 5×10⁶ cells using the DNeasy Blood and Tissue kit from Qiagen (Valencia, Calif.). Two aliquots of 250 ng genomic DNA per sample were digested by restriction enzymes NspI and Styl, respectively. The resulted products were ligated to the corresponding adaptors and PCR amplified. The labeled PCR products were hybridized to the Human SNP 6.0 array according to the Affymetrix recommendations. The Cel files were processed using aroma.affymetrix package (Bengtsson et al., “GenomeWideSNP 5 & 6”, Bioinformatics, 25(17):2149-2156 (2009)) in the R-project. Segmentation of normalized raw copy number data was performed with the CBS algorithm (Olshen et al., Biostatistics, 5:557-572) implemented in the aroma.affymetrix package. Copy number gain (or loss) of a gene was obtained by taking the maximum (or minimum) of segmented copy number values within the genomic region of the gene.

### Western Blot Analyses

Cells were treated as indicated in the figure legends. Protein extraction, quantization, western blots and protein visualization are described previously (Huang et al., Cancer Res., 69(1):161-170 (Jan. 1, 2009)).

### Flow Cytometry for IGF-1R Expression

Rh41-MAB391R cells were seeded in six-well plates in duplicate in presence of 3 nM mAB391 (R&D Systems, Minneapolis, Minn.) and cultured overnight. The medium was removed and fresh medium without the mAB391 was added. Cells were collected at 0.5, 1, 2, 4, 16, and 24 hours post washout, stained with IGF-1R antibody (BD, Franklin Lakes, N.J.) and analyzed by FACscan (FACS Calibur, Becton Dickson).

Small Interfering RNA (siRNA)

Rh41 and Rh41-807R cells transfections were carried out using ON-TARGETplus siRNA to human PDGFRA (Dharmacon, Chicago, Ill.) with DharmaFECT transfection reagents and Opti-MEM media (Invitrogen, Carlsbad, Calif.) according to DharmaFECT General Transfection Protocol. RISC-free siRNA was transfected as the negative control (Dharmacon). Following the addition of the drugs, cells were incubated at 37° C. for 72 hours before cell growth was measured by incorporation of ³H-thymidine as described for in vitro cellular proliferation assays. Cell lysates were prepared at 48 and 72 hr post transfection for Western blot analyses as described above. Membranes were probed with total PDGFR alpha antibody (Cell Signaling Technology) and with ACTINO (Chemicon International).

### In Vivo Xenografts

Rh41, Rh41-807R cells were injected s.c. into nude mice to establish xenograft models. The tumor bear mice were treated with or without drugs at doses and schedules indicated in Figure legend. Tumor volume was measured to assess tumor growth inhibition.

### Example 1

### Method of Characterizing Acquired Resistance Models RH41-807R and RH41-MAB391R in Response to IGF-1R Inhibitors

Human rhabdomyosarcoma cell line Rh41 was chosen in this study to develop acquired resistance because it expresses IGF-1R (Huang et al., Cancer Res., 69(1):161-170 (Jan. 1, 2009)), the target of BMS-754807 and MAB391; and is sensitive to both drugs (Carboni et al., Proceedings of the 100th Annual Meeting of the American Association for Cancer Research, 2009 Apr. 18-22; Denver, Colo., Abstract No. 1742). The acquired-resistant Rh41-807R and Rh41-MAB391R cell lines were developed using a stepwise exposure to increasing concentrations of either IGF-1R/IR inhibitor BMS-754807 or IGF-1R antibody MAB391 for extended periods of time until a resistance plateau was reach. The sensitivity of the parental and both acquired resistant cell lines to either drug was characterized in cell proliferation experiments by ³H-thymidine incorporation assay. As shown in Table 1, parental Rh41 was sensitive to both BMS-754807 (IC₅₀=5 nM) and MAB391 (IC₅₀=0.1 nM); comparing to the parental, Rh41-807R showed an almost 162-fold resistance to BMS-754807 while the Rh41-MAB391R cell line was extremely resistance to MAB391 with greater than 10.000-fold increase in the observed IC₅₀. Furthermore, when Rh41-807R was out of drug selection for a period of 3-months of passages (Rh41-807Rout), it still had a significant level of resistance to BMS-754807, suggesting the resistance of Rh41-807R to BMS-754807 was persistent.

Interestingly, when BMS-754807 was tested in the Rh41-MAB391R model, it was relatively active and inhibited growth of Rh41-MAB391R to a comparable level as the parental cells; whereas MAB391 was unable to inhibit the growth of both Rh41-MAB391R and Rh41-807R even at >2 μM. In addition, when tested against multiple other small molecular inhibitors of IGF-1R, Rh41-807R demonstrated cross-resistance to all the IGF-1R inhibitors tested whereas Rh41-MAB391R was still relatively sensitive to these drugs (Table 1). These results suggested there maybe different mechanisms of resistance to BMS-754807 and to MAB-391, and BMS-574807 could potentially overcome the resistance to IGF-1R antibody therapy.

TABLE 1
Resistance characterization of 807R and MAB391R
models to IGF-1R multiple inhibitors.

| | IC50 (nM) | | |
|---|---|---|---|
| IGF1R inhibitors | Rh41 | Rh41-807R | Rh41-MAB391R |
| BMS-754807 | 5 | 811 | 27 |
| MAB-391 | 0.1 | >2000 | >2000 |
| BMS-536924 | 31 | >1000 | 90.2 ± 2.2 |
| BMS-830913 | 0.02 | 9 | 0.02 ± 0.036 |
| BMS-809800 | 2 | 55 | 1.29 |
| BMS-808931 | <1 | 28 | <0.64 |
| OSI IGF-1R inhibitor | 81 | 3424 | 42.3 |

The sensitivity is defined as IC₅₀, which is the drug concentration that produced a 50% growth inhibition compared with untreated controls in ³H-thymidine incorporation assays.

Interestingly, when Rh41-807R was tested against multiple cytotoxic agents such as TAXOL® and Gemcitabine, it had a very similar level of sensitivity as the parental line to cytotoxic agents as well as to mTOR inhibitor rapamycin (see Table 2).

TABLE 2
Comparison of the sensitivity to cytotoxic agents
and mTOR inhibitor in Rh41 and Rh41-807R cells.

| | | |
|---|---|---|
| | Rh41 | Rh41-807R |
| Agent | IC₅₀ (nM) | IC₅₀ (nM) |

mTOR inhibitor

| | | |
|---|---|---|
| Rapamycin | <0.32 | <0.32 |

Cytotoxic agent

| | | |
|---|---|---|
| TAXOL ® | 2.1 ± 0.4 | 1.8 ± 0.3 |
| Taxotere | 0.5 | 0.5 |
| Ixabepilone | 4 ± 1.4 | 3.4 ± 0.4 |
| Vinflunine | 28 ± 2.8 | 21 ± 3.0 |
| Gemcitabine | 1.1 ± 0.4 | 3 ± 0.7 |

The sensitivity is defined as IC₅₀, which is the drug concentration that produced a 50% growth inhibition compared with untreated controls in ³H-thymidine incorporation assays.

### Example 2

### Methods of Assessing IGF-1R Expression and Activity in Relation to Acquired Resistance to IGF-1R Inhibition

Because both BMS-754807 and MAB391 target the IGF-1R function, the inventors investigated whether IGF-1R was involved in the mechanisms of acquired resistance. In comparison to the sensitive parental, Rh41-807R had significant down regulation of IGF-1R at both the RNA and protein levels (FIGS. 1A, 1B); whereas resistant Rh41-MAB391R maintained a similar expression level of IGF-1R RNA transcript (FIG. 1A), but had decreased level of IGF-1R protein (FIG. 1B). To further explore why MAB391R had inconsistent results in RNA and protein expression levels compared to the parental line, a wash out experiment was conducted. The result indicated that the IGF-1R protein level started to recover 2-hr after MAB391 was removed and reached the level of parent cells 24-hr post washout as measured by both western blot (FIG. 1C) and flow cytometry analyses (FIG. 1D). MAB391 can cause IGF-1R internalization and therefore decreases IGF-1R protein level on the cell surface in Rh41-MAB391R model. Because IGF-1R RNA level remained the same as the parental, this explained why after MAB391 removal, the IGF-1R protein expression on cell surface was restored fairly quickly in MAB391R cells due to RNA continuously the made available.

In addition, both IGF-2 and IGF2R were also down regulated in 807R cells but not in MAB391R cells. The decreased level for both genes was also observed in 807Rout cells (FIG. 6A).

Furthermore, the inventors also investigated whether there is any difference between sensitive Rh41S, resistant MAB391R and 807R in responsiveness to IGF-1 induced IGF-1R phosphorylation and activation of downstream pathway efforts such as pAKT and pERK. As shown in FIG. 1E, none of the cell lines showed any detectable IGF-1R phosphorylation in the absence of IGF-1, but they all responded to IGF-1 stimulation by increasing pIGF-1R, pAKT and pERK. Both Rh41-807R and Rh41-MAB391R cells seem to have higher basal level of pERK1/2 than the parental indicating higher basal activation of signaling in resistant models. In Rh41-807R, pAKT appeared to be less responsive to IGF-1 stimulation compared to both Rh41-MAB391R and the parental suggesting AKT was not activated through IGF signaling in Rh41-807R.

### Example 3

### Methods of Assessing the Shared and Unique Gene Expression Alterations Between RH41-807R and RH41-MAB391R Cell Lines

To explore the molecular differences between the sensitive and acquired resistant models, gene expression profiling was performed using Affymetrix GENECHIP®s. Statistical analyses of gene expression profiles identified two gene lists: one differentially expressed in Rh41-807R vs. parental, another differentially expressed in Rh41-MAB391R vs. parental, respectively. Overall, there were more genes with changed expression level in Rh41-807R than in Rh41-MAB391R cells when both were compared to the sensitive parental line. Cross comparison of the two analyses (FIG. 2A) identified genes that changed expression levels uniquely in either resistant model (genes in E, F, G and H sections of FIG. 2A), as well as four types of overlapped genes (A, B, C and D sections of FIG. 2A) which exhibited different expression patterns as illustrated in FIG. 3B. Clusters A and D were groups of genes with the same expression pattern change in both 807R and MAB391R, which may contribute to the common mechanisms of acquired resistance to agents targeting IGF-1R. These genes are listed in Table 3, of which, 25 probe sets (Cluster D) representing 21 unique genes were up-regulated in both resistant models; and 37 probe sets (Cluster A) representing 32 unique genes were down-regulated in both resistant models. Conversely, Cluster B and C were genes with opposite expression pattern changes in 807R and MAB391R, and listed in Table 4.

Furthermore, the gene expression analysis was performed on the 807R cells that were out of BMS-754807 selection for at least 3-months (807Rout) but still remained the resistance to the drug. Overall, the expression pattern was very similar in both 807R and 807Rout; and there were very limited number of genes restored their expression levels to that of the sensitive parental cells after the drug was removed (FIG. 2B).

Since Rh41-807R and Rh41-MAB391R both are resistant to multiple IGF-1R inhibitors, genes with the same expression pattern change in both 807R and MAB391R, which may contribute to the common mechanisms of acquired resistance to agents targeting IGF-1R. Those genes were involved in cell signaling pathways (e.g., FGF9, PDGFR-α and DUSP13), cell matrix interactions (e.g., MMP2, MMP3 and TIMP3), cell cycle regulation (e.g., DCX, PLAU, AMACR and MXI1), and apoptosis (e.g., PAWR, PAX3 and TMSB4X). Compared to sensitive parental cells, both 807R and MAB391R cells had MMP2, TIMP3 and FGF9 up regulated, whereas SNRPN, TFAP2B, MMP3 and PLAU were down regulated (Table 3).

Interestingly, there were genes with opposite expression pattern (Cluster B and C in FIG. 2B) between 807R and MAB391R cells, those are exampled by AXL, FADS3, MME, NNMT, PLXNC1, MYOZ2, EPHA3 and CDH2 (Table 4). There were also genes that are uniquely changed expression only in MAB391R (DLK1, TUSC3 and VCAN) but not in 807R (FIG. 6B); and only upregulated in 807R (FHL1, EEF1A2, PRRX1, GHR; FIG. 6C) or down-regulated in 807R (INSIG1, CCND1, CCND2, ERBB3; FIG. 6D) but no change in MAB391R.

The second group of genes had opposite expression changes between Rh41-807R and Rh41-MAB391R cells. Genes such as AXL, FADS3, MME, NNMT, and PLXNC1 were up-regulated in Rh41-MAB391R but down-regulated in Rh41-807R, whereas MYOZ2, EPHA3 and CDH2genes had converse patterns.

The third group of genes showed expression changes only in one resistant model. For example, DLK1 was up-regulated; TUSC3 and VCAN were down regulated only in MAB391R not in Rh41-807R. FHL1, EEF1A2, PRR^{x} and GHR were up-regulated; INSIG1, CCND1, CCND2, ERBB3 were down regulated uniquely in Rh41-807R.

These genes may contribute to differential resistance mechanisms either to BMS-754807 or to MAB391.

Pathway analyses were done for the genes that are uniquely differential expressed in 807R and MAB391R cells. The selected top biological functions and canonical pathways are compared and shown in FIG. 2C. Clearly, the genes uniquely up-regulated in either 807R or MAB391R cells contribute to the common molecular and cellular functions; however they have significant difference in the involvement of different canonical pathways such as IGF-1, PTEN, PDGF or EGF signaling.

Overall, expression patterns were similar in both Rh41-807R and Rh41-807Rout. Only a limited number of genes in Rh41-807Rout showed restored expression levels similar to parental Rh41 cells, suggesting gene expression patterns in Rh41-807R persisted even after the drug were removed.

TABLE 3
Genes with the same expression pattern change in both 807R
and MAB391R, which may contribute to the common mechanisms
of acquired resistance to agents targeting IGF-1R.

| | | p-value | Fold-Change | p-value | Fold-Change |
|---|---|---|---|---|---|
| | | (807R vs. | (807R vs. | (MAB391R vs. | (MAB391R vs. |
| Probe | Gene Symbol | Rh41S) | Rh41S) | Rh41S) | Rh41S) |
| 209539_at | ARHGEF6 | 2.55E−05 | 8.2 | 1.25E−03 | 2.2 |
| 203296_s_at | ATP1A2 | 2.65E−03 | 3.0 | 2.28E−03 | 3.2 |
| 218309_at | CAMK2N1 | 1.07E−03 | 2.6 | 2.41E−03 | 2.1 |
| 221773_at | ELK3 | 2.39E−04 | 3.3 | 1.70E−03 | 2.0 |
| 206404_at | FGF9 | 5.96E−04 | 5.2 | 3.94E−03 | 2.7 |
| 203066_at | GALNAC4S-6ST | 4.91E−04 | 2.5 | 3.56E−04 | 2.7 |
| 212510_at | GPD1L | 1.27E−04 | 2.5 | 7.17E−05 | 2.9 |
| 206132_at | MCC | 2.07E−02 | 2.0 | 1.99E−02 | 2.0 |
| 210794_s_at | MEG3 | 1.71E−04 | 6.7 | 1.86E−03 | 2.8 |
| 201069_at | MMP2 | 9.55E−04 | 3.3 | 1.31E−03 | 3.0 |
| 212096_s_at | MTUS1 | 1.04E−03 | 2.3 | 4.07E−04 | 2.9 |
| 202364_at | MXI1 | 2.57E−05 | 2.7 | 6.17E−05 | 2.2 |
| 204004_at | PAWR | 3.49E−04 | 2.6 | 4.48E−04 | 2.4 |
| 207680_x_at | PAX3 | 2.15E−02 | 2.8 | 2.63E−02 | 2.6 |
| 203131_at | PDGFRA | 2.62E−09 | 89.0 | 1.46E−06 | 2.5 |
| 203355_s_at | PSD3 | 1.75E−03 | 2.2 | 1.25E−03 | 2.4 |
| 208131_s_at | PTGIS | 9.77E−03 | 2.5 | 3.54E−03 | 3.4 |
| 210702_s_at | PTGIS | 7.58E−03 | 2.0 | 1.19E−03 | 3.2 |
| 211892_s_at | PTGIS | 2.70E−03 | 2.4 | 5.99E−03 | 2.0 |
| 206805_at | SEMA3A | 1.64E−03 | 2.9 | 3.49E−03 | 2.4 |
| 212956_at | TBC1D9 | 8.28E−07 | 3.3 | 1.90E−06 | 2.6 |
| 201147_s_at | TIMP3 | 2.81E−03 | 2.2 | 1.14E−04 | 6.1 |
| 201149_s_at | TIMP3 | 4.05E−03 | 2.1 | 2.14E−04 | 5.0 |
| 201150_s_at | TIMP3 | 3.31E−04 | 3.2 | 6.36E−05 | 5.8 |
| 216438_s_at | TMSB4X /// | 1.82E−03 | 2.6 | 9.49E−04 | 3.1 |
| | TMSL3 | | | | |
| 220988_s_at | AMACR /// | 3.93E−04 | −3.9 | 2.76E−04 | −4.5 |
| | C1QTNF3 | | | | |
| 214357_at | C1orf105 | 6.82E−07 | −57.2 | 5.28E−04 | −2.1 |
| 207317_s_at | CASQ2 | 9.00E−03 | −2.3 | 5.35E−04 | −5.8 |
| 213492_at | COL2A1 | 2.73E−04 | −4.6 | 2.13E−04 | −5.1 |
| 209687_at | CXCL12 | 6.43E−04 | −2.3 | 4.79E−04 | −2.4 |
| 204850_s_at | DCX | 7.04E−04 | −6.2 | 1.73E−02 | −2.1 |
| 206061_s_at | DICER 1 | 5.90E−05 | −4.3 | 5.68E−04 | −2.3 |
| 212888_at | DICER 1 | 1.02E−04 | −3.9 | 5.22E−04 | −2.4 |
| 213229_at | DICER 1 | 1.11E−03 | −5.0 | 1.81E−02 | −2.1 |
| 216260_at | DICER 1 | 2.71E−04 | −4.3 | 4.10E−04 | −3.7 |
| 219963_at | DUSP13 | 1.56E−03 | −3.9 | 1.31E−02 | −2.1 |
| 208770_s_at | EIF4EBP2 | 1.92E−04 | −2.3 | 3.78E−04 | −2.0 |
| 220615_s_at | FAR2 | 1.04E−03 | −2.3 | 3.76E−04 | −3.0 |
| 205201_at | GLI3 | 1.11E−02 | −2.3 | 6.55E−03 | −2.6 |
| 205184_at | GNG4 | 3.44E−03 | −2.1 | 1.65E−03 | −2.5 |
| 215121_x_at | IGL@ /// | 5.60E−03 | −2.8 | 4.53E−04 | −7.3 |
| | IGLC2 /// | | | | |
| | IGLV2-14 | | | | |
| 213447_at | IPW | 2.83E−06 | −4.5 | 3.06E−06 | −4.4 |
| 206012_at | LEFTY2 | 1.28E−04 | −4.6 | 8.47E−05 | −5.4 |
| 207079_s_at | MED6 | 9.23E−05 | −2.4 | 1.61E−04 | −2.1 |
| 212713_at | MFAP4 | 1.32E−05 | −4.4 | 5.91E−05 | −2.7 |
| 205828_at | MMP3 | 1.94E−05 | −13.7 | 1.50E−05 | −16.3 |
| 209888_s_at | MYL1 | 1.08E−03 | −6.1 | 3.01E−02 | −2.0 |
| 209550_at | NDN | 1.79E−03 | −5.5 | 4.26E−03 | −3.9 |
| 205872_x_at | PDE4DIP | 2.31E−03 | −2.8 | 4.35E−03 | −2.4 |
| 219304_s_at | PDGFD | 1.60E−02 | −2.0 | 1.17E−02 | −2.2 |
| 209621_s_at | PDLIM3 | 1.12E−03 | −3.8 | 4.88E−03 | −2.5 |
| 205479_s_at | PLAU | 3.98E−03 | −3.6 | 1.86E−02 | −2.3 |
| 209598_at | PNMA2 | 9.19E−08 | −6.1 | 2.18E−06 | −2.3 |
| 220454_s_at | SEMA6A | 9.88E−03 | −3.1 | 3.53E−02 | −2.2 |
| 211115_x_at | SIP1 | 4.91E−04 | −2.3 | 9.73E−04 | −2.0 |
| 219090_at | SLC24A3 | 3.37E−03 | −3.5 | 9.88E−03 | −2.6 |
| 57588_at | SLC24A3 | 8.63E−04 | −2.1 | 6.86E−04 | −2.2 |
| 201522_x_at | SNRPN /// | 1.02E−08 | −112.6 | 2.24E−08 | −48.2 |
| | SNURF | | | | |
| 206042_x_at | SNRPN /// | 1.47E−06 | −34.5 | 2.42E−06 | −22.8 |
| | SNURF | | | | |
| 204743_at | TAGLN3 | 1.71E−04 | −2.7 | 1.22E−04 | −2.9 |
| 214451_at | TFAP2B | 9.80E−05 | −6.1 | 1.46E−03 | −2.5 |
| 208195_at | TTN | 1.34E−02 | −3.7 | 5.18E−03 | −5.5 |

The genes were identified by t-test to compare the gene profile of either 807R or MAB391R to that of the sensitive parental to select the ones with p < 0.05 and fold change > 2 in both comparisons.

TABLE 4
Genes with the opposite expression pattern change in both 807R and MAB391R,
which may contribute to the differential mechanisms of acquired resistance
to small molecular inhibitor BMS-754807 or to anti-IGF-1R antibody MAB391.

| | | p-value | Fold-Change | p-value | Fold-Change |
|---|---|---|---|---|---|
| | | (807R vs. | (807R vs. | (MAB391R vs. | (MAB391R vs. |
| Probe | Gene Symbol | Rh41S) | Rh41S) | Rh41S) | Rh41S) |
| 204174_at | ALOX5AP | 7.79E−03 | 2.6 | 2.50E−03 | −3.7 |
| 218870_at | ARHGAP15 | 5.72E−03 | 2.1 | 1.56E−03 | −3.0 |
| 220359_s_at | ARPP-21 | 2.16E−03 | 2.5 | 5.65E−03 | −2.0 |
| 218332_at | BEX1 | 2.61E−03 | 2.1 | 3.44E−05 | −9.9 |
| 211518_s_at | BMP4 | 4.26E−04 | 3.8 | 2.64E−03 | −2.3 |
| 203440_at | CDH2 | 1.42E−03 | 2.3 | 1.48E−04 | −4.6 |
| 218094_s_at | DBNDD2 /// | 6.22E−03 | 2.6 | 1.50E−02 | −2.1 |
| | SYS1-DBNDD2 | | | | |
| 208399_s_at | EDN3 | 1.90E−03 | 2.6 | 4.22E−04 | −4.0 |
| 206070_s_at | EPHA3 | 1.90E−03 | 6.5 | 3.30E−02 | −2.3 |
| 203274_at | F8A1 /// F8A2 /// | 2.37E−03 | 2.3 | 6.24E−04 | −3.2 |
| | F8A3 | | | | |
| 212737_at | GM2A | 5.51E−03 | 2.1 | 5.36E−03 | −2.1 |
| 204298_s_at | LOX | 2.70E−02 | 2.6 | 4.59E−02 | −2.2 |
| 202729_s_at | LTBP1 | 1.16E−03 | 2.0 | 3.26E−04 | −2.6 |
| 207148_x_at | MYOZ2 | 2.85E−04 | 4.9 | 2.49E−04 | −5.2 |
| 213782_s_at | MYOZ2 | 3.43E−04 | 4.7 | 1.44E−04 | −7.0 |
| 206089_at | NELL1 | 1.27E−03 | 3.3 | 1.91E−03 | −3.0 |
| 201037_at | PFKP | 8.52E−04 | 3.3 | 2.79E−03 | −2.4 |
| 211380_s_at | PRKG1 | 2.36E−04 | 4.1 | 1.05E−03 | −2.6 |
| 214433_s_at | SELENBP1 | 1.15E−02 | 2.1 | 9.49E−03 | −2.2 |
| 205352_at | SERPINI1 | 3.79E−03 | 2.3 | 1.93E−03 | −2.7 |
| 204042_at | WASF3 | 2.42E−05 | 4.0 | 1.10E−04 | −2.6 |
| 200965_s_at | ABLIM1 | 2.58E−03 | −2.1 | 4.83E−04 | 3.2 |
| 208353_x_at | ANK1 | 1.28E−03 | −3.5 | 1.08E−02 | 2.0 |
| 202686_s_at | AXL | 3.42E−05 | −4.9 | 1.23E−05 | 7.8 |
| 221211_s_at | C21orf7 | 7.00E−03 | −2.4 | 1.41E−03 | 3.9 |
| 202965_s_at | CAPN6 | 5.15E−05 | −8.7 | 9.43E−04 | 2.8 |
| 210026_s_at | CARD10 | 4.43E−04 | −5.5 | 1.21E−02 | 2.0 |
| 203324_s_at | CAV2 | 1.40E−02 | −2.8 | 1.20E−02 | 2.9 |
| 201289_at | CYR61 | 8.35E−04 | −2.2 | 7.11E−05 | 4.5 |
| 210764_s_at | CYR61 | 2.78E−04 | −3.0 | 1.03E−04 | 4.1 |
| 218660_at | DYSF | 7.48E−04 | −3.0 | 3.10E−04 | 3.9 |
| 204257_at | FADS3 | 1.49E−03 | −2.9 | 5.10E−04 | 4.0 |
| 216080_s_at | FADS3 | 1.22E−04 | −2.5 | 2.19E−05 | 4.2 |
| 203434_s_at | MME | 1.20E−03 | −3.9 | 1.00E−03 | 4.2 |
| 203435_s_at | MME | 3.88E−05 | −2.3 | 3.84E−06 | 4.4 |
| 202237_at | NNMT | 2.47E−05 | −14.2 | 1.35E−04 | 5.6 |
| 202238_s_at | NNMT | 4.53E−04 | −5.9 | 2.86E−04 | 7.4 |
| 218162_at | OLFML3 | 1.17E−04 | −2.4 | 1.63E−04 | 2.3 |
| 206470_at | PLXNC1 | 3.00E−04 | −2.2 | 2.38E−04 | 2.4 |
| 213241_at | PLXNC1 | 3.78E−03 | −4.3 | 2.60E−02 | 2.3 |
| 215389_s_at | TNNT2 | 5.34E−06 | −12.6 | 4.28E−04 | 2.3 |
| 211177_s_at | TXNRD2 | 4.45E−04 | −3.2 | 7.91E−04 | 2.7 |

The genes were identified by t-test to compare the gene profile of either 807R or MAB391R to that of the sensitive parental to select the ones with p < 0.05 and fold change > 2 in both comparisons.

### Example 4

### Methods of Assessing DNA Copy Number Changes Associated with Acquired Resistance to BMS-754807 and/or MAB391

DNA copy number analysis was performed on Rh41-807R, Rh41-MAB391R and the parental Rh41 cell lines using SNP-chip for genomic abnormalities. Compared to sensitive Rh41 cell, a number of low-level copy number differences (gain or loss) were observed in resistant Rh41-807R cells (Table 5), whereas only a few copy number gains were observed in Rh41-MAB391R cells. For example, as illustrated in FIG. 9, both Rh41 and Rh41-MAB391R had mostly normal copy number on chromosome 4 in q11-q21 region; however, Rh41-807R had genomic abnormalities in this region. Compared to Rh41 cells, Rh41-807R harbored amplification of chr4q11-q12 containing c-KIT and PDGFR-α genes with 1.9- and 3.3-fold gain over Rh41 copy number, respectively (FIG. 2D). Gene amplification is consistent with overexpression of these two genes in Rh41-807R cells (FIGS. 3A, 3C). Another interesting observation is that PTEN loss was found in Rh41-807R compared to Rh41 cells (1.5×lower) (Table 5), which is consistence with decreased gene expression level in Rh41-807R cells (1.8-fold reduction, p=0.018).

The inventors then performed a gene-chromosomal enrichment analysis by looking at the number of genes on each chromosome that had significant changes in expression levels (p<0.05 and fold change>2 in t-test) either in Rh41-807 or in Rh41-MAB391R vs. the sensitive parental and then compared the latter to the number of genes located on each chromosome that are presented on the gene chip to see if a particular chromosome or any particular regions on a chromosome had a higher percentage of genes expression level changes due to resistance to IGF-1R inhibitors. As shown in (Table 6), the gene enrichment was seen on several chromosomes with significance of p<0.01 in Fisher-exact test. For example, in 807R cells, genes with upregulated expression are enriched on chromosome 4, which is in consistence with DNA amplifications seen on chromosome 4 (FIG. 2D and FIG. 7).

TABLE 5
Summary of Genomic Abnormality in
Cell Line Resistance to BMS-754807

| | | | |
|---|---|---|---|
| | | | Ratio |
| | | | (Rh41- |
| Altered | Positions in | Selected genes in altered | 807R vs. |
| regions | chromosome | genomic regions | Rh41S) |

Amplified in Rh41-807R

| | | | |
|---|---|---|---|
| 1q21 | 150.1 Mb-150.3 Mb | S100A10, S100A11 | 1.52 |
| 1q22-1q23 | 158 Mb-160 Mb | DUSP23, ATF6, | 1.54 |
| | | DUSP12, FCGR2A, | |
| | | FCGR2B, FCGR2C; | |
| | | FCRLA, FCRLB | |
| 1q24 | 163.6 Mb-163.9 Mb | ATF6, RXRG | |
| 2p22 | 39.7 Mb-41.2 Mb | TMEM178, SLC8A1 | |
| 4q12-4q21 | 53 Mb-82 Mb | PDGFRA, RASL11B, | 3.31 |
| | | LNX1 | |
| | | KIT, KDR, IGFBP7 | 1.91 |
| | | EPHA5 | 1.55 |
| | | TMPRSS11A, | 1.69 |
| | | TMPRSS11B, | |
| | | TMPRSS11D | |
| | | TMPRSS11E, | 1.54 |
| | | TMPRSS11E2, | |
| | | TMPRSS11F | |
| | | EREG, AREG, AREGB | 1.54 |
| | | RASSF6, G3BP2 | 1.54 |
| | | FGF5 | 1.59 |
| 4q28 | 128 Mb-129.3 Mb | PLK4 | 1.66 |
| 4q31.2-4q32 | 154.7 Mb-161 Mb | PDGFC, GUCY1A3, | 1.73 |
| | | GUCY1B3, RAPGEF2 | |
| 4q33 | 171.2 Mb-171.4 Mb | AADAT | 1.69 |
| 4q34 | 172.6 Mb-173.5 Mb | GALNTL6 | 1.68 |
| 4q34 | 174.5 Mb-176 Mb | HPGD | 2.10 |
| 12q13 | 48.5 Mb-49 Mb | FAIM2, TMBIM6 | 2.00 |
| 17p13 | 11.3 Mb-11.5 Mb | MYH4, MYH8 | 1.52 |

Deleted in Rh41-807R

| | | | |
|---|---|---|---|
| 3p26 | 3.5 Kb-349 Kb | LOC642891 | 0.66 |
| 3p25-3p24 | 13.6 Mb-32 Mb | NR2C2, RAB5A, | 0.65 |
| | | SH3BP5, NKIRAS1, | |
| | | RAB5A, RARB, | |
| | | TOP2B, TGFBR2 | |
| 3p14-3p12 | 59.7 Mb-81.5 Mb | PTPRG, PSMD6, | 0.65 |
| | | FOXP1 | |
| 10q22-10q24 | 73.8 Mb-102 Mb | PLAU | 0.33 |
| | | SPOCK2, PLA2G12B, | 0.55 |
| | | PPP3CB | |
| | | DUSP13, DUPD1 | 0.64 |
| | | PTEN, PAPSS2, | 0.66 |
| | | PPP1R3C | |
| | | FGFBP3, IDE | 0.56 |
| | | PIK3AP1, PI4K2A | 0.66 |

TABLE 6
Gene-chromosomal enrichment analysis.

| Description | p value in Fisher-test |
|---|---|
| 807R_up_chr4 | 2.34E−09 |
| 807R_up_chr6 | 2.26E−04 |
| 807R_up_chr8 | 6.95E−05 |
| 807R_up_chr11 | 2.26E−04 |
| 807R_up_chr14 | 1.51E−03 |
| 807R_up_chr15 | 8.06E−04 |
| 807R_up_chr23 | 2.00E−42 |
| 807R_down_chr5 | 9.48E−03 |
| 807R_down_chr10 | 8.85E−16 |
| 807R_down_chr14 | 4.60E−06 |
| 807R_down_chr19 | 1.38E−05 |
| 807R_down_chr23 | 4.61E−05 |
| Mab391R_up_chr17 | 1.64E−03 |
| Mab391R_down_chr5 | 9.20E−03 |
| Mab391R_down_chr14 | 5.45E−03 |

The number of genes that are significantly changed expression level (p < 0.05 and fold change > 2 in t-test) either in Rh41-807 or in Rh41-MAB391R on each chromosome were comparing to the total number of genes located on each chromosome that are presented on the gene chip. The significance was derived from Fisher-exact test.

### Example 5

### Methods of Further Assessing RH41-807R and RH41-MAB391R Expression of Unique Alternative Pathway Genes and Determining Whether Such Expression Provides the Cell Lines with Different Resistance Mechanisms

Comparing genes expression profiles, the inventors found there were some key signaling pathways that were differentially expressed in Rh41-807R and Rh41-MAB391R cells. An interesting example was PDGFR-α, which had >89 fold increase in RNA expression in Rh41-807R cells but not in Rh41-MAB391R compared to the parental; and the induction of PDGFR-α was persistent even in cells out of BMS-754807 selection for 3 months (FIG. 3A). The expression pattern was also confirmed at protein levels (FIG. 3A); PDGFR-α was also constitutively activated in Rh41-807R cells (FIG. 3B). In addition, the inventors found that when Rh41 sensitive parental cell treated with BMS-754807, the induction of PDGFR-α was dose-dependent and common for several IGF-1R inhibitors tested; and the increased expression was an early event and was seen as early as 4 hrs after treatment with IGF-1R inhibitors (FIG. 3F and FIG. 3G). Other genes such as c-KIT, FGFR2 and EPHA3 had similar expression pattern as PDGFR-α (FIG. 3C). Several Eph receptor family members such as EPHA3, EPHA4 and EPHB2 as well as ligand EFNA3 were overexpressed in 807R. A large member of RAS/RAF related genes (e.g., ARAF, ARHGAP8, RAB40B, RAB6B, RAB7L1, RASL11B, RASSF2, RHOC and RHOQ) were also upregulated only in 807R cells (data not shown).

Another interesting example was AXL which exhibited an expression pattern that was opposite in each cell line. Specifically, AXL was expressed 7.8 fold higher in MAB391R cells, but was 4.9 fold lower in 807R cells compared to the sensitive Rh41S (FIG. 3D). Western Blot analysis further confirmed that AXL protein expression also has higher level in MAB391R compared to the sensitive parental cells (FIG. 3D). DLK1 is another gene uniquely increased expression only in MAB391R not in 807R cells (FIG. 6B).

### Example 6

### Methods of Assessing PDGFR-α Activity or Expression Modulation Changes In Vitro Response of RH41-807R to BMS-754807

Because Rh41-807R cells had increased PDGFR-α expression and were constitutively activated (FIGS. 3A and 3B), the inventors sought to test whether dovitinib, a drug that has inhibitory activity against PDGFR, was active in the resistant cells. When both Rh41-807R and parental Rh41 cells were treated with dovitinib, Rh41-807R was more sensitive to dovitinib than Rh41 (FIG. 4A), suggesting PDGFR may play a role in the resistance to BMS-754807. However, dovitinib has selectivity against other kinases such as VEGFR, c-KIT, FGFR and FLT beside PDGFR (Homsi, J. et al., “Spectrum of activity and mechanism of action of VEGF/PDGF inhibitors”, Cancer Control, 14:285-294 (2007)); and Rh41-807R cells also had increased expression of c-KIT and FGFR2 in addition to PDGFR-α (FIG. 3C), which could also contribute to increased sensitivity of dovitinib in Rh41-807R by targeting multiple kinases. The inventors then further tested several compounds (axitinib, sorafenib and sunitinib) that have activity against PDGFR. Various degrees of growth inhibitory activity was observed in Rh41-807R cells (Table 7) suggesting PDGFR could be one of factors that were driving resistance to BMS-754807.

To dissect out and gain a better understanding whether there was a direct correlation between the overexpression of PDGFR-α and the acquisition of BMS-754807 resistance, the inventors sought to suppress the expression of PDGFR-α using small interfering RNA (siRNA)-mediated knockdown of PDGFR-α in Rh41-807R cells. The results showed expression of PDGFR-α was efficiently suppressed at 48 hours after transfection with siRNA to PDGFR-α, but no effect was observed with control siRNA (FIG. 4B). The inventors next looked at the effect of PDGFR-a suppression on response to BMS-754807 by in vitro cell proliferation assay. Both Rh41S and Rh41-807R cells were transfected with either control siRNA or PDGFR-a-specific siRNA, then were treated with different concentrations of BMS-754807 for 48 hrs. In this experiment, the inventors found that Rh41 sensitive parental cells were still sensitive with no significant differences in the sensitivity to BMS-754807 between untransfected, control siRNA and PDGFR-α-specific siRNA transfected cells (FIG. 4B and FIG. 8A) probably due to very low level of PDGFR-α expression (FIG. 3A). However, Rh41-807R cells treated with PDGFR-α siRNA were sensitive to growth inhibition by BMS-754807 and Rh41-807R cells responded essentially the same as the sensitive parental cells, whereas cells treated with control siRNA were not (FIG. 4B and FIG. 8B). These results provided evidence that overexpression of PDGFR-α confers BMS-754807 resistance, and down-regulation of PDGFR-α in Rh41-807R cells restores the sensitivity to BMS-754807.

TABLE 7
Comparison of the Sensitivity to PDGFR
Inhibitors in Rh41S and Rh41-807R Cells

| | | Rh41 | Rh41-807R |
|---|---|---|---|
| TKI | Selectivity | IC₅₀ (nM) | IC₅₀ (nM) |
| Dovitinib | VEGFR/PDGFR/c-KIT/FGFR/ | 6456 ± 60 | 722 ± 264 |
| | FLT | | |
| Axitinib | VEGFR/PDGFR/c-KIT | 744 ± 178 | 218 ± 88 |
| Sorafenib | VEGFR/PDGFR/RAF | >2000 | 804 ± 210 |
| Sunitinib | VEGFR/PDGFR/c-KIT/FLT | 1403 ± 634 | 1022 ± 321 |

### Example 7

### Methods of Assessing Whether RH41-807R and RH41-MAB391R Xenograft Tumor In Vivo Retain the Expression Pattern of IGF-1R and PDGFRA AS was Observed In Vitro

To test whether Rh41-807R is also resistant to BMS-754807 in vivo, both Rh41 and Rh41-807R xenograft tumors were established. Rh41 achieved 73% tumor growth inhibition (TGI) in response to BMS-754807, while Rh41-807R had only 35% TGI, indicating the resistance was consistent with the in vitro observations (FIG. 10). When examining the expression of IGF-1R and PDGFR-α in the xenograft tumors, the inventors found the reduced IGF-1R and increased PDGFR-α protein expression in Rh41-807R than in the parental were still retained (FIG. 3E). The c-KIT, FGFR2 and PDGFR-α also had higher RNA expression levels in Rh41-807R tumor compared to that in Rh41-S tumors, this is in consistence with the pattern observed in cell lines (FIG. 9), suggesting the mechanisms of resistance to BMS-754807 were kept the same both in vitro and in vivo.

### Example 8

### Methods of Assessing Whether Synergistic Activity Exists for the Combination of an PDGFR Antagonist with an IGF-1R Antagonist

The inventors then investigated the effect of combining BMS-754807 with PDGFR antagonists in Rh41-807R cells and found that BMS-754807 plus dovitinib, sunitinib, imatinib or axitinib (FIG. 5) all had synergistic activity in growth inhibition. This indicated inhibition of both IGF-1R and PDGFR exhibited synergistic activity, and were both necessary to achieve improved response.

### Example 9

### Methods of Further Characterizing the Resistance of Multiple Models to the IGF-1R Inhibitor BMS-754807

### Materials and Methods

Four cell lines of different tumor types, MCF7, Rh41, Rh1, Geo and SW480 (breast, sarcomas and colon), were induced to develop acquired resistance to BMS-754807 by stepwise exposure to increasing concentrations of the drug for extended periods. Analyses of in vitro and in vivo drug response, gene expression profiles, signaling pathways and gene copy numbers were performed to characterize the resistant models and the corresponding sensitive parental cells.

The sensitivity of multiple acquired resistant cell lines and the corresponding parental lines to GF-1R inhibitor BMS-754807 were characterized in cell proliferation experiments by ³H-thymidine incorporation assay and defined as IC₅₀ value which is the drug concentration that produced a 50% growth inhibition compared with untreated controls. The relative resistance was defined as fold changes in IC₅₀ in resistant cells compared with the parent cells and varied depending upon the cell model, with Rh41-807 demonstrating the most resistance compared to the sensitive parental line (Table 8). The resistant models also showed cross-resistance to other IGF 1R inhibitors (Table 9).

TABLE 8

| | | BMS-754807 | | |
|---|---|---|---|---|
| | Parental | Maintenance | IC50 | Relative |
| Cell Line | IC50 (nM)¹ | Conc. | (nM)¹ | resistance² |
| MCF-7 | 16 | 100 | 735 | 46X |
| Rh1 | 27 | 120 | 1795 | 66.5X |
| Rh41 | 5 | 600 | 811 | 162X |
| SW403 | 220 | 500 | 979 | 4.5X |
| Geo | 350 | 2000 | 1231 | 3.5X |

TABLE 9

| | IGF-1R inhibitor Compound (IC₅₀) | | | |
|---|---|---|---|---|
| | BMS- | BMS- | BMS- | BMS- |
| Cell line | 754807 | 830913 | 809800 | 874248 |
| MCF-7 | 16 | 2 ± 1 | 14 ± 2 | 543 |
| MCF-7-807R | 972 ± 400 | ND | ND | ND |
| Rh1 | 27 | 9 ± 4 | 71 ± 27 | >1000 |
| Rh1-807R | 1795 ± 1387 | 20 ± 14 | 596 | >5000 |
| Rh41 | 5 | <0.32 | 2 ± 1 | 81 |
| Rh41-807R | 811 | 9 ± 6 | 55 ± 33 | 3424 |
| SW403 | 220 | 1.2 ± 0.6 | 5 ± 2 | 79 |
| SW403-807R | 979 ± 528 | 7 ± 3 | 66 ± 41 | NT |
| Geo | 350 | 4 ± 3 | 16 ± 7 | 963 |
| GEO-807R | 1231 ± 251 | 29 ± 16 | 214 ± 74 | >1000 |

### Gene Expression Alterations in BMS-754807 IGF-1R Inhibitor Resistant Models

To explore the molecular differences between the sensitive and acquired resistant models, gene expression profiling was performed using Affymetrix GENECHIP®s. Statistical analyses of gene expression profiles for each cell line pair identified genes either significantly up-regulated or down-regulated in acquired resistant cells compared to the corresponding sensitive parental (p<0.05 and 2-fold in t-test). When cross-compared five cell line pairs, the inventors identified two gene lists: one is up-regulated in at least 3 out 5 resistant models (Table 10); another is down-regulated in at least 3 out 5 resistant models (Table 11), respectively.

TABLE 10
Genes Upregulated in at least 3 out of
5 IGF-1R Inhibitor Resistant Models

| Gene Description | Gene Symbol |
|---|---|
| cyclin G2 | CCNG2 |
| CD55 molecule, decay accelerating factor for | CD55 |
| complement | |
| D site of albumin promoter (albumin D-box) | DBP |
| binding protein | |
| GABA(A) receptor-associated protein | GABARAPL1 /// |
| like 1 /// 3 | GABARAPL3 |
| GM2 ganglioside activator | GM2A |
| GULP, engulfment adaptor PTB domain | GULP1 |
| containing 1 | |
| Kruppel-like factor 4 (gut) | KLF4 |
| MAX interactor 1 | MXI1 |
| plasminogen activator, urokinase receptor | PLAUR |
| proline rich Gla (G-carboxyglutamic acid) 1 | PRRG1 |
| protein tyrosine phosphatase, receptor type, R | PTPRR |
| spermidine/spermine N1-acetyltransferase 1 | SAT1 |
| TIMP metallopeptidase inhibitor 3 | TIMP3 |
| TCDD-inducible poly(ADP-ribose) polymerase | TIPARP |
| thioredoxin interacting protein | TXNIP |

TABLE 11
Genes Downregulated in at least 3 out
of 5 IGF-1R Inhibitor Resistant Models

| Gene Description | Gene Symbol |
|---|---|
| cyclin D2 | CCND2 |
| 3′-phosphoadenosine 5′-phosphosulfate synthase 2 | PAPSS2 |
| protein tyrosine phosphatase, receptor type, D | PTPRD |
| polymerase (DNA directed), epsilon 2 (p59 subunit) | POLE2 |
| fatty acid desaturase 1 | FADS1 |
| protein tyrosine phosphatase, receptor type, D | PTPRD |
| asparaginase like 1 | ASRGL1 |
| helicase, lymphoid-specific | HELLS |

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

The entire disclosure of each document cited (including patents, patent applications, journal articles, abstracts, laboratory manuals, books, GENBANK® Accession numbers, SWISS-PROT® Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

What is claimed is:

1. A method for predicting the likelihood a patient will respond therapeutically to a cancer treatment comprising the administration of an IGF-1R inhibitor, wherein said prediction method comprises the steps of:
(a) measuring the level of IGF-1R in a sample from said patient; and
(b) comparing the level of IGF-1R in said sample relative to a standard, wherein a increased expression level indicates an increased likelihood said patient will respond therapeutically to said cancer treatment.

2. A method for predicting the likelihood a patient will respond therapeutically to a cancer treatment comprising the administration of an IGF-1R inhibitor, wherein said prediction method comprises the steps of:
(a) measuring the level of a biomarker in a sample from said patient, wherein said biomarker is selected from the group consisting of: IGFBP3; IGFBP5, IGFBP6; AXL; and PDGFR-α; and
(b) comparing the level of said biomarker in said sample relative to a standard, wherein an increased expression level indicates a decreased likelihood said patient will respond therapeutically to said cancer treatment.

3. A method for treating a patient with cancer comprising the steps of:
(a) measuring the level of a IGF-1R in a sample from said patient; and
(b) comparing the level of IGF-1R in said sample relative to a standard, wherein an increased expression level indicates an increased likelihood said patient will respond therapeutically to a cancer treatment comprising the administration of an IGF-1R inhibitor.

4. A method for treating a patient with cancer comprising the steps of:
(a) measuring the level of a biomarker in a sample from said patient, wherein said biomarker is selected from the group consisting of: IGFBP3; IGFBP5, IGFBP6; AXL; and PDGFR-α; and
(b) comparing the level of said biomarker in said sample relative to a standard, wherein an increased expression level indicates a decreased likelihood said patient will respond therapeutically to a cancer treatment comprising the administration of an IGF-1R inhibitor.

5. A method of identifying a treatment regimen for a patient, comprising the steps of:
(a) measuring the level of IGF-1R in a sample from said patient; and
(b) comparing the level of IGF-1R in said sample relative to a standard, wherein a decreased expression level indicates a decreased likelihood said patient will respond therapeutically to a cancer treatment comprising the administration of an IGF-1R inhibitor, and recommending a more aggressive therapy.

6. The method according to claim 5, wherein said more aggressive therapy comprises a member of the group consisting of: (a) administering a higher dose of said cancer treatment; (b) administering said cancer treatment at an increased frequency; (c) administering said cancer treatment in combination with another therapy; and (d) administering said cancer treatment in combination with a PDGFR-α inhibitor.

7. A method of identifying a treatment regimen for a patient, comprising the steps of:
(a) measuring the level of a biomarker in a sample from said patient, wherein said biomarker is selected from the group consisting of: IGFBP3; IGFBP5, IGFBP6; AXL; and PDGFR-α; and
(b) comparing the level of said biomarker in said sample relative to a standard, wherein increased expression indicates a decreased likelihood said patient will respond therapeutically to a cancer treatment comprising the administration of an IGF-1R inhibitor, and recommending a more aggressive therapy.

8. The method according to claim 7, wherein said more aggressive therapy comprises a member of the group consisting of: (a) administering a higher dose of said cancer treatment; (b) administering said cancer treatment at an increased frequency; and (c) administering said cancer treatment in combination with another therapy.

9. A method of overcoming or preventing acquired resistance to an antibody IGF-1R inhibitor, comprising administering a combination of a second IGF-1R inhibitor with a PDGFR-α inhibitor.

10. The method according to claim 9, wherein said second IGF-1R inhibitor is BMS-754807.

11. A kit for use in treating a patient with cancer, comprising:
(a) a means for measuring whether a sample from said patient is positive for overexpression of one or more of: IGFBP3; IGFBP5; IGFBP6; AXL; and PDGFR-α;
(b) a therapeutically effective amount of an IGF-1R inhibitor in combination with a PDGFR-α inhibitor

12. The method according to claim 1, 2, 4 5, 7, 9, or 11, wherein said measurement is performed using a method selected from the group consisting of: (a) PCR; (b) RT-PCR; (c) FISH; (d) IHC; (e) immunodetection methods; (f) Western Blot; (g) ELISA; (h) radioimmuno assays; (i) immunoprecipitation; (j) FACS (k) HPLC; (1) surface plasmon resonance; (m) optical spectroscopy; and (i) mass spectrometry.

13. The method according to claim 1, 2, 4 5, 7, 9, or 11, wherein said cancer is a solid tumor, a metastatic tumor, breast cancer or lung cancer.

14. A method for treating a patient with cancer comprising administering a synergistic combination of a therapeutically acceptable amount of an IGF-1R inhibitor with a therapeutically acceptable amount of a PDGFR-α inhibitor.


SEQUENCE LISTING (36 sequences)

SEQ ID NO: 1 - DNA, 4104 bp, homo sapiens
atgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggagggaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctga

SEQ ID NO: 2 - protein, 1367 aa, homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 3 - DNA, 4104 bp, homo sapiens; identical to SEQ ID NO: 1

SEQ ID NO: 4 - protein, 1367 aa, homo sapiens; identical to SEQ ID NO: 2

SEQ ID NO: 5 - DNA, 3270 bp, homo sapiens
atggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccagatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgtcctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggatgacatcggcatagactcttcagacctggtggaagacagcttcctgtaa

SEQ ID NO: 6 - protein, 1089 aa, homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 7 - DNA, 3270 bp, homo sapiens; identical to SEQ ID NO: 5

SEQ ID NO: 8 - protein, 1089 aa, homo sapiens; identical to SEQ ID NO: 6

SEQ ID NO: 9 - DNA, 2658 bp, homo sapiens
atggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctga

SEQ ID NO: 10 - protein, 885 aa, homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 11 - DNA, 2685 bp, homo sapiens
atggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagggcaagcacagccagtccaccagctggtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctga

SEQ ID NO: 12 - protein, 894 aa, homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 13 - DNA, 2658 bp, homo sapiens; identical to SEQ ID NO: 9

SEQ ID NO: 14 - protein, 885 aa, homo sapiens; identical to SEQ ID NO: 10

SEQ ID NO: 15 - DNA, 2685 bp, homo sapiens; identical to SEQ ID NO: 11

SEQ ID NO: 16 - protein, 894 aa, homo sapiens; identical to SEQ ID NO: 12

SEQ ID NO: 17 - DNA, 876 bp, homo sapiens
atgcagcgggcgcgacccacgctctgggccgctgcgctgactctgctggtgctgctccgcgggccgccggtggcgcgggctggcgcgagctcggcgggcttgggtcccgtggtgcgctgcgagccgtgcgacgcgcgtgcactggcccagtgcgcgcctccgcccgccgtgtgcgcggagctggtgcgcgagccgggctgcggctgctgcctgacgtgcgcactgagcgagggccagccgtgcggcatctacaccgagcgctgtggctccggccttcgctgccagccgtcgcccgacgaggcgcgaccgctgcaggcgctgctggacggccgcgggctctgcgtcaacgctagtgccgtcagccgcctgcgcgcctacctgctgccagcgccgccagctccaggaaatgctagtgagtcggaggaagaccgcagcgccggcagtgtggagagcccgtccgtctccagcacgcaccgggtgtctgatcccaagttccaccccctccattcaaagataatcatcatcaagaaagggcatgctaaagacagccagcgctacaaagttgactacgagtctcagagcacagatacccagaacttctcctccgagtccaagcgggagacagaatatggtccctgccgtagagaaatggaagacacactgaatcacctgaagttcctcaatgtgctgagtcccaggggtgtacacattcccaactgtgacaagaagggattttataagaaaaagcagtgtcgcccttccaaaggcaggaagcggggcttctgctggtgtgtggataagtatgggcagcctctcccaggctacaccaccaaggggaaggaggacgtgcactgctacagcatgcagagcaagtag

SEQ ID NO: 18 - protein, 291 aa, homo sapiens
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 19 - DNA, 894 bp, homo sapiens
atgcagcgggcgcgacccacgctctgggccgctgcgctgactctgctggtgctgctccgcgggccgccggtggcgcgggctggcgcgagctcggcgggcttgggtcccgtggtgcgctgcgagccgtgcgacgcgcgtgcactggcccagtgcgcgcctccgcccgccgtgtgcgcggagctggtgcgcgagccgggctgcggctgctgcctgacgtgcgcactgagcgagggccagccgtgcggcatctacaccgagcgctgtggctccggccttcgctgccagccgtcgcccgacgaggcgcgaccgctgcaggcgctgctggacggccgcgggctctgcgtcaacgctagtgccgtcagccgcctgcgcgcctacctgctgccagcgccgccagctccaggtgagccgcccgcgccaggaaatgctagtgagtcggaggaagaccgcagcgccggcagtgtggagagcccgtccgtctccagcacgcaccgggtgtctgatcccaagttccaccccctccattcaaagataatcatcatcaagaaagggcatgctaaagacagccagcgctacaaagttgactacgagtctcagagcacagatacccagaacttctcctccgagtccaagcgggagacagaatatggtccctgccgtagagaaatggaagacacactgaatcacctgaagttcctcaatgtgctgagtcccaggggtgtacacattcccaactgtgacaagaagggattttataagaaaaagcagtgtcgcccttccaaaggcaggaagcggggcttctgctggtgtgtggataagtatgggcagcctctcccaggctacaccaccaaggggaaggaggacgtgcactgctacagcatgcagagcaagtag

SEQ ID NO: 20 - protein, 297 aa, homo sapiens
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGEPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 21 - DNA, 606 bp, homo sapiens
atggcgctgtcctgggttcttacagtcctgagcctcctacctctgctggaagcccagatcccattgtgtgccaacctagtaccggtgcccatcaccaacgccaccctggaccggatcactggcaagtggttttatatcgcatcggcctttcgaaacgaggagtacaataagtcggttcaggagatccaagcaaccttcttttacttcacccccaacaagacagaggacacgatctttctcagagagtaccagacccgacaggaccagtgcatctataacaccacctacctgaatgtccagcgggaaaatgggaccatctccagatacgtgggaggccaagagcatttcgctcacttgctgatcctcagggacaccaagacctacatgcttgcttttgacgtgaacgatgagaagaactgggggctgtctgtctatgctgacaagccagagacgaccaaggagcaactgggagagttctacgaagctctcgactgcttgcgcattcccaagtcagatgtcgtgtacaccgattggaaaaaggataagtgtgagccactggagaagcagcacgagaaggagaggaaacaggaggagggggaatcctag

SEQ ID NO: 22 - protein, 201 aa, homo sapiens
MALSWVLTVLSLLPLLEAQIPLCANLVPVPITNATLDRITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 23 - DNA, 2638 bp, homosapiens
agatgcgagcactgcggctgggcgctgaggatcagccgcttcctgcctggattccacagcttcgcgccgtgtactgtcgccccatccctgcgcgcccagcctgccaagcagcgtgccccggttgcaggcgtcatgcagcgggcgcgacccacgctctgggccgctgcgctgactctgctggtgctgctccgcgggccgccggtggcgcgggctggcgcgagctcggcgggcttgggtcccgtggtgcgctgcgagccgtgcgacgcgcgtgcactggcccagtgcgcgcctccgcccgccgtgtgcgcggagctggtgcgcgagccgggctgcggctgctgcctgacgtgcgcactgagcgagggccagccgtgcggcatctacaccgagcgctgtggctccggccttcgctgccagccgtcgcccgacgaggcgcgaccgctgcaggcgctgctggacggccgcgggctctgcgtcaacgctagtgccgtcagccgcctgcgcgcctacctgctgccagcgccgccagctccaggtgagccgcccgcgccaggaaatgctagtgagtcggaggaagaccgcagcgccggcagtgtggagagcccgtccgtctccagcacgcaccgggtgtctgatcccaagttccaccccctccattcaaagataatcatcatcaagaaagggcatgctaaagacagccagcgctacaaagttgactacgagtctcagagcacagatacccagaacttctcctccgagtccaagcgggagacagaatatggtccctgccgtagagaaatggaagacacactgaatcacctgaagttcctcaatgtgctgagtcccaggggtgtacacattcccaactgtgacaagaagggattttataagaaaaagcagtgtcgcccttccaaaggcaggaagcggggcttctgctggtgtgtggataagtatgggcagcctctcccaggctacaccaccaaggggaaggaggacgtgcactgctacagcatgcagagcaagtagacgcctgccgcaaggttaatgtggagctcaaatatgccttattttgcacaaaagactgccaaggacatgaccagcagctggctacagcctcgatttatatttctgtttgtggtgaactgattttttttaaaccaaagtttagaaagaggtttttgaaatgcctatggtttctttgaatggtaaacttgagcatcttttcactttccagtagtcagcaaagagcagtttgaattttcttgtcgcttcctatcaaaatattcagagactcgagcacagcacccagacttcatgcgcccgtggaatgctcaccacatgttggtcgaagcggccgaccactgactttgtgacttaggcggctgtgttgcctatgtagagaacacgcttcacccccactccccgtacagtgcgcacaggctttatcgagaataggaaaacctttaaaccccggtcatccggacatcccaacgcatgctcctggagctcacagccttctgtggtgtcatttctgaaacaagggcgtggatccctcaaccaagaagaatgtttatgtcttcaagtgacctgtactgcttggggactattggagaaaataaggtggagtcctacttgtttaaaaaatatgtatctaagaatgttctagggcactctgggaacctataaaggcaggtatttcgggccctcctcttcaggaatcttcctgaagacatggcccagtcgaaggcccaggatggcttttgctgcggccccgtggggtaggagggacagagagacagggagagtcagcctccacattcagaggcatcacaagtaatggcacaattcttcggatgactgcagaaaatagtgttttgtagttcaacaactcaagacgaagcttatttctgaggataagctctttaaaggcaaagctttattttcatctctcatcttttgtcctccttagcacaatgtaaaaaagaatagtaatatcagaacaggaaggaggaatggcttgctggggagcccatccaggacactgggagcacatagagattcacccatgtttgttgaacttagagtcattctcatgcttttctttataattcacacatatatgcagagaagatatgttcttgttaacattgtatacaacatagccccaaatatagtaagatctatactagataatcctagatgaaatgttagagatgctatatgatacaactgtggccatgactgaggaaaggagctcacgcccagagactgggctgctctcccggaggccaaacccaagaaggtctggcaaagtcaggctcagggagactctgccctgctgcagacctcggtgtggacacacgctgcatagagctctccttgaaaacagaggggtctcaagacattctgcctacctattagcttttctttatttttttaactttttggggggaaaagtatttttgagaagtttgtcttgcaatgtatttataaatagtaaataaagtttttaccattaaaaaaatatctttccctttgttattgaccatctctgggctttgtatcactaattattttattttattatataataattattttattataataaaatcctgaaaggggaaaataaaaaaaa

SEQ ID NO: 24 - protein, 297 aa, Homo sapiens; identical to SEQ ID NO: 20

SEQ ID NO: 25 - DNA, 777 bp, homosapiens
atgctgcccctctgcctcgtggccgccctgctgctggccgccgggcccgggccgagcctgggcgacgaagccatccactgcccgccctgctccgaggagaagctggcgcgctgccgcccccccgtgggctgcgaggagctggtgcgagagccgggctgcggctgttgcgccacttgcgccctgggcttggggatgccctgcggggtgtacaccccccgttgcggctcgggcctgcgctgctacccgccccgaggggtggagaagcccctgcacacactgatgcacgggcaaggcgtgtgcatggagctggcggagatcgaggccatccaggaaagcctgcagccctctgacaaggacgagggtgaccaccccaacaacagcttcagcccctgtagcgcccatgaccgcaggtgcctgcagaagcacttcgccaaaattcgagaccggagcaccagtgggggcaagatgaaggtcaatggggcgccccgggaggatgcccggcctgtgccccagggctcctgccagagcgagctgcaccgggcgctggagcggctggccgcttcacagagccgcacccacgaggacctctacatcatccccatccccaactgcgaccgcaacggcaacttccaccccaagcagtgtcacccagctctggatgggcagcgtggcaagtgctggtgtgtggaccggaagacgggggtgaagcttccggggggcctggagccaaagggggagctggactgccaccagctggctgacagctttcgagagtga

SEQ ID NO: 26 - protein, 258 aa, homosapiens
MLPLCLVAALLLAAGPGPSLGDEAIHCPPCSEEKLARCRPPVGCEELVREPGCGCCATCALGLGMPCGVYTPRCGSGLRCYPPRGVEKPLHTLMHGQGVCMELAEIEAIQESLQPSDKDEGDHPNNSFSPCSAHDRRCLQKHFAKIRDRSTSGGKMKVNGAPREDARPVPQGSCQSELHRALERLAASQSRTHEDLYIIPIPNCDRNGNFHPKQCHPALDGQRGKCWCVDRKTGVKLPGGLEPKGELDCHQLADSFRE

SEQ ID NO: 27 - DNA, 777 bp, homosapiens; identical to SEQ ID NO: 25

SEQ ID NO: 28 - protein, 258 aa, homosapiens; identical to SEQ ID NO: 26

SEQ ID NO: 29 - DNA, 819 bp, homosapiens
atggtgttgctcaccgcggtcctcctgctgctggccgcctatgcggggccggcccagagcctgggctccttcgtgcactgcgagccctgcgacgagaaagccctctccatgtgcccccccagccccctgggctgcgagctggtcaaggagccgggctgcggctgctgcatgacctgcgccctggccgaggggcagtcgtgcggcgtctacaccgagcgctgcgcccaggggctgcgctgcctcccccggcaggacgaggagaagccgctgcacgccctgctgcacggccgcggggtttgcctcaacgaaaagagctaccgcgagcaagtcaagatcgagagagactcccgtgagcacgaggagcccaccacctctgagatggccgaggagacctactcccccaagatcttccggcccaaacacacccgcatctccgagctgaaggctgaagcagtgaagaaggaccgcagaaagaagctgacccagtccaagtttgtcgggggagccgagaacactgcccacccccggatcatctctgcacctgagatgagacaggagtctgagcagggcccctgccgcagacacatggaggcttccctgcaggagctcaaagccagcccacgcatggtgccccgtgctgtgtacctgcccaattgtgaccgcaaaggattctacaagagaaagcagtgcaaaccttcccgtggccgcaagcgtggcatctgctggtgcgtggacaagtacgggatgaagctgccaggcatggagtacgttgacggggactttcagtgccacaccttcgacagcagcaacgttgagtga

SEQ ID NO: 30 - protein, 272 aa, homo sapiens
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMAEETYSPKIFRPKHTRISELKAEAVKKDRRKKLTQSKFVGGAENTAHPRIISAPEMRQESEQGPCRRHMEASLQELKASPRMVPRAVYLPNCDRKGFYKRKQCKPSRGRKRGICWCVDKYGMKLPGMEYVDGDFQCHTFDSSNVE

SEQ ID NO: 31 - DNA, 819 bp, homo sapiens; identical to SEQ ID NO: 29

SEQ ID NO: 32 - protein, 272 aa, homo sapiens; identical to SEQ ID NO: 30

SEQ ID NO: 33 - DNA, 723 bp, homo sapiens
atgaccccccacaggctgctgccaccgctgctgctgctgctagctctgctgctcgctgccagcccaggaggcgccttggcgcggtgcccaggctgcgggcaaggggtgcaggcgggttgtccagggggctgcgtggaggaggaggatggggggtcgccagccgagggctgcgcggaagctgagggctgtctcaggagggaggggcaggagtgcggggtctacacccctaactgcgccccaggactgcagtgccatccgcccaaggacgacgaggcgcctttgcgggcgctgctgctcggccgaggccgctgccttccggcccgcgcgcctgctgttgcagaggagaatcctaaggagagtaaaccccaagcaggcactgcccgcccacaggatgtgaaccgcagagaccaacagaggaatccaggcacctctaccacgccctcccagcccaattctgcgggtgtccaagacactgagatgggcccatgccgtagacatctggactcagtgctgcagcaactccagactgaggtctaccgaggggctcaaacactctacgtgcccaattgtgaccatcgaggcttctaccggaagcggcagtgccgctcctcccaggggcagcgccgaggtccctgctggtgtgtggatcggatgggcaagtccctgccagggtctccagatggcaatggaagctcctcctgccccactgggagtagcggctaa

SEQ ID NO: 34 - protein, 240 aa, homo sapiens
MTPHRLLPPLLLLLALLLAASPGGALARCPGCGQGVQAGCPGGCVEEEDGGSPAEGCAEAEGCLRREGQECGVYTPNCAPGLQCHPPKDDEAPLRALLLGRGRCLPARAPAVAEENPKESKPQAGTARPQDVNRRDQQRNPGTSTTPSQPNSAGVQDTEMGPCRRHLDSVLQQLQTEVYRGAQTLYVPNCDHRGFYRKRQCRSSQGQRRGPCWCVDRMGKSLPGSPDGNGSSSCPTGSSG

SEQ ID NO: 35 - DNA, 723 bp, homo sapiens; identical to SEQ ID NO: 33

SEQ ID NO: 36 - protein, 240 aa, homo sapiens; identical to SEQ ID NO: 34